(19) United States
(12) Patent Application Publication
(10) Pub. No.: US 2025/0243407 A1
(43) Pub. Date: Jul. 31, 2025

(54) LIQUID CRYSTAL COMPOSITION, OPTICALLY ANISOTROPIC FILM, OPTICAL FILM, POLARIZING PLATE, IMAGE DISPLAY APPARATUS, AND POLYMERIZABLE COMPOUND

(71) Applicant: FUJIFILM Corporation, Tokyo (JP)

(72) Inventors: Yuki HIRAI, Kanagawa (JP); Kouitsu SASAKI, Kanagawa (JP); Ryoji HIMENO, Kanagawa (JP); Shinichi MORISHIMA, Kanagawa (JP); Mayumi NOJIRI, Kanagawa (JP); Ayako MURAMATSU, Kanagawa (JP); Megumi OKUBO, Kanagawa (JP); Aiko YOSHIDA, Kanagawa (JP)

(73) Assignee: FUJIFILM Corporation, Tokyo (JP)

(21) Appl. No.: 19/076,590

(22) Filed: Mar. 11, 2025

Related U.S. Application Data
(63) Continuation of application No. PCT/JP2023/031369, filed on Aug. 30, 2023.

(30) Foreign Application Priority Data
Sep. 26, 2022 (JP) 2022-152376

(51) Int. Cl. C09K 19/38; C07D 339/06; C08F 222/24; C09K 19/04; G02B 5/30

(52) U.S. Cl. CPC C09K 19/3861; C07D 339/06; C08F 222/245; G02B 5/3016; C09K 2019/0448

(57) ABSTRACT

A liquid crystal composition used for forming an optically anisotropic film having excellent amine resistance, and an optically anisotropic film, an optical film, a polarizing plate, an image display apparatus, and a polymerizable compound. The liquid crystal composition contains a first polymerizable compound represented by Formula (1) and a second polymerizable compound represented by Formula (2), in which a content of the second polymerizable compound is 1% by mass or more with respect to a total mass of the first polymerizable compound and the second polymerizable compound.

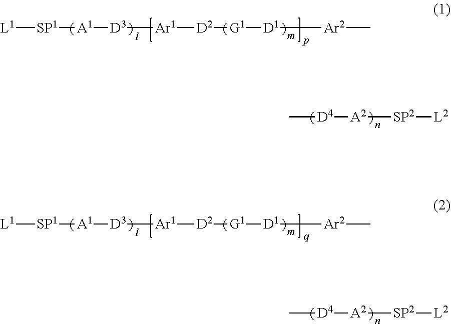

### CROSS-REFERENCE TO RELATED APPLICATIONS

This application is a Continuation of PCT International Application No. PCT/JP2023/031369 filed on Aug. 30, 2023, which was published under PCT Article 21(2) in Japanese, and which claims priority under 35 U.S.C. § 119(a) to Japanese Patent Application No. 2022-152376 filed on Sep. 26, 2022. The above applications are hereby expressly incorporated by reference, in their entirety, into the present application.

### BACKGROUND OF THE INVENTION

### 1. Field of the Invention

The present invention relates to a liquid crystal composition, an optically anisotropic film, an optical film, a polarizing plate, an image display apparatus, and a polymerizable compound.

### 2. Description of the Related Art

A polymerizable compound exhibiting reverse wavelength dispersibility can accurately convert ray wavelengths over a wide wavelength range and reduce in thickness of a retardation film due to its high refractive index, and the polymerizable compound has been actively studied.

In addition, for the polymerizable compound exhibiting reverse wavelength dispersibility, T-type molecular design guidelines have generally been adapted, and it has been required to decrease a wavelength of a major axis of a molecule and increase a wavelength of a minor axis positioned at a center of the molecule.

Therefore, it has been known that a cycloalkylene skeleton having no absorption wavelength is used for the connection between a minor axis skeleton positioned at the center of the molecule (hereinafter, also referred to as “reverse-wavelength dispersion expressing portion”) and the minor axis of the molecule.

For example, JP6823011B discloses a polymerizable composition which contains a polymerizable compound having two or more reverse-wavelength dispersion expressing portions and a polymerizable compound having two or more polymerizable groups ([Claim 1], [Claim 9], and the like]).

### SUMMARY OF THE INVENTION

As a result of studying the polymerizable composition described in JP6823011B, the present inventors have found that, depending on a structure of the polymerizable compounds to be blended, an optically anisotropic film to be formed has a problem of durability in which a birefringence index changes due to ammonia, which is a basic nucleophilic substance (hereinafter, also referred to as “amine resistance”).

Therefore, an object of the present invention is to provide a liquid crystal composition used for forming an optically anisotropic film having excellent amine resistance, and an optically anisotropic film, an optical film, a polarizing plate, an image display apparatus, and a polymerizable compound.

As a result of intensive studies to achieve the above object, the present inventors have found that, by using a liquid crystal composition in which a specific amount of a second polymerizable compound having three or more reverse-wavelength dispersion expressing portions in a molecule is blended together with a first polymerizable compound having two reverse-wavelength dispersion expressing portions, the amine resistance of the optically anisotropic film to be formed is improved, and have completed the present invention.

That is, the present inventors have found that the above-described object can be achieved by adopting the following configurations.

[1] A liquid crystal composition comprising:
a first polymerizable compound represented by Formula (1) described later; and
a second polymerizable compound represented by Formula (2) described later,
in which a content of the second polymerizable compound is 1% by mass or more with respect to a total mass of the first polymerizable compound and the second polymerizable compound.

[2] The liquid crystal composition according to [1],
in which Ar¹ and Ar² in Formulae (1) and (2) described later represent any aromatic ring selected from the group consisting of the groups represented by Formulae (Ar-1) to (Ar-4).

[3] The liquid crystal composition according to [1] or [2],
in which G1 in Formulae (1) and (2) described later represents A^{G}.

The liquid crystal composition according to any one of [1] to [3],
in which any one of Z¹ or Z² in Formulae (Ar-1) to (Ar-8) described later represents a tert-butyl group.

[5] The liquid crystal composition according to any one of [1] to [4],
in which G¹ in Formulae (1) and (2) described later represents a cycloalkane ring or a cycloalkene ring.

[6] The liquid crystal composition according to any one of [1] to [5],
in which, in Formulae (Ar-1) to (Ar-8) described later, Z¹ represents a hydrogen atom and Z² represents a tert-butyl group.

[7] The liquid crystal composition according to any one of [1] to [6],
in which, in Formulae (1) and (2) described later, both of 1 and n represent 1, and both of A¹ and A² represent a benzene ring.

[8] The liquid crystal composition according to any one of [1] to [7],
in which the content of the second polymerizable compound is 5% to 50% by mass with respect to the total mass of the first polymerizable compound and the second polymerizable compound.

[9] The liquid crystal composition according to any one of [1] to [8], further comprising:
a polymerizable compound having one or more polymerizable groups, which is different from the first polymerizable compound and the second polymerizable compound.

[10] An optically anisotropic film obtained by fixing an alignment state of the liquid crystal composition according to any one of [1] to [9].

[11] An optical film comprising:
the optically anisotropic film according to [10].

[12] A polarizing plate comprising:
the optical film according to [11]; and
a polarizer.

[13] An image display apparatus comprising:
the optical film according to [11].

[14] An image display apparatus comprising:
the polarizing plate according to [12].

[15] A polymerizable compound represented by Formula (2) described later.

According to the present invention, it is possible to provide a liquid crystal composition used for forming an optically anisotropic film having excellent amine resistance, and an optically anisotropic film, an optical film, a polarizing plate, an image display apparatus, and a polymerizable compound.

### BRIEF DESCRIPTION OF THE DRAWINGS

FIG. 1 is a schematic cross-sectional view showing an example of an optical film according to an embodiment of the present invention.

FIG. 2 is a schematic cross-sectional view showing another example of the optical film according to the embodiment of the present invention.

FIG. 3 is a schematic cross-sectional view showing still another example of the optical film according to the embodiment of the present invention.

### DESCRIPTION OF THE PREFERRED EMBODIMENTS

Hereinafter, the present invention will be described in detail.

The description of configuration requirements described below may be made based on typical embodiments of the present invention, but the present invention is not limited to such embodiments.

Any numerical range expressed using “to” in the present specification refers to a range including the numerical values before and after the “to” as a lower limit value and an upper limit value, respectively.

In addition, in the present specification, substances corresponding to respective components may be used alone or in combination of two or more kinds thereof. Here, in a case where two or more kinds of substances are used in combination for each component, the content of the component indicates the total content of the substances used in combination, unless otherwise specified.

In addition, in the present specification, “(meth)acrylate” denotes “acrylate” or “methacrylate”, “(meth)acryl” denotes “acryl” or “methacryl”, and “(meth)acryloyl” denotes “acryloyl” or “methacryloyl”.

In addition, in the present specification, a bonding direction of a divalent group (for example, —O—CO—) described is not particularly limited, and for example, in a case where L² in an “L¹—L²—L³” bond is —O—CO—, and a bonding position on the L¹ side is represented by *1 and a bonding position on the L³ side is represented by *2, L² may be *1-O—CO—*2 or *1-CO—O—*λ.

In addition, in the present specification, Re(λ) and Rth(λ) respectively represent an in-plane retardation at a wavelength λ and a thickness-direction retardation at a wavelength λ. Unless otherwise specified, the wavelength λ refers to 550 nm.

In the present invention, Re(λ) and Rth(λ) are values measured at the wavelength of λ in AxoScan (manufactured by Axometrics, Inc.). By inputting an average refractive index ((nx+ny+nz)/3) and a film thickness (d (μm)) in AxoScan,

Slow axis direction) (°),

Re (λ)=R0(λ), and

Rth(λ)=((nx+ny)/2−nz)×d

are calculated.

Although R0(λ) is displayed as a numerical value calculated by AxoScan, it means Re (λ).

In addition, in the present specification, examples of a substituent include substituents described in the following substituent group A.

In the present specification, “may have a substituent” includes not only an aspect of not having a substituent but also an aspect of having one or more substituents.

Substituent group A

Examples of the substituent include a halogen atom (for example, a fluorine atom, a chlorine atom, or a bromine atom; preferably a chlorine atom or a fluorine atom and more preferably a fluorine atom);

an alkyl group (a linear, branched, or cyclic alkyl group having preferably 1 to 48 carbon atoms, more preferably 1 to 24 carbon atoms, and particularly preferably 1 to 8 carbon atoms, such as a linear alkyl group having 1 to 6 carbon atoms (for example, a methyl group, an ethyl group, an n-propyl group, an n-butyl group, an n-pentyl group, and an n-hexyl group), a branched alkyl group having 3 to 6 carbon atoms (for example, an isopropyl group, an isobutyl group, a tert-butyl group, a sec-butyl group, a neopentyl group, an isohexyl group, and a 3-methylpentyl group), and a cyclic alkyl group having 3 to 12 carbon atoms (for example, a cyclopropyl group, a cyclopentyl group, a cyclohexyl group, a 1-norbornyl group, and a 1-adamantyl group));

an alkenyl group (an alkenyl group having preferably 2 to 48 carbon atoms and more preferably 2 to 18 carbon atoms, such as a vinyl group, an allyl group, a 1-butene group, and a 2-butene group);

an alkynyl group (an alkynyl group having preferably 2 to 6 carbon atoms and more preferably 2 to 4 carbon atoms, such as an ethynyl group, a 1-propynyl group, a propargyl group, a 1-butylnyl group, and a 2-butylnyl group);

an aryl group (an aryl group having preferably 6 to 48 carbon atoms and more preferably 6 to 24 carbon atoms, such as a phenyl group, an oligoaryl group (a naphthyl group or an anthryl group), a phenanthrenyl group, a fluorenyl group, a pyrenyl group, a triphenylenyl group, and a biphenyl group);

a heteroaryl group (a heterocyclic group having preferably 1 to 32 carbon atoms and more preferably 1 to 18 carbon atoms, such as a 2-thienyl group, a 4-pyridyl group, a 2-furyl group, a 2-pyrimidinyl group, a 1-pyridyl group, a 2-benzothiazolyl group, a 1-imidazolyl group, a 1-pyrazolyl group, and a benzotriazol-1-yl group);

an arylalkyl group (an arylalkyl group having preferably 7 to 15 carbon atoms, such as a benzyl group, a phenethyl group, a methylbenzyl group, a phenylpropyl group, a 1-methylphenylethyl group, a phenylbutyl group, a 2-methylphenylpropyl group, a tetrahydronaphthyl group, a naphthylmethyl group, a naphthylethyl group, an indenyl group, a fluorenyl group, an anthracenylmethyl group (an anthrylmethyl group), and a phenanthrenylmethyl group (a phenanthrylmethyl group));

a silyl group (a silyl group having preferably 3 to 38 carbon atoms and more preferably 3 to 18 carbon atoms, such as a trimethylsilyl group, a triethylsilyl group, a tributylsilyl group, a t-butyldimethylsilyl group, and a t-hexyldimethylsilyl group);

a hydroxy group; a cyano group; a nitro group; a morpholino group;

an alkoxy group (an alkoxy group having preferably 1 to 48 carbon atoms and more preferably 1 to 24 carbon atoms, such as a methoxy group, an ethoxy group, a 1-butoxy group, a 2-butoxy group, an isopropoxy group, a t-butoxy group, a dodecyloxy group, and a cycloalkyloxy group (for example, a cyclopentyloxy group or a cyclohexyloxy group));

an aryloxy group (an aryloxy group having preferably 6 to 48 carbon atoms and more preferably 6 to 24 carbon atoms, such as a phenoxy group and a 1-naphthoxy group);

an alkenyloxy group (an alkenyloxy group having preferably 2 to 6 carbon atoms, such as a vinyloxy group, a 1-propenyloxy group, a 2-n-propenyloxy group (an allyloxy group), a 1-n-butenyloxy group, and a prenyloxy group);

a heterocyclic oxy group (a heterocyclic oxy group having preferably 1 to 32 carbon atoms and more preferably 1 to 18 carbon atoms, such as a 1-phenyltetrazole-5-oxy group and a 2-tetrahydropyranyloxy group);

a silyloxy group (a silyloxy group having preferably 1 to 32 carbon atoms and more preferably 1 to 18 carbon atoms, such as a trimethylsilyloxy group, a t-butyldimethylsilyloxy group, and a diphenylmethylsilyloxy group);

an acyloxy group (an acyloxy group having preferably 2 to 48 carbon atoms and more preferably 2 to 24 carbon atoms, such as an acetoxy group, a pivaloyloxy group, a benzoyloxy group, a dodecanoyloxy group, an acryloyloxy group, and a methacryloyloxy group);

a hydroxyalkyleneoxy group (a hydroxyalkyleneoxy group having preferably 2 to 10 carbon atoms, such as a hydroxyethyleneoxy group);

an alkoxycarbonyloxy group (an alkoxycarbonyloxy group having preferably 2 to 48 carbon atoms and more preferably 2 to 24 carbon atoms, such as an ethoxycarbonyloxy group, a t-butoxycarbonyloxy group, and a cycloalkyloxycarbonyloxy group (for example, a cyclohexyloxycarbonyloxy group));

an aryloxycarbonyloxy group (an aryloxycarbonyloxy group having preferably 7 to 32 carbon atoms and more preferably 7 to 24 carbon atoms, such as a phenoxycarbonyloxy group);

a carbamoyloxy group (a carbamoyloxy group having preferably 1 to 48 carbon atoms and more preferably 1 to 24 carbon atoms, such as an N,N-dimethylcarbamoyloxy group, an N-butylcarbamoyloxy group, an N-phenylcarbamoyloxy group, and an N-ethyl-N-phenylcarbamoyloxy group);

a sulfamoyloxy group (a sulfamoyloxy group having preferably 1 to 32 carbon atoms and more preferably 1 to 24 carbon atoms, such as an N,N-diethylsulfamoyloxy group and an N-propylsulfamoyloxy group);

an alkylsulfonyloxy group (an alkylsulfonyloxy group having preferably 1 to 38 carbon atoms and more preferably 1 to 24 carbon atoms, such as a methylsulfonyloxy group, a hexadecylsulfonyloxy group, and a cyclohexylsulfonyloxy group);

an arylsulfonyloxy group (an arylsulfonyloxy group having preferably 6 to 32 carbon atoms and more preferably 6 to 24 carbon atoms, such as a phenylsulfonyloxy group);

an acyl group (an acyl group having preferably 1 to 48 carbon atoms and more preferably 1 to 24 carbon atoms, such as a formyl group, an acetyl group, an acryloyl group, a methacryloyl group, a pivaloyl group, a benzoyl group, a tetradecanoyl group, and a cyclohexanoyl group);

an alkoxycarbonyl group (an alkoxycarbonyl group having preferably 2 to 48 carbon atoms and more preferably 2 to 24 carbon atoms, such as a methoxycarbonyl group, an ethoxycarbonyl group, an octadecyloxycarbonyl group, a cyclohexyloxycarbonyl group, and a 2,6-di-tert-butyl-4-methylcyclohexyloxycarbonyl group);

an aryloxycarbonyl group (an aryloxycarbonyl group having preferably 7 to 32 carbon atoms and more preferably 7 to 24 carbon atoms, such as a phenoxycarbonyl group);

a carbamoyl group (a carbamoyl group having preferably 1 to 48 carbon atoms and more preferably 1 to 24 carbon atoms, such as a carbamoyl group, an N,N-diethylcarbamoyl group, an N-ethyl-N-octylcarbamoyl group, an N,N-dibutylcarbamoyl group, an N-propylcarbamoyl group, an N-phenylcarbamoyl group, an N-methyl-N-phenylcarbamoyl group, and an N,N-dicyclohexylcarbamoyl group);

an amino group (an amino group having preferably 32 or less carbon atoms and more preferably 24 or less carbon atoms, such as an amino group, a methylamino group, an N,N-dibutylamino group, a tetradecylamino group, a 2-ethylhexylamino group, and a cyclohexylamino group);

an anilino group (an anilino group having preferably 6 to 32 carbon atoms and more preferably 6 to 24 carbon atoms, such as an anilino group and an N-methylanilino group);

a heterocyclic amino group (a heterocyclic amino group having preferably 1 to 32carbon atoms and more preferably 1 to 18 carbon atoms, such as a 4-pyridylamino group);

a carbonamide group (a carbonamide group having preferably 2 to 48 carbon atoms and more preferably 2 to 24 carbon atoms, such as an acetamide group, a benzamide group, a tetradecaneamide group, a pivaloylamide group, and a cyclohexaneamide group);

a ureido group (a ureido group having preferably 1 to 32 carbon atoms and more preferably 1 to 24 carbon atoms, such as a ureido group, an N,N-dimethylureido group, and an N-phenylureido group);

an imide group (an imide group having preferably 36 or less carbon atoms and more preferably 24 or less carbon atoms, such as an N-succinimide group and an N-phthalimide group);

an alkoxycarbonylamino group (an alkoxycarbonylamino group having preferably 2 to 48 carbon atoms and more preferably 2 to 24 carbon atoms, such as a methoxycarbonylamino group, an ethoxycarbonylamino group, a t-butoxycarbonylamino group, an octadecyloxycarbonylamino group, and a cyclohexyloxycarbonylamino group);

an aryloxycarbonylamino group (an aryloxycarbonylamino group having preferably 7 to 32 carbon atoms and more preferably 7 to 24 carbon atoms, such as a phenoxycarbonylamino group);

a sulfonamide group (a sulfonamide group having preferably 1 to 48 carbon atoms and more preferably 1 to 24 carbon atoms, such as a methanesulfonamide group, a butanesulfonamide group, a benzenesulfonamide group, a hexadecanesulfonamide group, and a cyclohexanesulfonamide group);

a sulfamoylamino group (a sulfamoylamino group having preferably 1 to 48 carbon atoms and more preferably 1 to 24 carbon atoms, such as an N,N-dipropylsulfamoylamino group and an N-ethyl-N-dodecylsulfamoylamino group);

an azo group (an azo group having preferably 1 to 32 carbon atoms and more preferably 1 to 24 carbon atoms, such as a phenylazo group and a 3-pyrazolylazo group);

an alkylthio group (an alkylthio group having preferably 1 to 48 carbon atoms and more preferably 1 to 24 carbon atoms, such as a methylthio group, an ethylthio group, an octylthio group, and a cyclohexylthio group);

an arylthio group (an arylthio group having preferably 6 to 48 carbon atoms and more preferably 6 to 24 carbon atoms, such as a phenylthio group);

a heterocyclic thio group (a heterocyclic thio group having preferably 1 to 32 carbon atoms and more preferably 1 to 18 carbon atoms, such as a 2-benzothiazolylthio group, a 2-pyridylthio group, and a 1-phenyltetrazolylthio group);

an alkylsulfinyl group (an alkylsulfinyl group having preferably 1 to 32 carbon atoms and more preferably 1 to 24 carbon atoms, such as a dodecanesulfinyl group);

an arylsulfinyl group (an arylsulfinyl group having preferably 6 to 32 carbon atoms and more preferably 6 to 24 carbon atoms, such as a phenylsulfinyl group);

an alkylsulfonyl group (an alkylsulfonyl group having preferably 1 to 48 carbon atoms and more preferably 1 to 24 carbon atoms, such as a methylsulfonyl group, an ethylsulfonyl group, a propylsulfonyl group, a butylsulfonyl group, an isopropylsulfonyl group, a 2-ethylhexylsulfonyl group, a hexadecylsulfonyl group, an octylsulfonyl group, and a cyclohexylsulfonyl group);

an arylsulfonyl group (an arylsulfonyl group having preferably 6 to 48 carbon atoms and more preferably 6 to 24 carbon atoms, such as a phenylsulfonyl group and a 1-naphthylsulfonyl group);

a sulfamoyl group (a sulfamoyl group having preferably 32 or less carbon atoms and more preferably 24 or less carbon atoms, such as a sulfamoyl group, an N,N-dipropylsulfamoyl group, an N-ethyl-N-dodecylsulfamoyl group, an N-ethyl-N-phenylsulfamoyl group, an N-cyclohexylsulfamoyl group, and an N-(2-ethylhexyl) sulfamoyl group);

a phosphonyl group (a phosphonyl group having preferably 1 to 32 carbon atoms and more preferably 1 to 24 carbon atoms, such as a phenoxyphosphonyl group, an octyloxyphosphonyl group, and a phenylphosphonyl group);

a phosphinoylamino group (a phosphinoylamino group having preferably 1 to 32 carbon atoms and more preferably 1 to 24 carbon atoms, such as a diethoxyphosphinoylamino group and a dioctyloxyphosphinoylamino group);

epoxy group; —NHOCOCH₃; —SO₂NH C₂H₄OCH₃; and —NHSO₂CH₃,

in which two or more thereof may be combined.

These substituents may be further substituted with these substituents. In addition, in a case where two or more substituents are present, these may be the same or different from each other. Furthermore, if possible, these substituents may be bonded to each other to form a ring.

### Liquid Crystal Composition

The liquid crystal composition according to the embodiment of the present invention is a liquid crystal composition containing a first polymerizable compound represented by Formula (1) described later and a second polymerizable compound represented by Formula (2) described later.

In addition, in the liquid crystal composition according to the embodiment of the present invention, a content of the second polymerizable compound is 1% by mass or more with respect to the total mass of the first polymerizable compound and the second polymerizable compound.

In the present invention, as described above, by using a liquid crystal composition in which a specific amount of the second polymerizable compound is blended together with the first polymerizable compound, the amine resistance of the optically anisotropic film to be formed is improved.

The reason for this is not clear, but the present inventors presume as follows.

That is, it is presumed that, in the liquid crystal composition, by containing the second polymerizable compound having three or more reverse-wavelength dispersion expressing portions in a molecule, a liquid crystal temperature range is widened, a film can be formed on the low temperature side sufficiently from a transparent point, and thus an alignment degree is high, and a polymerized film in a state of high density is obtained; and as a result, the amine resistance of the optically anisotropic film is improved.

Hereinafter, the first polymerizable compound and the second polymerizable compound contained in the liquid crystal composition according to the embodiment of the present invention will be described in detail.

### First Polymerizable Compound

The first polymerizable compound contained in the liquid crystal composition according to the embodiment of the present invention is a compound represented by Formula (1).

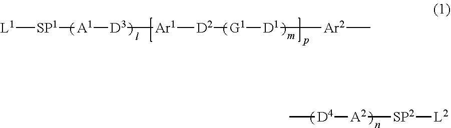

In Formula (1), D¹, D², D³, and D⁴ each independently represent a single bond, —CO—, —O—, —S—, —C(═S)—, —CR¹R²—, —CR³═CR⁴—, —NR⁵—, or a divalent linking group consisting of a combination of two or more of these groups, where R¹ to R⁵ each independently represent a hydrogen atom, a fluorine atom, or an alkyl group having 1 to 12 carbon atoms.

In addition, G¹ represents A^{G} or S^{PG}.

In addition, A¹, A², and A^{G} each independently represent an aromatic hydrocarbon ring which may have a substituent, an aromatic heterocyclic ring which may have a substituent, or a divalent alicyclic hydrocarbon group which may have a substituent. Here, one or more of —CH₂—'s constituting the alicyclic hydrocarbon group may be substituted with —O—, —S—, or —NH—.

In addition, SP¹, SP², and SP^{G} each independently represent a single bond or a divalent aliphatic hydrocarbon group having 1 to 20 carbon atoms. Here, one or more of —CH₂—'s constituting the aliphatic hydrocarbon group may be substituted with —O—, —S—, —NH—, —N(Q)—, or —CO—. Q represents a substituent.

In addition, L¹ and L² each independently represent a monovalent organic group, where at least one of L¹ or L² represents a polymerizable group. Here, in a case where at least one of Ar¹ or Ar² is an aromatic ring represented by Formula (Ar-4), at least one of L¹, L², or L³ or L⁴ in Formula (Ar-4) represents a polymerizable group.

In addition, m represents an integer of 0 to 2, where, in a case where m is 2, a plurality of G¹'s may be the same or different from each other, and a plurality of D¹'s may be the same or different from each other.

In addition, 1 and n each independently represent 0 or an integer of 1 or more, where, in a case where 1 is an integer of 2 or more, a plurality of A¹'s may be the same or different from each other, a plurality of D³'s may be the same or different from each other. In a case where n is an integer of 2 or more, a plurality of D⁴'s may be the same or different from each other, and a plurality of A²'s may be the same or different from each other.

In addition, p represents 1.

Here, an aspect in which all of G¹, D¹, and D² represent a single bond and an aspect in which D² represents a single bond and m represents 0 are excluded.

In Formula (1), examples of the divalent linking group represented by one aspect of D¹, D², D³, and D⁴ include —CO—, —O—, —CO—O—, —C(═S)O—, —CR¹R²—, —CR¹R²—CR¹R²—, —O—CR¹R²—, —CR¹R²—O—CR¹R2—, —CO—O—CR¹R²—, —O—CO—CR¹R²—, —CR¹R²—O—CO—CR¹R²—, —CR¹R²—CO—O—CR¹R²—, —NR⁵—CR¹R²—, and —CO—NR⁵—. R¹, R², and R⁵ each independently represent a hydrogen atom, a fluorine atom, or an alkyl group having 1 to 12 carbon atoms.

Among these, any of —CO—, —O—, or —CO—O— is preferable.

In Formula (1), examples of the aromatic hydrocarbon ring represented by one aspect of A¹, A², and A^{G} (A^{G} as one aspect of G¹; the same applies hereinafter) include an aromatic hydrocarbon ring having 6 to 20 carbon atoms; and specific examples thereof include a benzene ring, a naphthalene ring, an anthracene ring, and a phenanthroline ring.

In addition, examples of the aromatic heterocyclic ring represented by one aspect of A¹, A², and A^{G} include an aromatic heterocyclic ring having 5 to 20 carbon atoms; and specific examples thereof include a furan ring, a pyrrole ring, a thiophene ring, a pyridine ring, a thiazole ring, and a benzothiazole ring.

In addition, the divalent alicyclic hydrocarbon group represented by one aspect of A¹, A², and A^{G} is preferably a 5-membered ring or a 6-membered ring. In addition, the divalent alicyclic hydrocarbon group may be saturated or unsaturated, but a saturated divalent alicyclic hydrocarbon group is preferable. In addition, one or more of —CH₂—'s constituting the divalent alicyclic hydrocarbon group may be substituted with —O—, —S—, or —NH—. Examples of such a divalent alicyclic hydrocarbon group include a divalent alicyclic hydrocarbon group having 5 to 12 carbon atoms; and specific examples thereof include a monocyclic hydrocarbon group and a bridged cyclic hydrocarbon group, and more specific examples thereof include groups represented by Formulae (g-1) to (g-10).

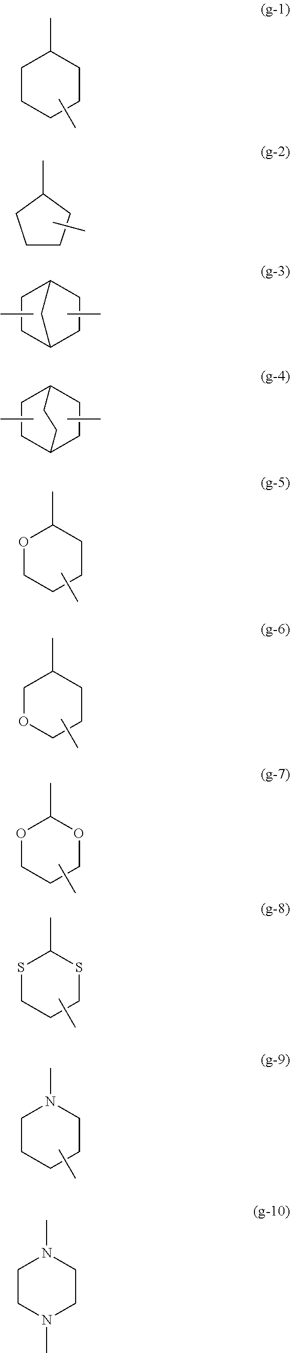

In addition, in Formula (1), examples of the substituent which may be included in the aromatic hydrocarbon ring, the aromatic heterocyclic ring, or the divalent alicyclic hydrocarbon group for A¹, A², and A^{G} include the substituents described in the substituent group A above; and among these, an alkyl group, an alkoxy group, an alkoxycarbonyl group, an alkylcarbonyloxy group, or a halogen atom is preferable.

In Formula (1), examples of the divalent aliphatic hydrocarbon group having 1 to 20 carbon atoms represented by one aspect of SP¹, SP², and SP^{G} (SP^{G} as one aspect G¹; the same applies hereinafter) include a linear or branched alkylene group having 1 to 20 carbon atoms, a linear or branched alkenylene group having 1 to 20 carbon atoms, and a linear or branched alkynylene group having 1 to 20 carbon atoms.

As the linear or branched alkylene group having 1 to 20 carbon atoms, an alkylene group having 1 to 12 carbon atoms is preferable and an alkylene group having 1 to 10 carbon atoms is more preferable; and suitable examples thereof include a methylene group, an ethylene group, a propylene group, a butylene group, a pentylene group, and a hexylene group.

As the linear or branched alkenylene group having 1 to 20 carbon atoms, an alkenylene group having 2 to 10 carbon atoms is preferable and an alkenylene group having 2 to 4 carbon atoms is more preferable; and suitable examples thereof include an ethenylene group.

As the linear or branched alkynylene group having 1 to 20 carbon atoms, an alkynylene group having 2 to 10 carbon atoms is preferable and an alkynylene group having 2 to 4 carbon atoms is more preferable; and suitable examples thereof include an ethynylene group.

As described above, in SP¹, SP², and SP^{G}, one or more of —CH₂—'s constituting the aliphatic hydrocarbon group may be substituted with —O—, —S—, —NH—, —N(Q)—, or —CO—; and examples of the substituent represented by Q include the substituents described in the substituent group A above. Among these, an alkyl group, an alkoxy group, an alkoxycarbonyl group, an alkylcarbonyloxy group, or a halogen atom is preferable.

In the present invention, from the viewpoint of easily exhibiting liquid crystallinity, it is preferable that G¹ in Formula (1) is A^{G} among A^{G} or SP^{G} described above.

In addition, from the reason that reverse wavelength dispersibility is improved and solubility is also favorable, it is preferable that G¹ in Formula (1) represents a cycloalkane ring or a cycloalkene ring.

Here, specific examples of the cycloalkane ring include a cyclohexane ring, a cyclopeptane ring, a cyclooctane ring, a cyclododecane ring, and a cyclodocosane ring.

In addition, specific examples of the cycloalkene ring include a cyclobutene ring, a cyclopentene ring, a cyclohexene ring, a cycloheptene ring, a cyclooctene ring, a cyclopentadiene ring, and a cyclohexadiene ring.

In Formula (1), examples of the monovalent organic group represented by L¹ and L² include an alkyl group, an aryl group, a heteroaryl group, an alkoxy group, a cyano group, and a carboxy group. The alkyl group may be linear, branched, or cyclic, but is preferably linear. The number of carbon atoms in the alkyl group is preferably 1 to 30, more preferably 1 to 20, and still more preferably 1 to 10. In addition, the aryl group may be monocyclic or polycyclic, but is preferably monocyclic. The number of carbon atoms in the aryl group is preferably 6 to 25 and more preferably 6 to 10. In addition, the heteroaryl group may be monocyclic or polycyclic. The number of heteroatoms constituting the heteroaryl group is preferably 1 to 3. The heteroatom constituting the heteroaryl group is preferably a nitrogen atom, a sulfur atom, or an oxygen atom. The number of carbon atoms in the heteroaryl group is preferably 6 to 18 and more preferably 6 to 12. In addition, the alkyl group, the aryl group, and the heteroaryl group may be unsubstituted or may have a substituent. Examples of the substituent include the substituents described in the substituent group A above; and among these, an alkyl group, an alkoxy group, an alkoxycarbonyl group, an alkylcarbonyloxy group, or a halogen atom is preferable.

In Formula (1), the polymerizable group represented by at least one of L¹ or L² is not particularly limited, but is preferably a polymerizable group capable of radical polymerization or cationic polymerization.

A known radically polymerizable group can be used as the radically polymerizable group, and suitable examples thereof include an acryloyloxy group or a methacryloyloxy group. In this case, it is known that the acryloyloxy group generally has a high polymerization rate, and from the viewpoint of improving productivity, the acryloyloxy group is preferable. However, the methacryloyloxy group can also be used as the polymerizable group.

A known cationically polymerizable group can be used as the cationically polymerizable group, and specific examples thereof include an alicyclic ether group, a cyclic acetal group, a cyclic lactone group, a cyclic thioether group, a spiro orthoester group, and a vinyloxy group. Among these, an alicyclic ether group or a vinyloxy group is suitable, and an epoxy group, an oxetanyl group, or a vinyloxy group is particularly preferable.

Examples of particularly preferred polymerizable group include a polymerizable group represented by any one of Formulae (P-1) to (P-20).

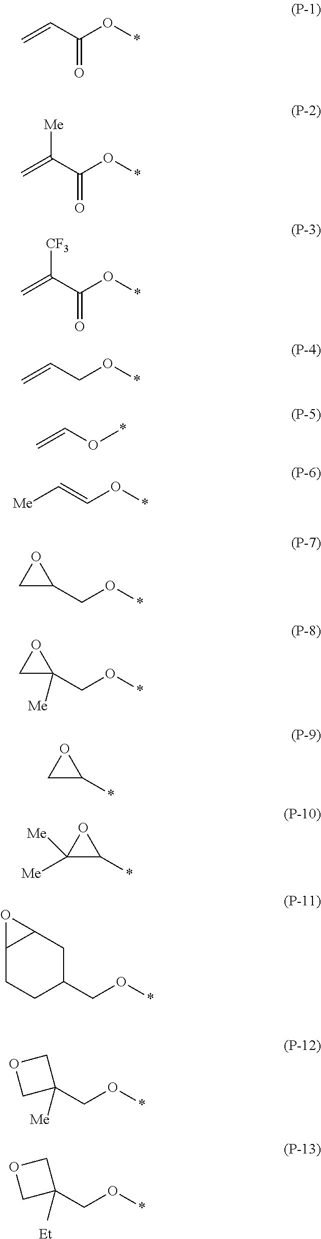

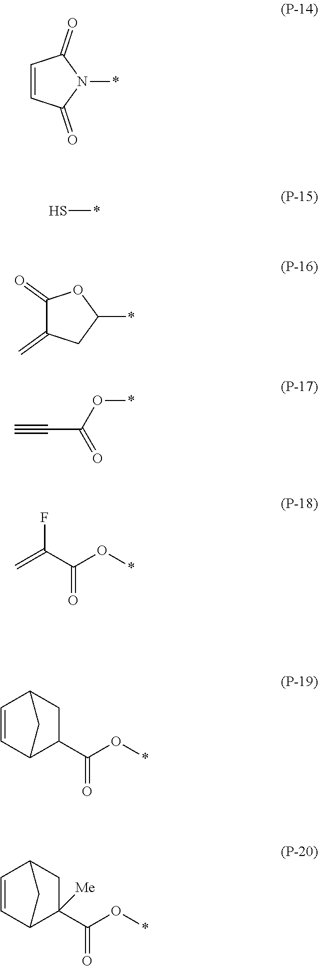

In Formula (1), from the reason that durability is favorable, it is preferable that both of L¹ and L² in Formula (I) are polymerizable groups, and it is more preferable that L¹ and L² are an acryloyl group or a methacryloyl group.

In Formula (1), m represents an integer of 0 to 2, 1 and n each independently represent an integer of 0 or 1 or more, and p represents 1.

Here, m is preferably 0 or 1, and from the viewpoint of synthesis, more preferably 1.

From the viewpoint of solubility and compatibility with other liquid crystal compounds, 1 and n are preferably an integer of 0 to 2.

In the present invention, from the viewpoint of easily exhibiting liquid crystallinity in a wide temperature range including room temperature, and increasing birefringence (Δn), it is preferable that both of 1 and n in Formula (1) represent 1 and both of A¹ and A² represent a benzene ring.

In the present invention, as described above, an aspect in which all of G¹, D¹, and D² in Formula (1) and Formula (2) described later represent a single bond, and an aspect in which D² in Formula (1) and Formula (2) described later represents a single bond and m represents 0 are excluded. That is, in the present invention, an aspect in which Ar¹ and Ar² are linked by a single bond is excluded.

On the other hand, in Formula (1), Ar¹ and Ar² each independently represent any aromatic ring selected from the group consisting of groups represented by Formulae (Ar-1) to (Ar-8). In Formulae (Ar-1) to (Ar-8), *1 represents a bonding position to D³ or D⁴, and *2 represents a bonding position to D¹ or D². Here, in a case where 1 is 0, the bonding position to D³ represents a bonding position to SP¹, in a case where m is 0, the bonding position to D¹ represents a bonding position to D², and in a case where n is 0, the bonding position to D⁴ represents a bonding position to SP².

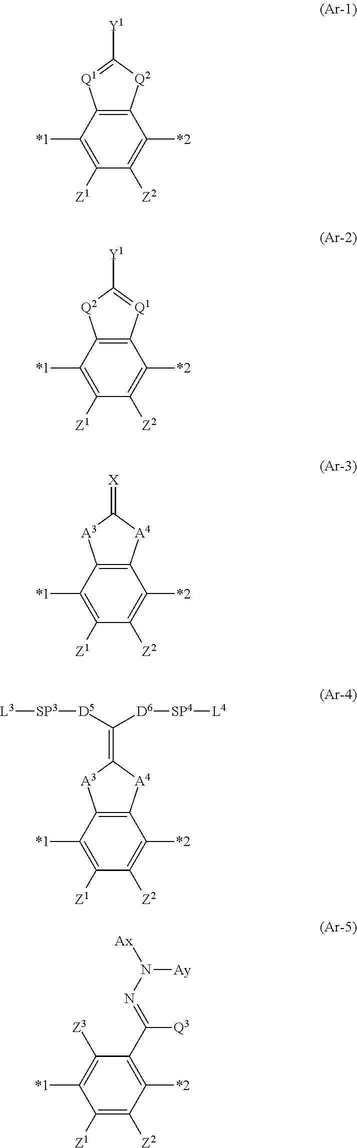

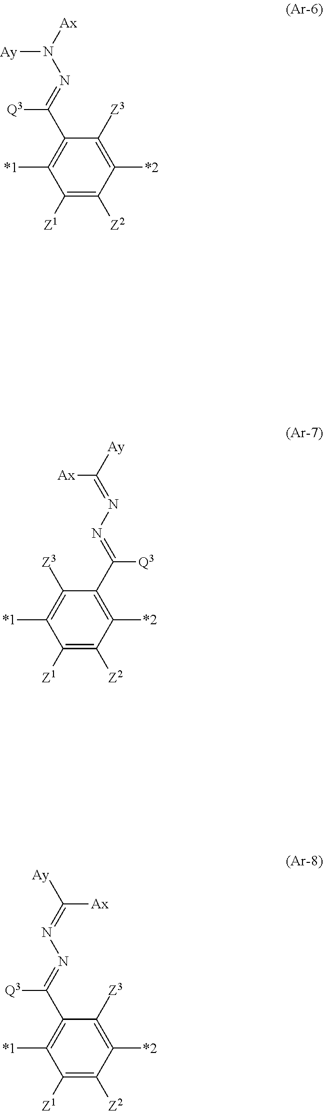

In Formulae (Ar-1) and (Ar-2), Q¹ represents N or CH, Q² represents —S—, —O—, or —N(R⁶)—, where R⁶ represents a hydrogen atom or an alkyl group having 1 to 6 carbon atoms, Y¹ represents an aromatic hydrocarbon group having 6 to 12 carbon atoms, which may have a substituent, an aromatic heterocyclic group having 3 to 12 carbon atoms, which may have a substituent, or an alicyclic hydrocarbon group having 6 to 20 carbon atoms, which may have a substituent, and one or more of —CH₂—'s constituting the alicyclic hydrocarbon group may be substituted with —O—, —S—, or —NH—.

Here, specific examples of the alkyl group having 1 to 6 carbon atoms represented by one aspect of R⁶ include a methyl group, an ethyl group, a propyl group, an isopropyl group, an n-butyl group, an isobutyl group, a sec-butyl group, a tert-butyl group, an n-pentyl group, and an n-hexyl group.

Examples of the aromatic hydrocarbon group having 6 to 12 carbon atoms, represented by one aspect of Y¹, include aryl groups such as a phenyl group, a 2,6-diethylphenyl group, and a naphthyl group.

Examples of the aromatic heterocyclic group having 3 to 12 carbon atoms, represented by one aspect of Y¹, include a heteroaryl group such as a thienyl group, a thiazolyl group, a furyl group, and a pyridyl group; and a group obtained by removing one hydrogen atom from any of an indole ring, a benzofuran ring, a benzothiophene ring, a benzimidazole ring, a benzothiazole ring, or a benzoxazole ring. Among these, as the aromatic heterocyclic group having 3 to 12 carbon atoms, represented by Y¹, a group obtained by removing one hydrogen atom from a benzofuran ring or a benzothiazole ring is preferable.

Examples of the alicyclic hydrocarbon group having 6 to 20 carbon atoms, represented by one aspect of Y¹, include a cyclohexylene group, a cyclopentylene group, a norbornylene group, and an adamantylene group.

In addition, examples of the substituent which may be included in Y¹ include the substituents described in the substituent group A above; and among these, an alkyl group, an alkoxy group, an alkoxycarbonyl group, an alkylcarbonyloxy group, or a halogen atom is preferable.

In addition, in Formulae (Ar-1) to (Ar-8), Z¹, Z², and Z³ each independently represent a hydrogen atom, a monovalent aliphatic hydrocarbon group having 1 to 20 carbon atoms, a monovalent alicyclic hydrocarbon group having 3 to 20 carbon atoms, a monovalent aromatic hydrocarbon group having 6 to 20 carbon atoms, a monovalent aromatic heterocyclic group having 6 to 20 carbon atoms, a halogen atom, a cyano group, a nitro group, —OR⁷, —NR⁸R⁹, —SR¹⁰, —COOR¹¹, or —COR¹², where R⁷ to R¹² each independently represent a hydrogen atom or an alkyl group having 1 to 6 carbon atoms, and Z¹ and Z² may be bonded to each other to form an aromatic ring.

As the monovalent aliphatic hydrocarbon group having 1 to 20 carbon atoms, an alkyl group having 1 to 15 carbon atoms is preferable, an alkyl group having 1 to 8 carbon atoms is more preferable; and specifically, a methyl group, an ethyl group, an isopropyl group, a tert-pentyl group (1,1-dimethylpropyl group), a tert-butyl group, or 1,1-dimethyl-3,3-dimethyl-butyl group is still more preferable and a methyl group, an ethyl group, or a tert-butyl group is particularly preferable.

Examples of the monovalent alicyclic hydrocarbon group having 3 to 20 carbon atoms include monocyclic saturated hydrocarbon groups such as a cyclopropyl group, a cyclobutyl group, a cyclopentyl group, a cyclohexyl group, a cycloheptyl group, a cyclooctyl group, a cyclodecyl group, a methylcyclohexyl group, and an ethylcyclohexyl group; monocyclic unsaturated hydrocarbon groups such as a cyclobutenyl group, a cyclopentenyl group, a cyclohexenyl group, a cycloheptenyl group, a cyclooctenyl group, a cyclodecenyl group, a cyclopentadienyl group, a cyclohexadienyl group, a cyclooctadienyl group, and a cyclodecadiene group; and polycyclic saturated hydrocarbon groups such as a bicyclo[2.2.1]heptyl group, a bicyclo[2.2.2]octyl group, a tricyclo[5.2.1.0^{2,6}]decyl group, a tricyclo[3.3.1.1^{3,7}]decyl group, a tetracyclo[6.2.1.1^{3,6}.0^{2,7}]dodecyl group, and an adamantyl group.

Specific examples of the monovalent aromatic hydrocarbon group having 6 to 20 carbon atoms include a phenyl group, a 2,6-diethylphenyl group, a naphthyl group, and a biphenyl group, and an aryl group having 6 to 12 carbon atoms (particularly, a phenyl group) is preferable.

Specific examples of the monovalent aromatic heterocyclic group having 6 to 20 carbon atoms include a 4-pyridyl group, a 2-furyl group, a 2-thienyl group, a 2-pyrimidinyl group, and a 2-benzothiazolyl group.

Examples of the halogen atom include a fluorine atom, a chlorine atom, a bromine atom, and an iodine atom, and among these, a fluorine atom, a chlorine atom, or a bromine atom is preferable.

On the other hand, specific examples of the alkyl group having 1 to 6 carbon atoms represented by one aspect of R⁷ to R¹⁰ include a methyl group, an ethyl group, a propyl group, an isopropyl group, an n-butyl group, an isobutyl group, a sec-butyl group, a tert-butyl group, an n-pentyl group, and an n-hexyl group.

As described above, Z¹ and Z² may be bonded to each other to form an aromatic ring, and examples of a structure in a case where Z¹ and Z² in Formula (Ar-1) are bonded to each other to form an aromatic ring include a group represented by Formula (Ar-1a). In Formula (Ar-1a), * represents a bonding position to D¹ or D² in Formula (I).

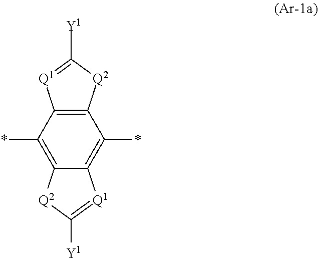

Here, in Formula (Ar-1a), examples of Q¹, Q², and Y¹ include the same as those described in Formula (Ar-1) above.

In the present invention, from the viewpoint of easily exhibiting liquid crystallinity, improving solubility, and enhancing durability (particularly, amine resistance) of the optically anisotropic film to be formed, it is preferable that any one of Z¹ or Z² in Formulae (Ar-1) to (Ar-8) represents a monovalent aliphatic hydrocarbon group having 1 to 20 carbon atoms (particularly, a tert-butyl group).

In addition, from the viewpoint of further improving the durability of the optically anisotropic film to be formed, it is preferable that, in Formulae (Ar-1) to (Ar-8), Z¹ represents a hydrogen atom and Z² represents a monovalent aliphatic hydrocarbon group having 1 to 20 carbon atoms (particularly, a tert-butyl group).

In addition, in Formulae (Ar-3) and (Ar-4), A³ and A⁴ each independently represent a group selected from the group consisting of —O—, —N(R¹³)—, —S—, and —CO—, and R¹³ represents a hydrogen atom or a substituent.

Examples of the substituent represented by one aspect of R¹³ include the substituents described in the substituent group A above; and among these, an alkyl group, an alkoxy group, an alkoxycarbonyl group, an alkylcarbonyloxy group, or a halogen atom is preferable.

In addition, in Formula (Ar-3), X represents a non-metal atom of Group 14 to Group 16. Here, a hydrogen atom or a substituent may be bonded to the non-metal atom.

In addition, examples of the non-metal atom of Groups 14 to 16, represented by X, include an oxygen atom, a sulfur atom, a nitrogen atom to which a hydrogen atom or a substituent is bonded [═N—R^{N1}, R^{N1} represents a hydrogen atom or a substituent], and a carbon atom to which a hydrogen atom or a substituent is bonded [═C—(R^{C1})₂, R^{C1} represents a hydrogen atom or a substituent].

Examples of the substituent include the substituents described in the substituent group A above; and among these, suitable examples thereof include an alkyl group, an alkoxy group, an alkyl-substituted alkoxy group, a cyclic alkyl group, an aryl group (for example, a phenyl group, a naphthyl group, or the like), a cyano group, an amino group, a nitro group, an alkylcarbonyl group, a sulfo group, and a hydroxyl group.

In addition, in Formula (Ar-4), D⁵ and D⁶ each independently represent a single bond, —CO—, —O—, —S—, —C(═S)—, —CR¹R²—, —CR³═CR⁴—, —NR⁵—, or a divalent linking group consisting of a combination of two or more of these groups, where R¹ to R⁵ each independently represent a hydrogen atom, a fluorine atom, or an alkyl group having 1 to 12 carbon atoms.

Here, examples of the divalent linking group include the same as those described for D¹, D², D³, and D⁴ in Formula (1) above.

In addition, in Formula (Ar-4), SP³ and SP⁴ each independently represent a single bond or a divalent aliphatic hydrocarbon group having 1 to 20 carbon atoms. Here, one or more of —CH₂—'s constituting the aliphatic hydrocarbon group may be substituted with —O—, —S—, —NH—, —N(Q)—, or —CO—. Q represents a substituent. Examples of the substituent represented by Q include the substituents described in the substituent group A above; and among these, an alkyl group, an alkoxy group, an alkoxycarbonyl group, an alkylcarbonyloxy group, or a halogen atom is preferable.

Here, examples of the divalent aliphatic hydrocarbon group include the same as those described for SP¹, SP², and SP^{G} in Formula (1) above.

L³ and L⁴ in Formula (Ar-4) each independently represent a monovalent organic group, and at least one of L³, L⁴, or L¹ or L² in Formula (1) above represents a polymerizable group.

Here, examples of the monovalent organic group include the same as those described for L¹ and L² in Formula (1) above.

In addition, examples of the polymerizable group include the same as those described for L¹ and L² in Formula (1) above.

In addition, in Formulae (Ar-5) to (Ar-8), Ax represents an organic group having 2 to 30 carbon atoms, which has at least one aromatic ring selected from the group consisting of an aromatic hydrocarbon ring and an aromatic heterocyclic ring.

In addition, in Formulae (Ar-5) to (Ar-8), Ay represents a hydrogen atom, an alkyl group having 1 to 12 carbon atoms, which may have a substituent, or an organic group having 2 to 30 carbon atoms which has at least one aromatic ring selected from the group consisting of an aromatic hydrocarbon ring and an aromatic heterocyclic ring.

Here, the aromatic rings in Ax and Ay may have a substituent, and Ax and Ay may be bonded to each other to form a ring.

In addition, Q³ represents a hydrogen atom or an alkyl group having 1 to 6 carbon atoms, which may have a substituent.

Examples of Ax and Ay include those described in paragraphs to of WO2014/010325A.

In addition, specific examples of the alkyl group having 1 to 6 carbon atoms, represented by Q³, include a methyl group, an ethyl group, a propyl group, an isopropyl group, an n-butyl group, an isobutyl group, a sec-butyl group, a tert-butyl group, an n-pentyl group, and an n-hexyl group; and examples of the substituent include the substituents described in the substituent group A above. Among these, an alkyl group, an alkoxy group, an alkoxycarbonyl group, an alkylcarbonyloxy group, or a halogen atom is preferable.

In the present invention, for the reason that durability (particularly, light resistance) of the optically anisotropic film to be formed is improved, it is preferable that Ar¹ and Ar² in Formula (1) above represent any aromatic ring selected from the group consisting of the groups represented by Formulae (Ar-1) to (Ar-4) above.

Examples of the first polymerizable compound represented by Formula (1) include the following compounds represented by Formulae (I) to (XII); and specific examples thereof include compounds having groups shown in Tables 1 to 8 as D¹, G¹, and D² and K in Formulae (I) to (VI), and compounds having groups shown in Table 9 as D¹, G¹, G¹, and D² and K in Formulae (VII) to (XII).

In Tables 1 to 8, “*” shown in a group such as D¹ represents a bonding position.

In addition, in the following description, a compound represented by Formula (I) and having a group shown as 1-1 in Table 1 is denoted by “Compound (I-1-1)”, and compounds having other structural formulae and groups are also denoted in the same manner. For example, a compound represented by Formula (II) and having a group shown as 2-3 in Table 2 can be denoted by “Compound (II-2-3)”.

In addition, in the compound (I-1-1) and the like, a group adjacent to an acryloyloxy group represents a propylene group (a group in which a methyl group is substituted with an ethylene group), and represents a mixture of regioisomers in which positions of the methyl groups are different.

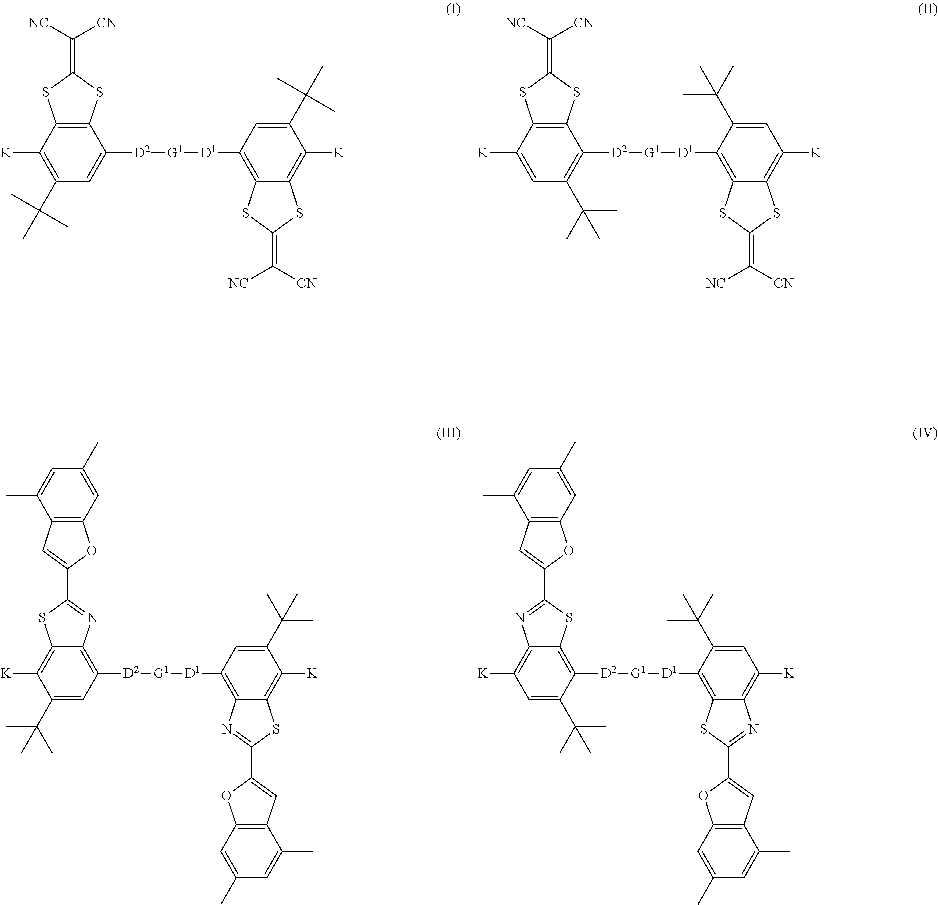

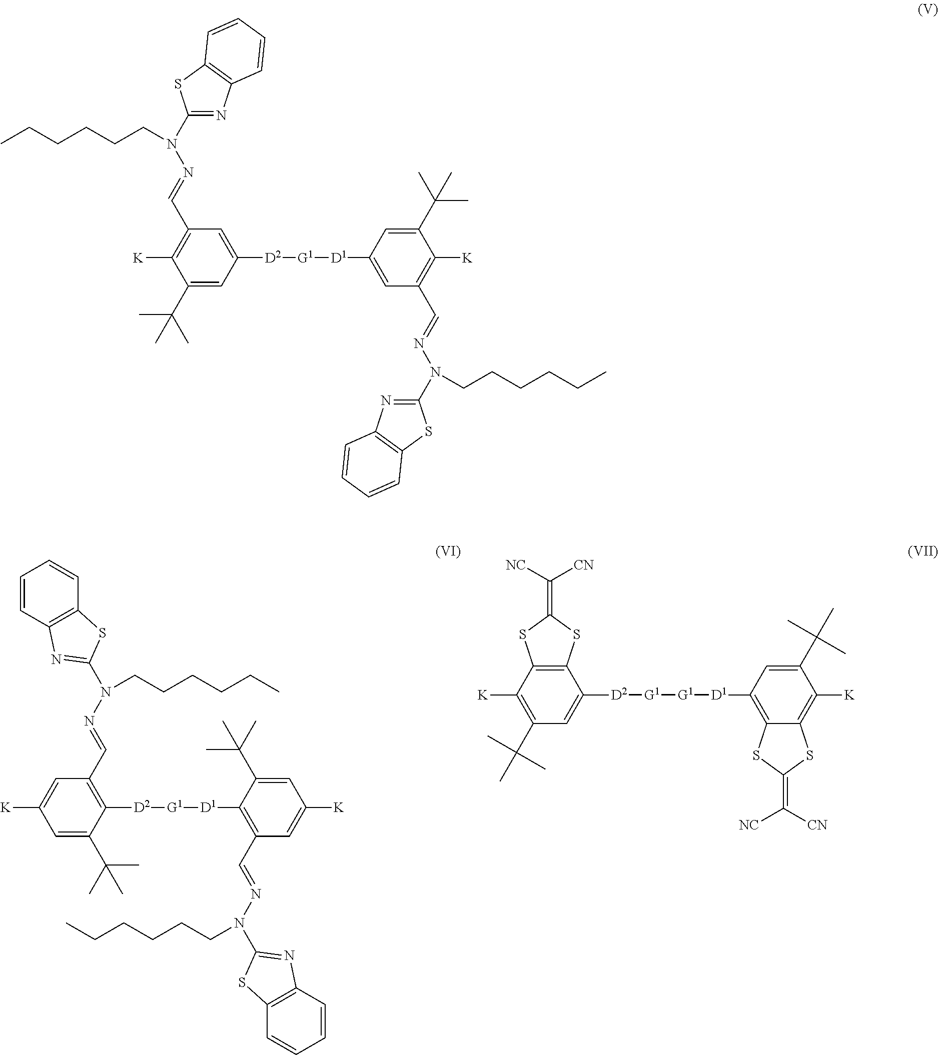

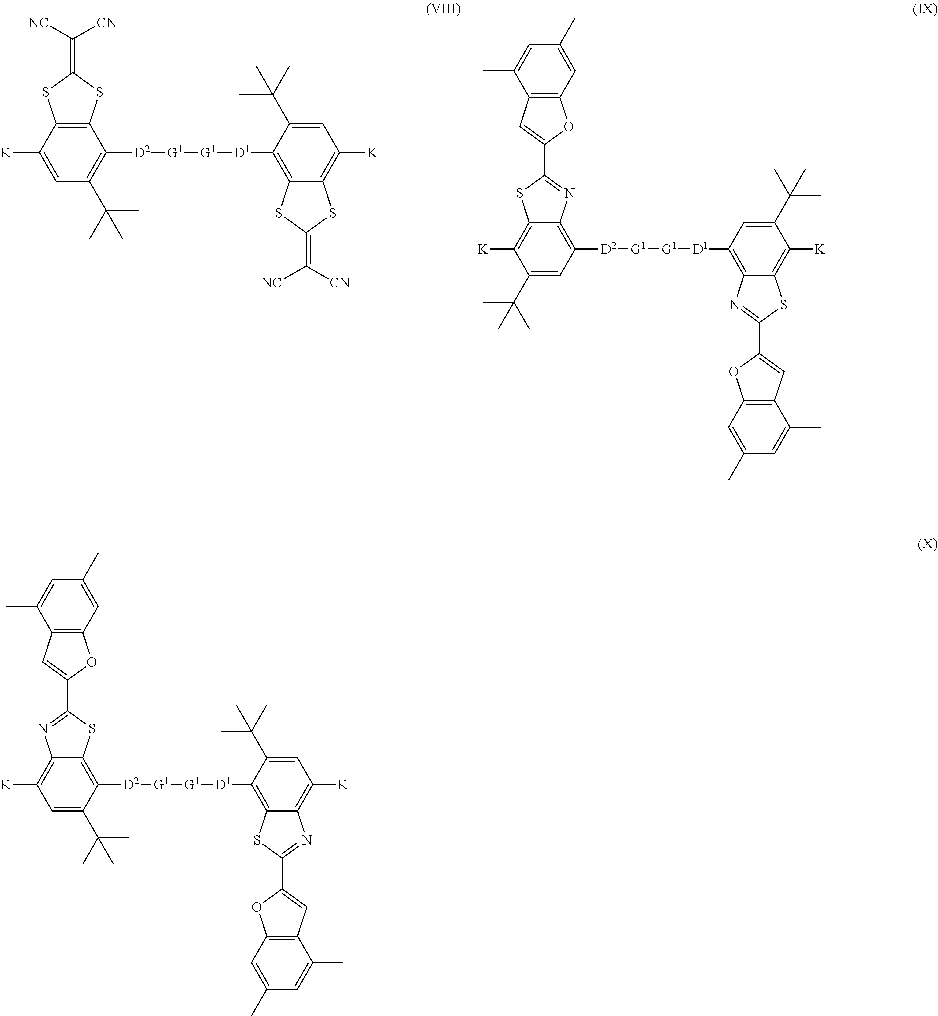

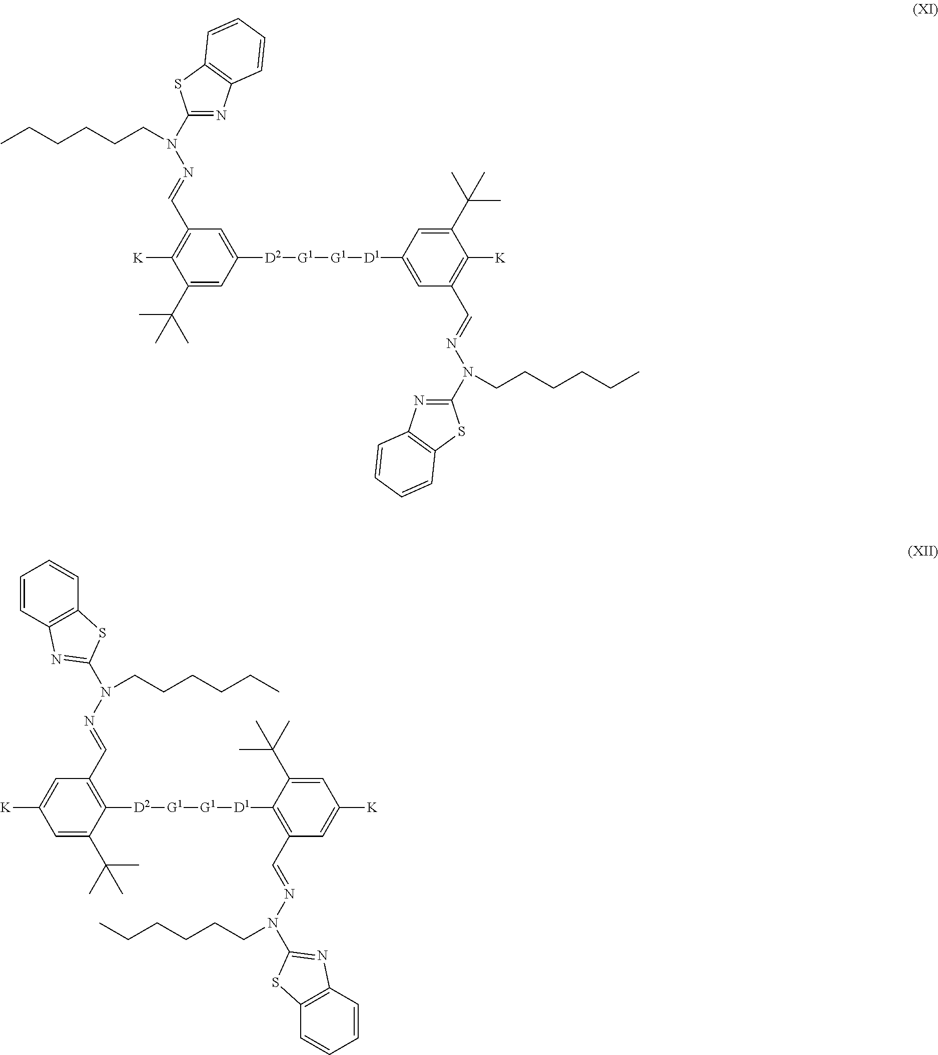

TABLE 1

| | D¹ | G¹ | D² | K |
|---|---|---|---|---|
| 1-1 | 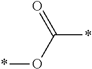 [SMILES: *OC(*)=O] | 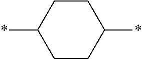 [SMILES: *C1CCC(*)CC1] | 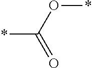 [SMILES: *OC(*)=O] | 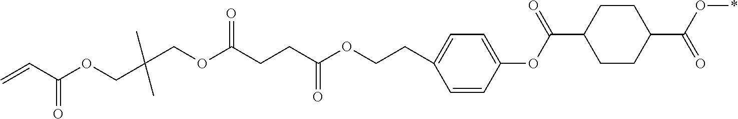 [SMILES: *OC(=O)C1CCC(C(=O)Oc2ccc(CCOC(=O)CCC(=O)OCC(C)(C)COC(=O)C=C)cc2)CC1] |
| 1-2 | 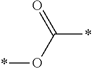 [SMILES: *OC(*)=O] | 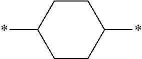 [SMILES: *C1CCC(*)CC1] | 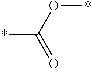 [SMILES: *OC(*)=O] | 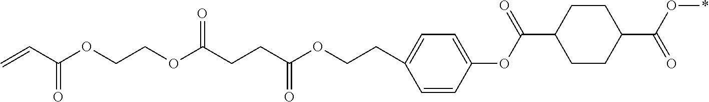 [SMILES: *OC(=O)C1CCC(C(=O)Oc2ccc(CCOC(=O)CCC(=O)OCCOC(=O)C=C)cc2)CC1] |
| 1-3 | 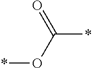 [SMILES: *OC(*)=O] | 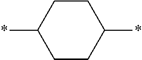 [SMILES: *C1CCC(*)CC1] | 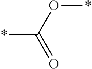 [SMILES: *OC(*)=O] | 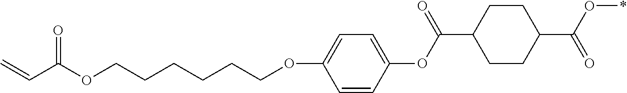 [SMILES: *OC(=O)C1CCC(C(=O)Oc2ccc(OCCCCCCOC(=O)C=C)cc2)CC1] |
| 1-4 | 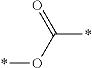 [SMILES: *OC(*)=O] | 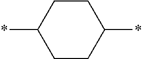 [SMILES: *C1CCC(*)CC1] | 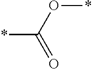 [SMILES: *OC(*)=O] | 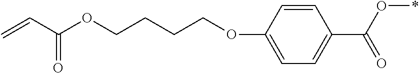 [SMILES: *OC(=O)c1ccc(OCCCCOC(=O)C=C)cc1] |
| 1-5 | 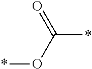 [SMILES: *OC(*)=O] | 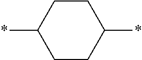 [SMILES: *C1CCC(*)CC1] | 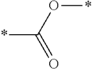 [SMILES: *OC(*)=O] | 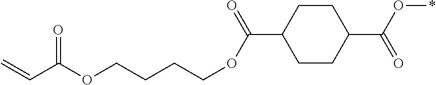 [SMILES: *OC(=O)C1CCC(C(=O)OCCCCOC(=O)C=C)CC1] |
| 1-6 | 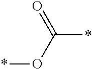 [SMILES: *OC(*)=O] | 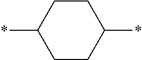 [SMILES: *C1CCC(*)CC1] | 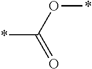 [SMILES: *OC(*)=O] | 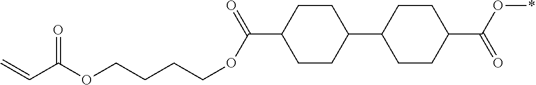 [SMILES: *OC(=O)C1CCC(C2CCC(C(=O)OCCCCOC(=O)C=C)CC2)CC1] |
| 1-7 | 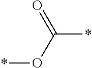 [SMILES: *OC(*)=O] | 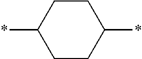 [SMILES: *C1CCC(*)CC1] | 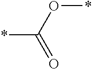 [SMILES: *OC(*)=O] | 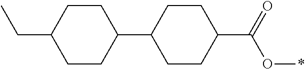 [SMILES: *OC(=O)C1CCC(C2CCC(CC)CC2)CC1] |
| 1-8 | 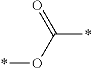 [SMILES: *OC(*)=O] | 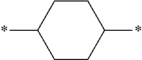 [SMILES: *C1CCC(*)CC1] | 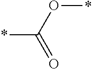 [SMILES: *OC(*)=O] | 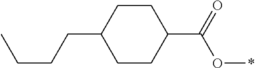 [SMILES: *OC(=O)C1CCC(CCCC)CC1] |
| 1-9 | 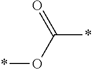 [SMILES: *OC(*)=O] | 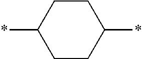 [SMILES: *C1CCC(*)CC1] | 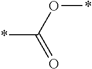 [SMILES: *OC(*)=O] | 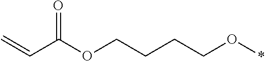 [SMILES: *OCCCCOC(=O)C=C] |
| 1-10 | 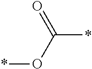 [SMILES: *OC(*)=O] | 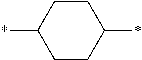 [SMILES: *C1CCC(*)CC1] | 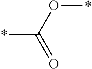 [SMILES: *OC(*)=O] | 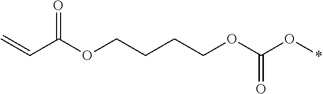 [SMILES: *OC(=O)OCCCCOC(=O)C=C] |

TABLE 2

| | D¹ | G¹ | D² | K |
|---|---|---|---|---|
| 2-1 | 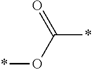 [SMILES: *OC(*)=O] | 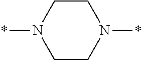 [SMILES: *N1CCN(*)CC1] | 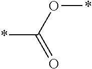 [SMILES: *OC(*)=O] | 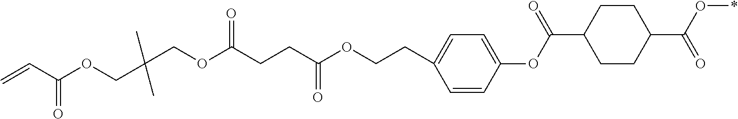 [SMILES: *OC(=O)C1CCC(C(=O)Oc2ccc(CCOC(=O)CCC(=O)OCC(C)(C)COC(=O)C=C)cc2)CC1] |
| 2-2 | 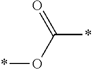 [SMILES: *OC(*)=O] | 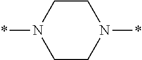 [SMILES: *N1CCN(*)CC1] | 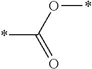 [SMILES: *OC(*)=O] | 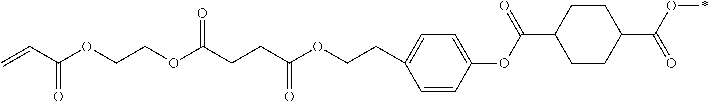 [SMILES: *OC(=O)C1CCC(C(=O)Oc2ccc(CCOC(=O)CCC(=O)OCCOC(=O)C=C)cc2)CC1] |
| 2-3 | 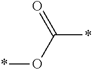 [SMILES: *OC(*)=O] | 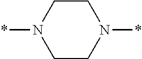 [SMILES: *N1CCN(*)CC1] | 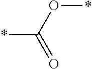 [SMILES: *OC(*)=O] | 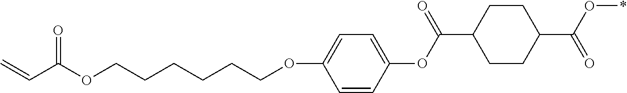 [SMILES: *OC(=O)C1CCC(C(=O)Oc2ccc(OCCCCCCOC(=O)C=C)cc2)CC1] |
| 2-4 | 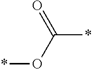 [SMILES: *OC(*)=O] | 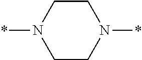 [SMILES: *N1CCN(*)CC1] | 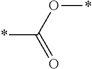 [SMILES: *OC(*)=O] | 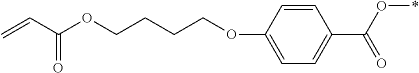 [SMILES: *OC(=O)c1ccc(OCCCCOC(=O)C=C)cc1] |
| 2-5 | 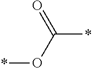 [SMILES: *OC(*)=O] | 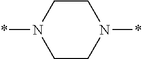 [SMILES: *N1CCN(*)CC1] | 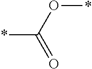 [SMILES: *OC(*)=O] | 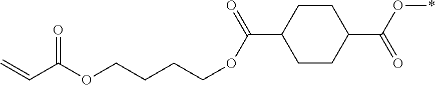 [SMILES: *OC(=O)C1CCC(C(=O)OCCCCOC(=O)C=C)CC1] |
| 2-6 | 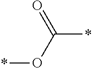 [SMILES: *OC(*)=O] | 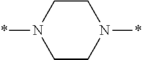 [SMILES: *N1CCN(*)CC1] | 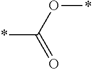 [SMILES: *OC(*)=O] | 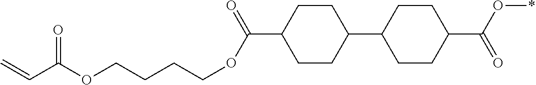 [SMILES: *OC(=O)C1CCC(C2CCC(C(=O)OCCCCOC(=O)C=C)CC2)CC1] |
| 2-7 | 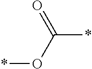 [SMILES: *OC(*)=O] | 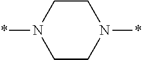 [SMILES: *N1CCN(*)CC1] | 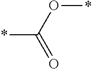 [SMILES: *OC(*)=O] | 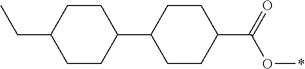 [SMILES: *OC(=O)C1CCC(C2CCC(CC)CC2)CC1] |
| 2-8 | 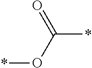 [SMILES: *OC(*)=O] | 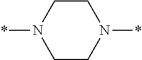 [SMILES: *N1CCN(*)CC1] | 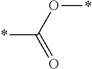 [SMILES: *OC(*)=O] | 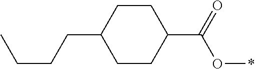 [SMILES: *OC(=O)C1CCC(CCCC)CC1] |
| 2-9 | 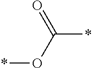 [SMILES: *OC(*)=O] | 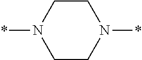 [SMILES: *N1CCN(*)CC1] | 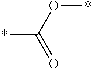 [SMILES: *OC(*)=O] | 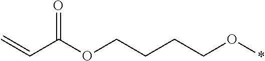 [SMILES: *OCCCCOC(=O)C=C] |
| 2-10 | 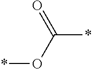 [SMILES: *OC(*)=O] | 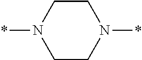 [SMILES: *N1CCN(*)CC1] | 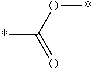 [SMILES: *OC(*)=O] | 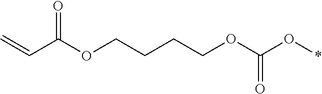 [SMILES: *OC(=O)OCCCCOC(=O)C=C] |

TABLE 3

| | D¹ | G¹ | D² | K |
|---|---|---|---|---|
| 3-1 | 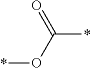 [SMILES: *OC(*)=O] | 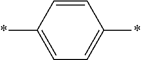 [SMILES: *c1ccc(*)cc1] | 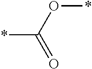 [SMILES: *OC(*)=O] | 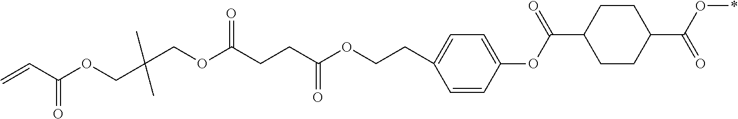 [SMILES: *OC(=O)C1CCC(C(=O)Oc2ccc(CCOC(=O)CCC(=O)OCC(C)(C)COC(=O)C=C)cc2)CC1] |
| 3-2 | 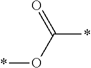 [SMILES: *OC(*)=O] | 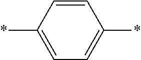 [SMILES: *c1ccc(*)cc1] | 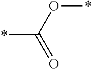 [SMILES: *OC(*)=O] | 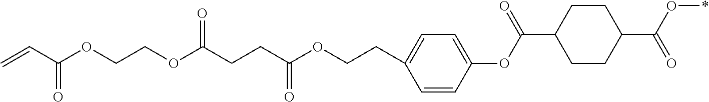 [SMILES: *OC(=O)C1CCC(C(=O)Oc2ccc(CCOC(=O)CCC(=O)OCCOC(=O)C=C)cc2)CC1] |
| 3-3 | 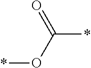 [SMILES: *OC(*)=O] | 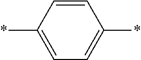 [SMILES: *c1ccc(*)cc1] | 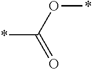 [SMILES: *OC(*)=O] | 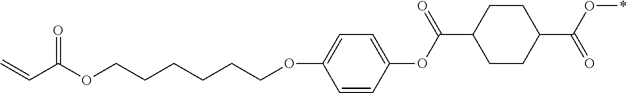 [SMILES: *OC(=O)C1CCC(C(=O)Oc2ccc(OCCCCCCOC(=O)C=C)cc2)CC1] |
| 3-4 | 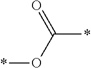 [SMILES: *OC(*)=O] | 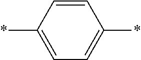 [SMILES: *c1ccc(*)cc1] | 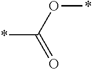 [SMILES: *OC(*)=O] | 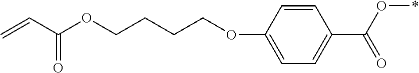 [SMILES: *OC(=O)c1ccc(OCCCCOC(=O)C=C)cc1] |
| 3-5 | 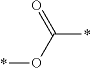 [SMILES: *OC(*)=O] | 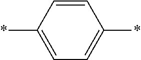 [SMILES: *c1ccc(*)cc1] | 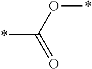 [SMILES: *OC(*)=O] | 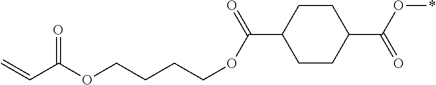 [SMILES: *OC(=O)C1CCC(C(=O)OCCCCOC(=O)C=C)CC1] |
| 3-6 | 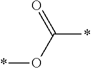 [SMILES: *OC(*)=O] | 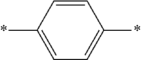 [SMILES: *c1ccc(*)cc1] | 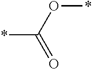 [SMILES: *OC(*)=O] | 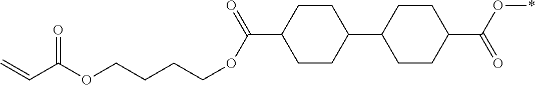 [SMILES: *OC(=O)C1CCC(C2CCC(C(=O)OCCCCOC(=O)C=C)CC2)CC1] |
| 3-7 | 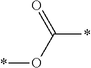 [SMILES: *OC(*)=O] | 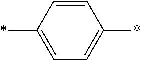 [SMILES: *c1ccc(*)cc1] | 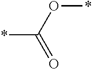 [SMILES: *OC(*)=O] | 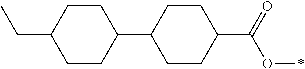 [SMILES: *OC(=O)C1CCC(C2CCC(CC)CC2)CC1] |
| 3-8 | 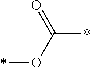 [SMILES: *OC(*)=O] | 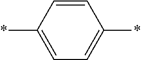 [SMILES: *c1ccc(*)cc1] | 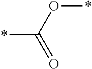 [SMILES: *OC(*)=O] | 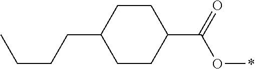 [SMILES: *OC(=O)C1CCC(CCCC)CC1] |
| 3-9 | 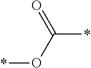 [SMILES: *OC(*)=O] | 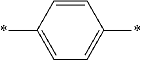 [SMILES: *c1ccc(*)cc1] | 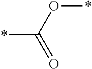 [SMILES: *OC(*)=O] | 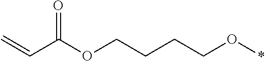 [SMILES: *OCCCCOC(=O)C=C] |
| 3-10 | 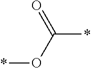 [SMILES: *OC(*)=O] | 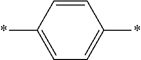 [SMILES: *c1ccc(*)cc1] | 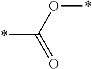 [SMILES: *OC(*)=O] | 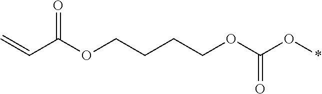 [SMILES: *OC(=O)OCCCCOC(=O)C=C] |

TABLE 4

| | D¹ | G¹ | D² | K |
|---|---|---|---|---|
| 4-1 | 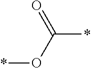 [SMILES: *OC(*)=O] | 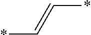 [SMILES: */C=C/*] | 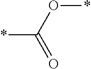 [SMILES: *OC(*)=O] | 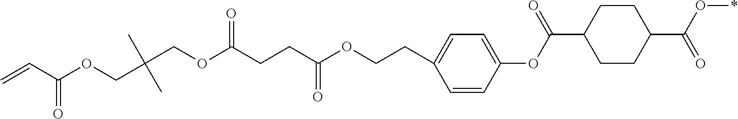 [SMILES: *OC(=O)C1CCC(C(=O)Oc2ccc(CCOC(=O)CCC(=O)OCC(C)(C)COC(=O)C=C)cc2)CC1] |
| 4-2 | 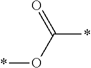 [SMILES: *OC(*)=O] | 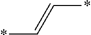 [SMILES: */C=C/*] | 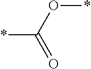 [SMILES: *OC(*)=O] | 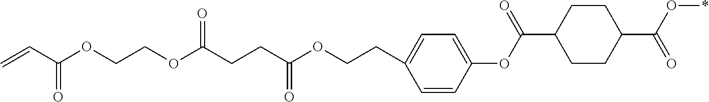 [SMILES: *OC(=O)C1CCC(C(=O)Oc2ccc(CCOC(=O)CCC(=O)OCCOC(=O)C=C)cc2)CC1] |
| 4-3 | 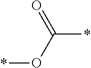 [SMILES: *OC(*)=O] | 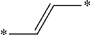 [SMILES: */C=C/*] | 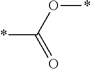 [SMILES: *OC(*)=O] | 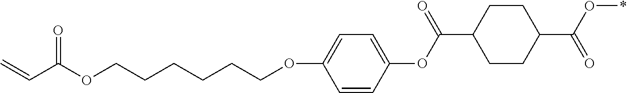 [SMILES: *OC(=O)C1CCC(C(=O)Oc2ccc(OCCCCCCOC(=O)C=C)cc2)CC1] |
| 4-4 | 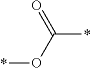 [SMILES: *OC(*)=O] | 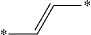 [SMILES: */C=C/*] | 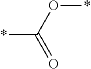 [SMILES: *OC(*)=O] | 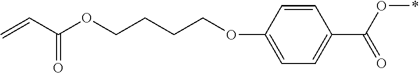 [SMILES: *OC(=O)c1ccc(OCCCCOC(=O)C=C)cc1] |
| 4-5 | 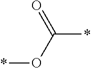 [SMILES: *OC(*)=O] | 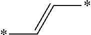 [SMILES: */C=C/*] | 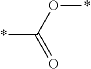 [SMILES: *OC(*)=O] | 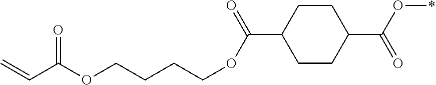 [SMILES: *OC(=O)C1CCC(C(=O)OCCCCOC(=O)C=C)CC1] |
| 4-6 | 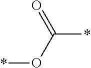 [SMILES: *OC(*)=O] | 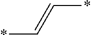 [SMILES: */C=C/*] | 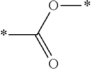 [SMILES: *OC(*)=O] | 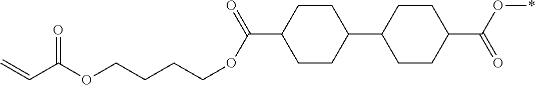 [SMILES: *OC(=O)C1CCC(C2CCC(C(=O)OCCCCOC(=O)C=C)CC2)CC1] |
| 4-7 | 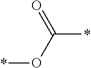 [SMILES: *OC(*)=O] | 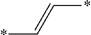 [SMILES: */C=C/*] | 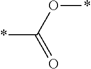 [SMILES: *OC(*)=O] | 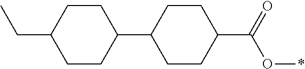 [SMILES: *OC(=O)C1CCC(C2CCC(CC)CC2)CC1] |
| 4-8 | 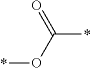 [SMILES: *OC(*)=O] | 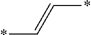 [SMILES: */C=C/*] | 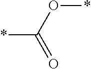 [SMILES: *OC(*)=O] | 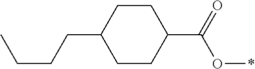 [SMILES: *OC(=O)C1CCC(CCCC)CC1] |
| 4-9 | 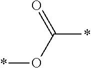 [SMILES: *OC(*)=O] | 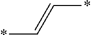 [SMILES: */C=C/*] | 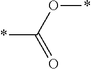 [SMILES: *OC(*)=O] | 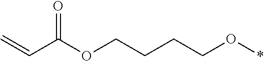 [SMILES: *OCCCCOC(=O)C=C] |
| 4-10 | 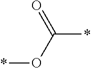 [SMILES: *OC(*)=O] | 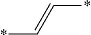 [SMILES: */C=C/*] | 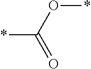 [SMILES: *OC(*)=O] | 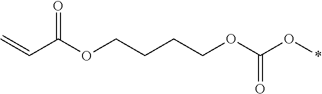 [SMILES: *OC(=O)OCCCCOC(=O)C=C] |

TABLE 5

| | D¹ | G¹ | D² | K |
|---|---|---|---|---|
| 5-1 | 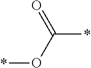 [SMILES: *OC(*)=O] | 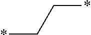 [SMILES: *CC*] | 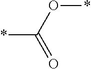 [SMILES: *OC(*)=O] | 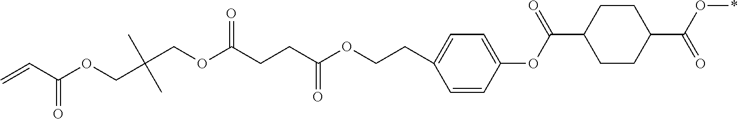 [SMILES: *OC(=O)C1CCC(C(=O)Oc2ccc(CCOC(=O)CCC(=O)OCC(C)(C)COC(=O)C=C)cc2)CC1] |
| 5-2 | 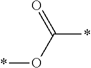 [SMILES: *OC(*)=O] | 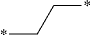 [SMILES: *CC*] | 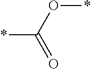 [SMILES: *OC(*)=O] | 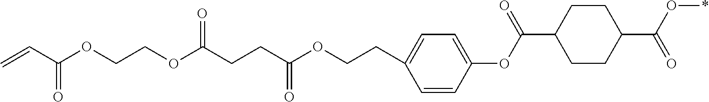 [SMILES: *OC(=O)C1CCC(C(=O)Oc2ccc(CCOC(=O)CCC(=O)OCCOC(=O)C=C)cc2)CC1] |
| 5-3 | 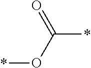 [SMILES: *OC(*)=O] | 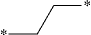 [SMILES: *CC*] | 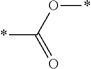 [SMILES: *OC(*)=O] | 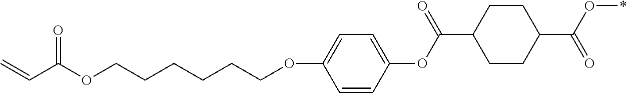 [SMILES: *OC(=O)C1CCC(C(=O)Oc2ccc(OCCCCCCOC(=O)C=C)cc2)CC1] |
| 5-4 | 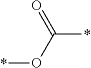 [SMILES: *OC(*)=O] | 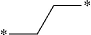 [SMILES: *CC*] | 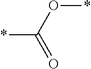 [SMILES: *OC(*)=O] | 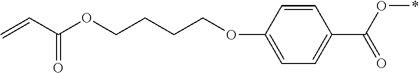 [SMILES: *OC(=O)c1ccc(OCCCCOC(=O)C=C)cc1] |
| 5-5 | 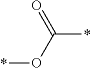 [SMILES: *OC(*)=O] | 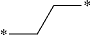 [SMILES: *CC*] | 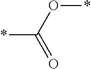 [SMILES: *OC(*)=O] | 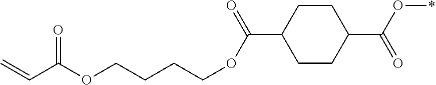 [SMILES: *OC(=O)C1CCC(C(=O)OCCCCOC(=O)C=C)CC1] |
| 5-6 | 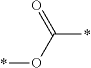 [SMILES: *OC(*)=O] | 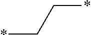 [SMILES: *CC*] | 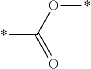 [SMILES: *OC(*)=O] | 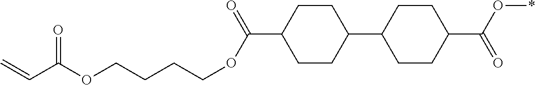 [SMILES: *OC(=O)C1CCC(C2CCC(C(=O)OCCCCOC(=O)C=C)CC2)CC1] |
| 5-7 | 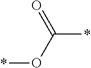 [SMILES: *OC(*)=O] | 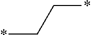 [SMILES: *CC*] | 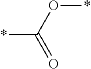 [SMILES: *OC(*)=O] | 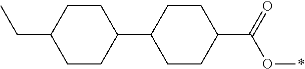 [SMILES: *OC(=O)C1CCC(C2CCC(CC)CC2)CC1] |
| 5-8 | 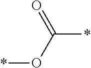 [SMILES: *OC(*)=O] | 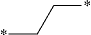 [SMILES: *CC*] | 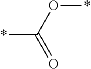 [SMILES: *OC(*)=O] | 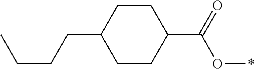 [SMILES: *OC(=O)C1CCC(CCCC)CC1] |
| 5-9 | 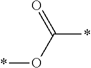 [SMILES: *OC(*)=O] | 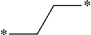 [SMILES: *CC*] | 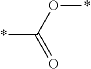 [SMILES: *OC(*)=O] | 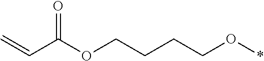 [SMILES: *OCCCCOC(=O)C=C] |
| 5-10 | 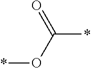 [SMILES: *OC(*)=O] | 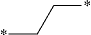 [SMILES: *CC*] | 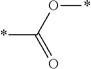 [SMILES: *OC(*)=O] | 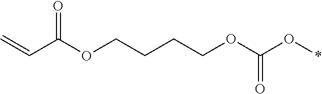 [SMILES: *OC(=O)OCCCCOC(=O)C=C] |

TABLE 6

| | D¹ | G¹ | D² | K |
|---|---|---|---|---|
| 6-1 | 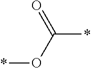 [SMILES: *OC(*)=O] | Single bond | 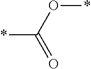 [SMILES: *OC(*)=O] | 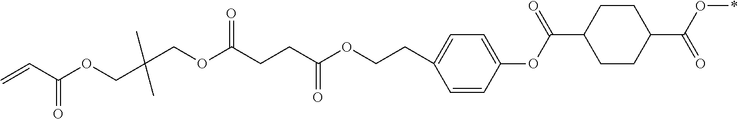 [SMILES: *OC(=O)C1CCC(C(=O)Oc2ccc(CCOC(=O)CCC(=O)OCC(C)(C)COC(=O)C=C)cc2)CC1] |
| 6-2 | 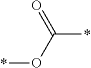 [SMILES: *OC(*)=O] | Single bond | 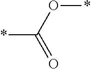 [SMILES: *OC(*)=O] | 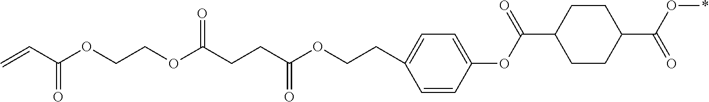 [SMILES: *OC(=O)C1CCC(C(=O)Oc2ccc(CCOC(=O)CCC(=O)OCCOC(=O)C=C)cc2)CC1] |
| 6-3 | 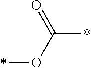 [SMILES: *OC(*)=O] | Single bond | 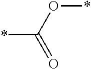 [SMILES: *OC(*)=O] | 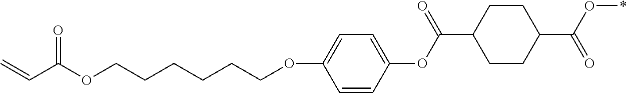 [SMILES: *OC(=O)C1CCC(C(=O)Oc2ccc(OCCCCCCOC(=O)C=C)cc2)CC1] |
| 6-4 | 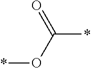 [SMILES: *OC(*)=O] | Single bond | 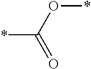 [SMILES: *OC(*)=O] | 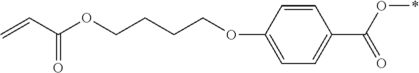 [SMILES: *OC(=O)c1ccc(OCCCCOC(=O)C=C)cc1] |
| 6-5 | 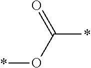 [SMILES: *OC(*)=O] | Single bond | 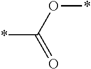 [SMILES: *OC(*)=O] | 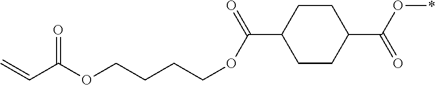 [SMILES: *OC(=O)C1CCC(C(=O)OCCCCOC(=O)C=C)CC1] |
| 6-6 | 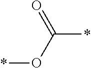 [SMILES: *OC(*)=O] | Single bond | 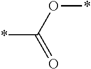 [SMILES: *OC(*)=O] | 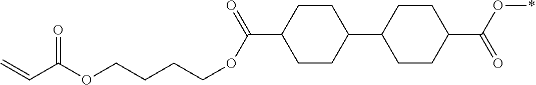 [SMILES: *OC(=O)C1CCC(C2CCC(C(=O)OCCCCOC(=O)C=C)CC2)CC1] |
| 6-7 | 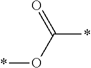 [SMILES: *OC(*)=O] | Single bond | 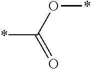 [SMILES: *OC(*)=O] | 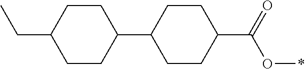 [SMILES: *OC(=O)C1CCC(C2CCC(CC)CC2)CC1] |
| 6-8 | 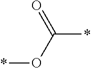 [SMILES: *OC(*)=O] | Single bond | 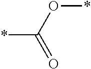 [SMILES: *OC(*)=O] | 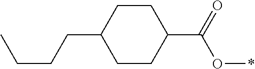 [SMILES: *OC(=O)C1CCC(CCCC)CC1] |
| 6-9 | 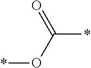 [SMILES: *OC(*)=O] | Single bond | 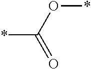 [SMILES: *OC(*)=O] | 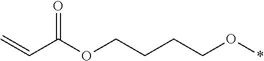 [SMILES: *OCCCCOC(=O)C=C] |
| 6-10 | 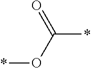 [SMILES: *OC(*)=O] | Single bond | 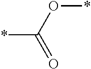 [SMILES: *OC(*)=O] | 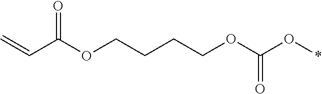 [SMILES: *OC(=O)OCCCCOC(=O)C=C] |

TABLE 7

| | D¹ | G¹ | D² | K |
|---|---|---|---|---|
| 7-1 | 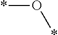 [SMILES: *O*] | 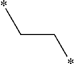 [SMILES: *CC*] | 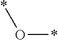 [SMILES: *O*] | 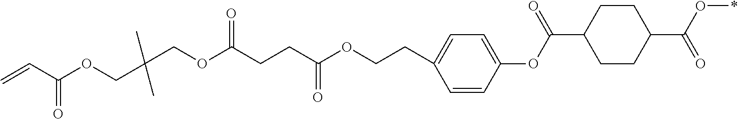 [SMILES: *OC(=O)C1CCC(C(=O)Oc2ccc(CCOC(=O)CCC(=O)OCC(C)(C)COC(=O)C=C)cc2)CC1] |
| 7-2 | 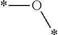 [SMILES: *O*] | 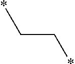 [SMILES: *CC*] | 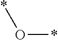 [SMILES: *O*] | 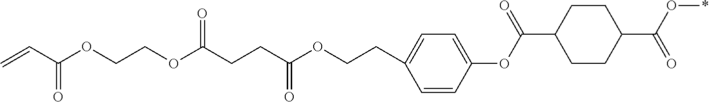 [SMILES: *OC(=O)C1CCC(C(=O)Oc2ccc(CCOC(=O)CCC(=O)OCCOC(=O)C=C)cc2)CC1] |
| 7-3 | 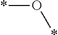 [SMILES: *O*] | 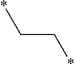 [SMILES: *CC*] | 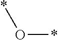 [SMILES: *O*] | 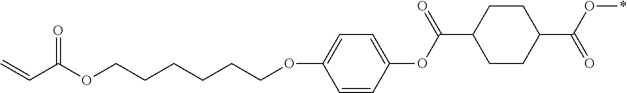 [SMILES: *OC(=O)C1CCC(C(=O)Oc2ccc(OCCCCCCOC(=O)C=C)cc2)CC1] |
| 7-4 | 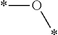 [SMILES: *O*] | 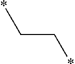 [SMILES: *CC*] | 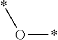 [SMILES: *O*] | 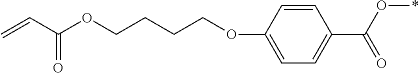 [SMILES: *OC(=O)c1ccc(OCCCCOC(=O)C=C)cc1] |
| 7-5 | 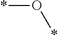 [SMILES: *O*] | 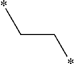 [SMILES: *CC*] | 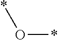 [SMILES: *O*] | 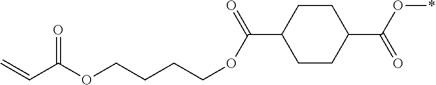 [SMILES: *OC(=O)C1CCC(C(=O)OCCCCOC(=O)C=C)CC1] |
| 7-6 | 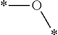 [SMILES: *O*] | 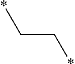 [SMILES: *CC*] | 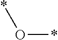 [SMILES: *O*] | 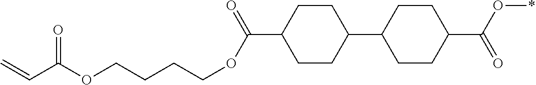 [SMILES: *OC(=O)C1CCC(C2CCC(C(=O)OCCCCOC(=O)C=C)CC2)CC1] |
| 7-7 | 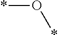 [SMILES: *O*] | 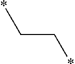 [SMILES: *CC*] | 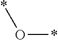 [SMILES: *O*] | 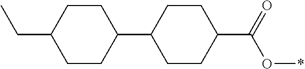 [SMILES: *OC(=O)C1CCC(C2CCC(CC)CC2)CC1] |
| 7-8 | 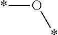 [SMILES: *O*] | 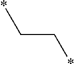 [SMILES: *CC*] | 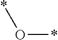 [SMILES: *O*] | 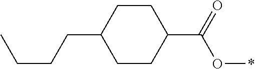 [SMILES: *OC(=O)C1CCC(CCCC)CC1] |
| 7-9 | 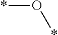 [SMILES: *O*] | 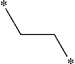 [SMILES: *CC*] | 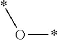 [SMILES: *O*] | 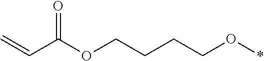 [SMILES: *OCCCCOC(=O)C=C] |
| 7-10 | 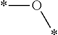 [SMILES: *O*] | 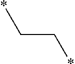 [SMILES: *CC*] | 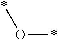 [SMILES: *O*] | 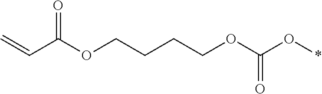 [SMILES: *OC(=O)OCCCCOC(=O)C=C] |

TABLE 8

| | D¹ | G¹ | D² | K |
|---|---|---|---|---|
| 8-1 | 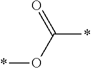 [SMILES: *OC(*)=O] | 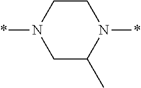 [SMILES: *N1CCN(*)C(C)C1] | 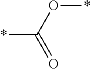 [SMILES: *OC(*)=O] | 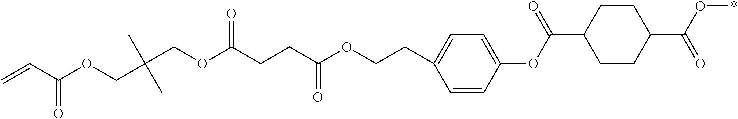 [SMILES: *OC(=O)C1CCC(C(=O)Oc2ccc(CCOC(=O)CCC(=O)OCC(C)(C)COC(=O)C=C)cc2)CC1] |
| 8-2 | 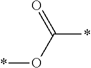 [SMILES: *OC(*)=O] | 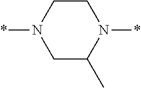 [SMILES: *N1CCN(*)C(C)C1] | 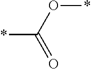 [SMILES: *OC(*)=O] | 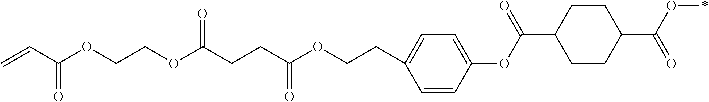 [SMILES: *OC(=O)C1CCC(C(=O)Oc2ccc(CCOC(=O)CCC(=O)OCCOC(=O)C=C)cc2)CC1] |
| 8-3 | 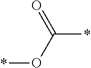 [SMILES: *OC(*)=O] | 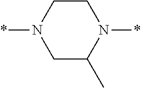 [SMILES: *N1CCN(*)C(C)C1] | 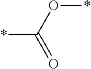 [SMILES: *OC(*)=O] | 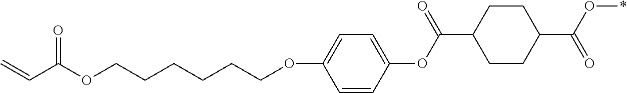 [SMILES: *OC(=O)C1CCC(C(=O)Oc2ccc(OCCCCCCOC(=O)C=C)cc2)CC1] |
| 8-4 | 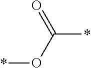 [SMILES: *OC(*)=O] | 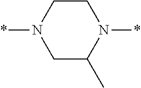 [SMILES: *N1CCN(*)C(C)C1] | 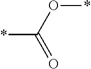 [SMILES: *OC(*)=O] | 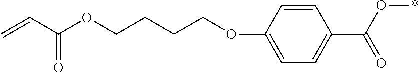 [SMILES: *OC(=O)c1ccc(OCCCCOC(=O)C=C)cc1] |
| 8-5 | 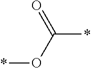 [SMILES: *OC(*)=O] | 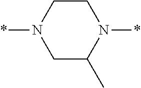 [SMILES: *N1CCN(*)C(C)C1] | 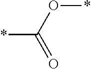 [SMILES: *OC(*)=O] | 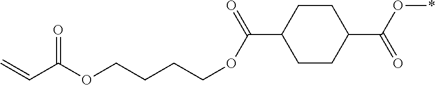 [SMILES: *OC(=O)C1CCC(C(=O)OCCCCOC(=O)C=C)CC1] |
| 8-6 | 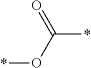 [SMILES: *OC(*)=O] | 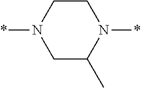 [SMILES: *N1CCN(*)C(C)C1] | 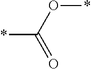 [SMILES: *OC(*)=O] | 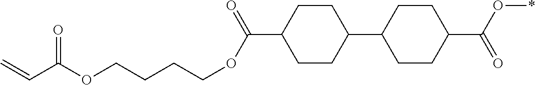 [SMILES: *OC(=O)C1CCC(C2CCC(C(=O)OCCCCOC(=O)C=C)CC2)CC1] |
| 8-7 | 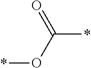 [SMILES: *OC(*)=O] | 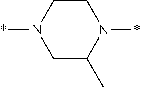 [SMILES: *N1CCN(*)C(C)C1] | 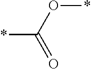 [SMILES: *OC(*)=O] | 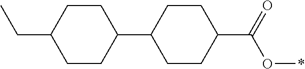 [SMILES: *OC(=O)C1CCC(C2CCC(CC)CC2)CC1] |
| 8-8 | 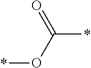 [SMILES: *OC(*)=O] | 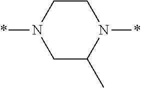 [SMILES: *N1CCN(*)C(C)C1] | 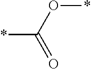 [SMILES: *OC(*)=O] | 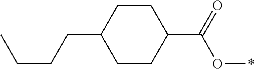 [SMILES: *OC(=O)C1CCC(CCCC)CC1] |
| 8-9 | 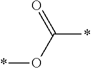 [SMILES: *OC(*)=O] | 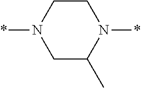 [SMILES: *N1CCN(*)C(C)C1] | 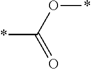 [SMILES: *OC(*)=O] | 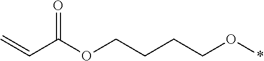 [SMILES: *OCCCCOC(=O)C=C] |
| 8-10 | 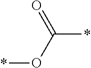 [SMILES: *OC(*)=O] | 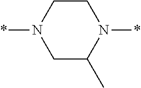 [SMILES: *N1CCN(*)C(C)C1] | 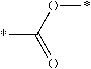 [SMILES: *OC(*)=O] | 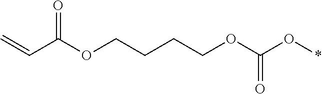 [SMILES: *OC(=O)OCCCCOC(=O)C=C] |

TABLE 9

| | D¹ | G¹ | G¹ | D² | K |
|---|---|---|---|---|---|
| 9-1 | 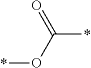 [SMILES: *OC(*)=O] | 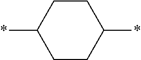 [SMILES: *C1CCC(*)CC1] | 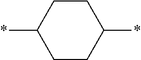 [SMILES: *C1CCC(*)CC1] | 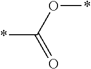 [SMILES: *OC(*)=O] | 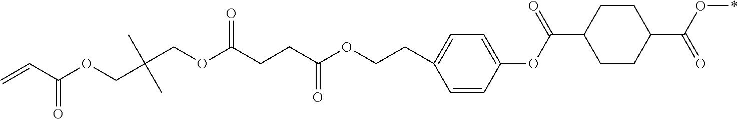 [SMILES: *OC(=O)C1CCC(C(=O)Oc2ccc(CCOC(=O)CCC(=O)OCC(C)(C)COC(=O)C=C)cc2)CC1] |
| 9-2 | 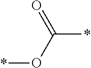 [SMILES: *OC(*)=O] | 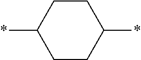 [SMILES: *C1CCC(*)CC1] | 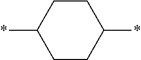 [SMILES: *C1CCC(*)CC1] | 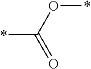 [SMILES: *OC(*)=O] | 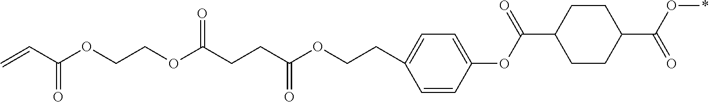 [SMILES: *OC(=O)C1CCC(C(=O)Oc2ccc(CCOC(=O)CCC(=O)OCCOC(=O)C=C)cc2)CC1] |
| 9-3 | 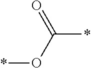 [SMILES: *OC(*)=O] | 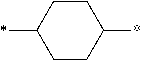 [SMILES: *C1CCC(*)CC1] | 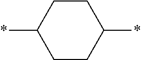 [SMILES: *C1CCC(*)CC1] | 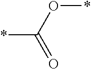 [SMILES: *OC(*)=O] | 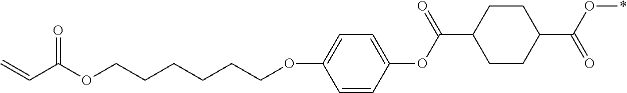 [SMILES: *OC(=O)C1CCC(C(=O)Oc2ccc(OCCCCCCOC(=O)C=C)cc2)CC1] |
| 9-4 | 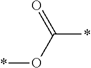 [SMILES: *OC(*)=O] | 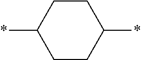 [SMILES: *C1CCC(*)CC1] | 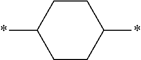 [SMILES: *C1CCC(*)CC1] | 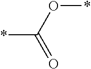 [SMILES: *OC(*)=O] | 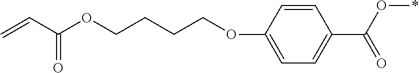 [SMILES: *OC(=O)c1ccc(OCCCCOC(=O)C=C)cc1] |
| 9-5 | 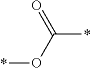 [SMILES: *OC(*)=O] | 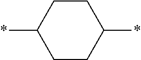 [SMILES: *C1CCC(*)CC1] | 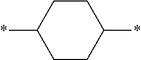 [SMILES: *C1CCC(*)CC1] | 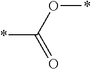 [SMILES: *OC(*)=O] | 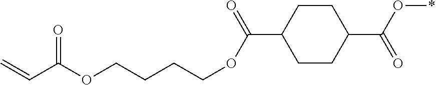 [SMILES: *OC(=O)C1CCC(C(=O)OCCCCOC(=O)C=C)CC1] |
| 9-6 | 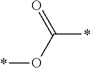 [SMILES: *OC(*)=O] | 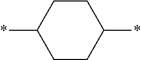 [SMILES: *C1CCC(*)CC1] | 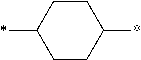 [SMILES: *C1CCC(*)CC1] | 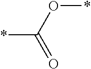 [SMILES: *OC(*)=O] | 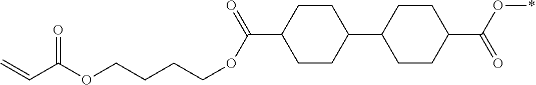 [SMILES: *OC(=O)C1CCC(C2CCC(C(=O)OCCCCOC(=O)C=C)CC2)CC1] |
| 9-7 | 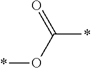 [SMILES: *OC(*)=O] | 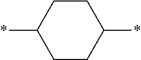 [SMILES: *C1CCC(*)CC1] | 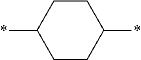 [SMILES: *C1CCC(*)CC1] | 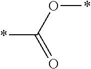 [SMILES: *OC(*)=O] | 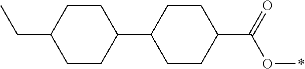 [SMILES: *OC(=O)C1CCC(C2CCC(CC)CC2)CC1] |
| 9-8 | 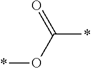 [SMILES: *OC(*)=O] | 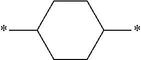 [SMILES: *C1CCC(*)CC1] | 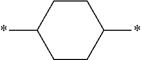 [SMILES: *C1CCC(*)CC1] | 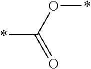 [SMILES: *OC(*)=O] | 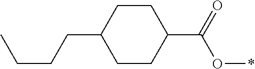 [SMILES: *OC(=O)C1CCC(CCCC)CC1] |
| 9-9 | 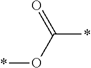 [SMILES: *OC(*)=O] | 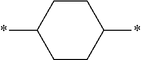 [SMILES: *C1CCC(*)CC1] | 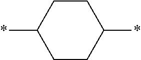 [SMILES: *C1CCC(*)CC1] | 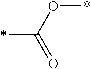 [SMILES: *OC(*)=O] | 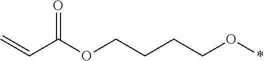 [SMILES: *OCCCCOC(=O)C=C] |
| 9-10 | 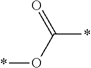 [SMILES: *OC(*)=O] | 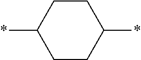 [SMILES: *C1CCC(*)CC1] | 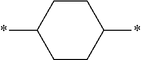 [SMILES: *C1CCC(*)CC1] | 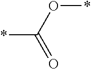 [SMILES: *OC(*)=O] | 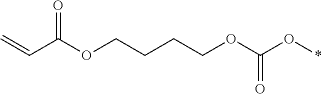 [SMILES: *OC(=O)OCCCCOC(=O)C=C] |

### Second Polymerizable Compound

The second polymerizable compound contained in the liquid crystal composition according to the embodiment of the present invention is a compound represented by Formula (2).

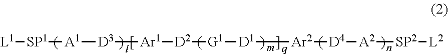

In Formula (2), D¹, D², D³, D⁴, G¹, A¹, A², SP¹, SP², L¹, L², m, l, n, Ar¹, and Ar² each have the same meaning as described in Formula (1), and specific aspects and suitable aspects thereof are also the same.

q represents an integer of 2 to 9, preferably represents an integer of 2 to 5, more preferably represents 2 or 3, and still more preferably represents 2. In Formula (2), a plurality of Ar¹'s may be the same or different from each other, a plurality of D²'s may be the same or different from each other, a plurality of G¹'s may be the same or different from each other, and a plurality of D¹'s may be the same or different from each other.

In the present invention, the content of the second polymerizable compound is 1% by mass or more with respect to the total mass of the first polymerizable compound and the second polymerizable compound; but from the reason that an alignment temperature of the liquid crystal composition is lowered and solubility is increased, the content thereof is preferably 5% to 50% by mass and more preferably 15% to 30% by mass.

### Other Polymerizable Compounds

From the viewpoint of alignment temperature and solubility, it is preferable that the liquid crystal composition according to the embodiment of the present invention contains a polymerizable compound having one or more polymerizable groups, in addition to the above-described first polymerizable compound and second polymerizable compound.

Here, the polymerizable group included in other polymerizable compounds is not particularly limited, and examples thereof include the same as those described for L1 and L2 in Formula (1) above. Among these, an acryloyl group or a methacryloyl group is preferable.

As the other polymerizable compounds, from the reason that the durability of the optically anisotropic film to be formed is more improved, other polymerizable compounds having two to four polymerizable groups are preferable, and other polymerizable compounds having two polymerizable groups are more preferable.

Examples of the other polymerizable compounds include compounds represented by Formulae (M1), (M2), and (M3) described in paragraphs to of JP2014-077068A, which have liquid crystallinity, and more specifically, specific examples described in paragraphs to of the same publication.

### Polymerization Initiator

The liquid crystal composition according to the embodiment of the present invention preferably contains a polymerization initiator.

The polymerization initiator to be used is preferably a photopolymerization initiator capable of initiating a polymerization reaction by irradiation with ultraviolet rays.

Examples of the photopolymerization initiator include a-carbonyl compounds (described in U.S. Pat. No. 2,367,661A and U.S. Pat. No. 2,367,670A), acyloin ethers (described in U.S. Pat. No. 2,448,828A), α-hydrocarbon-substituted aromatic acyloin compounds (described in U.S. Pat. No. 2,722,512A), polynuclear quinone compounds (described in U.S. Pat. No. 3,046,127A and U.S. Pat. No. 2,951,758A), combinations of triarylimidazole dimer and p-aminophenyl ketone (described in U.S. Pat. No. 3,549,367A), acridine and phenazine compounds (described in JP1985-105667A (JP-S60-105667A) and U.S. Pat. No. 4,239,850A), oxadiazole compounds (described in U.S. Pat. No. 4,212,970A), and acyl phosphine oxide compounds (described in JP1988-40799B (JP-S63-40799B), JP1993-29234B (JP-H05-29234B), JP1998-95788A (JP-H10-95788A), and JP1998-29997A (JP-H10-29997A)).

In addition, in the present invention, it is also preferable that the polymerization initiator is an oxime-type polymerization initiator; and specific examples of the polymerization

initiator include initiators described in paragraphs [0049] to [0052] of WO2017/170443A.

### Solvent

From the viewpoint of workability or the like to form the optically anisotropic film, the liquid crystal composition according to the embodiment of the present invention preferably contains a solvent.

Specific examples of the solvent include ketones (for example, acetone, 2-butanone, methyl ethyl ketone, methyl isobutyl ketone, cyclohexanone, cyclopentanone, and the like), ethers (for example, dioxane, tetrahydrofuran, and the like), aliphatic hydrocarbons (for example, hexane and the like), alicyclic hydrocarbons (for example, cyclohexane and the like), aromatic hydrocarbons (for example, toluene, xylene, trimethylbenzene, and the like), halogenated carbons (for example, dichloromethane, dichloroethane, dichlorobenzene, chlorotoluene, and the like), esters (for example, methyl acetate, ethyl acetate, butyl acetate, and the like), water, alcohols (for example, ethanol, isopropanol, butanol, cyclohexanol, and the like), cellosolves (for example, methyl cellosolve, ethyl cellosolve, and the like), cellosolve acetates, sulfoxides (for example, dimethyl sulfoxide and the like), and amides (for example, dimethyl formamide, dimethyl acetamide, and the like), and these solvents may be used alone or in a combination of two or more kinds thereof.

### Leveling Agent

It is preferable that the liquid crystal composition according to the embodiment of the present invention contains a leveling agent from the viewpoint that a surface of a cured substance according to the present invention, which will be described later, is maintained smooth and the alignment is easily controlled.

The leveling agent is preferably a fluorine-based leveling agent or a silicon-based leveling agent for a reason that it has a high leveling effect with respect to the addition amount, and the leveling agent is more preferably a fluorine-based leveling agent from the viewpoint that it is less likely to cause bleeding (bloom or bleed).

Specific examples of the leveling agent include compounds described in paragraphs to of JP2007-069471A, the compound represented by General Formula (I) described in JP2013-047204A (in particular, compounds described in paragraphs [0020] to) [0032]), the compound represented by General Formula (I) described in JP2012-211306A (in

particular, compounds described in paragraphs [0022] to [0029]), the liquid crystal alignment accelerator represented by General Formula (I) described in JP2002-129162A (in particular,

compounds described in paragraphs [0076] to [0078] and [0082] to [0084]), and compounds represented by General Formulae (I), (II), and (III) described in JP2005-099248A (in particular,

compounds described in paragraphs [0082] to [0096]). The leveling agent may also function as an alignment control agent described later.

### Alignment Control Agent

The liquid crystal composition according to the embodiment of the present invention can contain an alignment control agent as necessary.

With the alignment control agent, in addition to the homogeneous alignment, various alignment states such as homeotropic alignment (vertical alignment), tilt alignment, hybrid alignment, and cholesteric alignment can be formed, and specific alignment states can be controlled and achieved more uniformly and more accurately.

As an alignment control agent which accelerates the homogeneous alignment, for example, a low-molecular-weight alignment control agent or a high-molecular-weight alignment control agent can be used.

With regard to the low-molecular-weight alignment control agent, reference can be made to the description in, for example, paragraphs [0009] to [0083] of JP2002-20363A, paragraphs [0011] to [0120] of JP2006-106662A, and paragraphs [0021] [0029] to of JP2012-211306A, the contents of which are incorporated herein by reference.

In addition, with regard to the high-molecular-weight alignment control agent, reference can be made to the description in, for example, paragraphs [0021] to [0057] of JP2004-198511A and paragraphs [0121] to [0167] of JP2006-106662A, the contents of which are incorporated herein by reference.

In addition, examples of the alignment control agent which forms or accelerates the homeotropic alignment include a boronic acid compound and an onium salt compound, and specifically, reference can be made to compounds described in paragraphs [0023] to [0032] of JP2008-225281A, paragraphs [0054] to [0058] of JP2012-208397A, paragraphs [0024] to [0055] of JP2008-026730A, paragraphs [0043] to [0055] of JP2016-193869A, and the like, the contents of which are incorporated herein by reference.

On the other hand, the cholesteric alignment can be achieved by adding a chiral agent to the liquid crystal composition according to the embodiment of the present invention, and it is possible to control the direction of revolution of the cholesteric alignment by its chiral direction.

Incidentally, it is possible to control a pitch of the cholesteric alignment in accordance with an alignment regulating force of the chiral agent.

In a case where an alignment control agent is contained, a content thereof is preferably 0.01% to 10% by mass, and more preferably 0.05% to 5% by mass with respect to the total solid content mass of the composition. In a case where the content is within the range, it is possible to obtain a cured substance which has no precipitation or phase separation, alignment defects, or the like, and is uniform and highly transparent while achieving a desired alignment state.

### Other Components

The liquid crystal composition according to the embodiment of the present invention may contain components other than the above-described components; and examples thereof include a surfactant, a tilt angle control agent, an alignment assistant, a plasticizer, and a crosslinking agent.

### Optically Anisotropic Film

The optically anisotropic film according to the embodiment of the present invention is an optically anisotropic film obtained by fixing an alignment state of the above-described liquid crystal composition according to the embodiment of the present invention.

Examples of a method for forming the optically anisotropic film include a method of using the above-described liquid crystal composition according to the embodiment of the present invention to obtain a desired alignment state, and then fixing an alignment state by polymerization.

Here, polymerization conditions are not particularly limited, but ultraviolet rays are preferably used in the polymerization by light irradiation. An irradiation amount is preferably 10 mJ/cm² to 50 J/cm², more preferably 20 mJ/cm² to 5 J/cm², still more preferably 30 mJ/cm² to 3 J/cm², and particularly preferably 50 to 1,000 mJ/cm². In order to promote the polymerization reaction, the treatment may be performed under heating conditions.

In the present invention, the optically anisotropic film can be formed on any support in the optical film according to the embodiment of the present invention, which will be described later, or on any polarizer in the polarizing plate according to the embodiment of the present invention, which will be described later.

In the present invention, from the viewpoint of improving a contrast ratio in an image display apparatus, particularly in a liquid crystal display device, it is preferable that the optically anisotropic film is a film obtained by aligning the above-described liquid crystal composition according to the embodiment of the present invention to a smectic phase and then polymerizing the liquid crystal composition (fixing the alignment).

This is considered to be because the smectic phase has a higher degree of order than a nematic phase, and scattering caused by the disorder of the alignment of the optically anisotropic film is suppressed. Whether or not the optically anisotropic film exhibits the smectic phase can be determined by whether or not the optically anisotropic film has a periodic structure by X-ray diffraction. For example, the presence or absence of the periodic structure can be confirmed by analyzing a diffraction pattern with a thin film X-ray diffractometer ATXG (manufactured by Rigaku Corporation).

The optically anisotropic film according to the embodiment of the present invention is preferably a positive A-plate or a positive C-plate, and more preferably a positive A-plate.

Here, the positive A-plate and the positive C-plate are defined as follows.

The positive A-plate satisfies a relationship of Expression (A1) and the positive C-plate satisfies a relationship of Expression (C1), assuming that a refractive index in a film in-plane slow axis direction (in a direction in which an in-plane refractive index is maximum) is defined as nx, a refractive index in an in-plane direction orthogonal to the in-plane slow axis is defined as ny, and a refractive index in a thickness direction is defined as nz. The positive A-plate has an Rth showing a positive value and the positive C-plate has an Rth showing a negative value.
Expression (A1) nx>ny≈nz
Expression (C1) nz>nx≈ny

The symbol “≈” encompasses not only a case where both sides are completely the same as each other but also a case where the both sides are substantially the same as each other.

The expression “substantially the same” means that, in the positive A-plate, for example, a case where (ny−nz)×d (in which d is a thickness of a film) is −10 to 10 nm and preferably −5 to 5 nm is also included in “ny≈nz”; and a case where (nx−nz)×d is −10 to 10 nm and preferably −5 to 5 nm is also included in “nx≈nz”. In addition, in the positive C-plate, for example, a case where (nx−ny)×d (in which d is a thickness of a film) is 0 to 10 nm, and preferably 0 to 5 nm is also included in “nx≈ny”.

In a case where the optically anisotropic film according to the embodiment of the present invention is the positive A-plate, from the viewpoint that the retardation layer functions as a λ/4 plate, Re(550) is preferably 100 to 180 nm, more preferably 120 to 160 nm, still more preferably 130 to 150 nm, and particularly preferably 130 to 140 nm.

Here, the “λ/4 plate” is a plate having a λ/4 function, specifically, a plate having a function of converting linearly polarized light having a specific wavelength into circularly polarized light (or converting circularly polarized light into linearly polarized light).

### Optical Film

The optical film according to the embodiment of the present invention is an optical film having the optically anisotropic film according to the embodiment of the present invention.

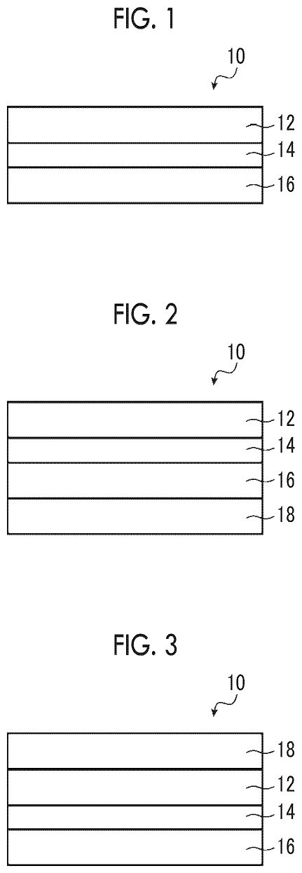

FIGS. 1 to 3 are each a schematic cross-sectional view showing an example of the optical film according to the embodiment of the present invention.

FIGS. 1 to 3 are schematic views, and the thicknesses relationship, the positional relationship, and the like among the respective layers are not necessarily consistent with actual ones, and any of the support, the alignment film, and the hardcoat layer shown in FIGS. 1 to 3 are optional constitutional members.

An optical film 10 shown in FIGS. 1 to 3 has a support 16, an alignment film 14, and an optically anisotropic film 12 in this order.

In addition, the optical film 10 may have a hardcoat layer 18 on a side of the support 16 opposite to a side on which the alignment film 14 is provided as shown in FIG. 2, or may have a hardcoat layer 18 on a side of the optically anisotropic film 12 opposite to a side on which the alignment film 14 is provided as shown in FIG. 3.

Hereinafter, various members used for the optical film according to the embodiment of the present invention will be described in detail.

### Optically Anisotropic Film

The optically anisotropic film included in the optical film according to the embodiment of the present invention is the above-described optically anisotropic film according to the embodiment of the present invention.

In the optical film according to the embodiment of the present invention, a thickness of the above-described optically anisotropic film is not particularly limited, but is preferably 0.1 to 10 μm and more preferably 0.5 to 5 μm.

### Support

The optical film according to the embodiment of the present invention may have a support as a substrate for forming the optically anisotropic film as described above.

Such a support is preferably transparent, and specifically, the support preferably has a light transmittance of 80% or more.

Examples of such a support include a glass substrate and a polymer film; and examples of a material of the polymer film include cellulose-based polymers; acrylic polymers having an acrylic acid ester polymer such as polymethyl methacrylate and a lactone ring-containing polymer; thermoplastic norbornene-based polymers; polycarbonate-based polymers; polyester-based polymers such as polyethylene terephthalate and polyethylene naphthalate; styrene-based polymers such as polystyrene and an acrylonitrile-styrene copolymer (AS resin); polyolefin-based polymers such as polyethylene, polypropylene, and an ethylene-propylene copolymer; vinyl chloride-based polymers; amide-based polymers such as nylon and aromatic polyamide; imide-based polymers; sulfone-based polymers; polyether sulfone-based polymers; polyether ether ketone-based polymers; polyphenylene sulfide-based polymers; vinylidene chloride-based polymers; vinyl alcohol-based polymers; vinyl butyral-based polymers; arylate-based polymers; polyoxymethylene-based polymers; epoxy-based polymers; and polymers obtained by mixing these polymers.

In addition, an aspect in which a polarizer described later may also function as the support is also adopted.

In the present invention, a thickness of the above-described support is not particularly limited, but is preferably 5 to 60 μm and more preferably 5 to 30 μm.

### Alignment Film

In a case where the optical film according to the embodiment of the present invention has any of the above-described supports, it is preferable that the optical film has an alignment film between the support and the optically anisotropic film. An aspect in which the above-described support may also function as the alignment film is also adopted.

The alignment film generally contains a polymer as a main component. A polymer material for an alignment film is described in many documents, and many commercially available products can be used.

The polymer material used in the present invention is preferably polyvinyl alcohol, polyimide, or derivatives thereof. Modified or unmodified polyvinyl alcohol is particularly preferable.

Examples of the alignment film which can be used in the present invention include alignment films described in Line 24 on Page 43 to Line 8 on Page 49 of WO01/88574A; modified polyvinyl alcohols described in paragraphs [0071] to [0095] of JP3907735B; and liquid crystal alignment film formed by a liquid crystal aligning agent, described in JP2012-155308A.

In the present invention, for the reason that it is possible to prevent deterioration in the surface condition by avoiding a contact with the surface of the alignment film upon formation of the alignment film, a photo-alignment film is also preferably used as the alignment film.

The photo-alignment film is not particularly limited, but polymer materials such as a polyamide compound and a polyimide compound, described in paragraphs [0024] to [0043] of WO2005/096041A; liquid crystal alignment film formed by a liquid crystal aligning agent having a photo-aligned group, described in JP2012-155308A; trade name LPP-JP265CP manufactured by Rolic Technologies Ltd.; or the like can be used.

In addition, in the present invention, a thickness of the above-described alignment film is not particularly limited; but from the viewpoint of forming an optically anisotropic film having a homogeneous film thickness by alleviating the surface roughness which can be present on the support, the thickness thereof is preferably 0.01 to 10 μm, more preferably 0.01 to 1 μm, and still more preferably 0.01 to 0.5 μm.

### Hardcoat Layer

It is preferable that the optical film according to the embodiment of the present invention has a hardcoat layer in order to impart physical strength to the film. Specifically, the optical film may have the hardcoat layer on the side of the support opposite to the side on which the alignment film is provided (see FIG. 2), or may have the hardcoat layer on the side of the optically anisotropic film opposite to the side on which the alignment film is provided (see FIG. 3).

As the hardcoat layer, those described in paragraphs [0190] to [0196] of JP2009-98658A can be used.

### Other Optically Anisotropic Films

The optical film according to the embodiment of the present invention may have other optically anisotropic films, in addition to the optically anisotropic film according to the embodiment of the present invention.

That is, the optical film according to the embodiment of the present invention may have a laminated structure of the optically anisotropic film according to the embodiment of the present invention and other optically anisotropic films.

The other optically anisotropic films are not particularly limited as long as they are an optically anisotropic film obtained by using the above-described other polymerizable compounds (particularly, liquid crystal compounds) without blending the first polymerizable compound represented by Formula (1) and/or the second polymerizable compound represented by Formula (2) described above.

Here, in general, the liquid crystal compound can be classified into a rod-like type and a disk-like type according to the shape thereof. Furthermore, each type includes a low molecular type and a polymer type. The term high molecular generally refers to a compound having a degree of polymerization of 100 or more (Polymer Physics-Phase Transition Dynamics, written by Masao Doi, p. 2, published by Iwanami Shoten, 1992). In the present invention, any liquid crystal compound can be used, and it is preferable to use a rod-like liquid crystal compound or a discotic liquid crystal compound (disk-like liquid crystal compound). A mixture of two or more kinds of the rod-like liquid crystal compounds, a mixture of two or more kinds of the disk-like liquid crystal compounds, or a mixture of the rod-like liquid crystal compound and the disk-like liquid crystal compound may be used. In order to immobilize the above-described liquid crystal compound, it is more preferable that the optically anisotropic film is formed of a rod-like liquid crystal compound or disk-like liquid crystal compound having a polymerizable group, and it is still more preferable that the liquid crystal compound has two or more polymerizable groups in one molecule. In the case of th mixture of two or more kinds of liquid crystal compounds, at least one kind of the liquid crystal compound preferably has two or more polymerizable groups in one molecule.

As the rod-like liquid crystal compound, for example, rod-like liquid crystal compounds described in claim 1 of JP1999-513019A (JP-H11-513019A) or paragraphs 0026 to 0098 of JP2005-289980A can be preferably used; and as the discotic liquid crystal compounds, for example, discotic liquid crystal compounds described in paragraphs 0020 to 0067 of JP2007-108732A or paragraphs 0013 to 0108 of JP2010-244038A can be preferably used, but the liquid crystal compounds are not limited thereto.

### Polarizing Plate

The polarizing plate according to the embodiment of the present invention includes the above-described optical film according to the embodiment of the present invention and a polarizer.

In addition, in a case where the above-described optically anisotropic film according to the embodiment of the present invention is a λ/4 plate (positive A-plate), the polarizing plate according to the embodiment of the present invention can be used as a circularly polarizing plate.

In addition, in the polarizing plate according to the embodiment of the present invention, in a case where the above-described optically anisotropic film according to the embodiment of the present invention is a λ/4 plate (positive A-plate), an angle between a slow axis of the λ/4 plate and an absorption axis of the polarizer, which will be described later, is preferably 30° to 60°, more preferably 40° to 50°, still more preferably 42° to 48°, and particularly preferably 45°.

Here, the “slow axis” of the λ/4 plate means a direction in which a refractive index in the plane of the λ/4 plate is maximum, and the “absorption axis” of the polarizer means a direction in which an absorbance is highest.

### Polarizer

The polarizer of the polarizing plate according to the embodiment of the present invention is not particularly limited as long as the polarizer is a member having a function of converting light into specific linearly polarized light, and a known absorptive type polarizer and reflective type polarizer in the related art can be used.

An iodine-based polarizer, a dye-based polarizer using a dichroic dye, a polyene-based polarizer, or the like is used as the absorptive type polarizer. The iodine-based polarizer and the dye-based polarizer include a coating type polarizer and a stretching type polarizer, and any one of these polarizers can be applied. However, a polarizer which is produced by allowing polyvinyl alcohol to adsorb iodine or a dichroic dye and performing stretching is preferable.

In addition, examples of a method of obtaining a polarizer by performing stretching and dyeing in a state of a laminated film in which a polyvinyl alcohol layer is formed on a substrate include methods disclosed in JP5048120B, JP5143918B, JP4691205B, JP4751481B, and JP4751486B, and known technologies related to these polarizers can be preferably used.

A polarizer in which thin films having different birefringence are laminated, a wire grid type polarizer, a polarizer in which a cholesteric liquid crystal having a selective reflection range and a ¼ wavelength plate are combined, or the like is used as the reflective type polarizer.

Among these, from the viewpoint of more excellent adhesiveness, a polarizer containing a polyvinyl alcohol-based resin (polymer including —CH₂—CHOH— as a repeating unit; in particular, at least one selected from the group consisting of polyvinyl alcohol and an ethylene-vinyl alcohol copolymer) is preferable.

In the present invention, a thickness of the polarizer is not particularly limited, but is preferably 3 μm to 60 μm, more preferably 5 μm to 30 μm, and still more preferably 5 μm to 15 μm.

### Pressure Sensitive Adhesive Layer

The polarizing plate according to the embodiment of the present invention may include a pressure sensitive adhesive layer arranged between the optically anisotropic film in the optical film according to the embodiment of the present invention and the polarizer.

The pressure sensitive adhesive layer used for lamination of the optically anisotropic film and the polarizer is, for example, a substance in which a ratio (tanδ=G″/G′) of loss elastic modulus G″ to storage elastic modulus G′ is 0.001 to 1.5, where G′ and G″ are measured with a dynamic viscoelasticity measurement. Such a substance includes a so-called pressure sensitive adhesive or easily creepable substance. Examples of the pressure sensitive adhesive which can be used in the present invention include a polyvinyl alcohol-based pressure sensitive adhesive, but the gluing agent is not limited thereto.

### Image Display Apparatus

An image display apparatus according to an embodiment of the present invention is an image display apparatus having the optical film according to the embodiment of the present invention or the polarizing plate according to the embodiment of the present invention.

A display element used for the image display apparatus according to the embodiment of the present invention is not particularly limited, and examples thereof include a liquid crystal cell, an organic electroluminescent (hereinafter, abbreviated as “EL”) display panel, and a plasma display panel.

Among these, a liquid crystal cell or an organic EL display panel is preferable, and a liquid crystal cell is more preferable. That is, the image display apparatus according to the embodiment of the present invention is preferably a liquid crystal display device using a liquid crystal cell as the display element or an organic EL display device using an organic EL display panel as the display element, and is more preferably a liquid crystal display device.

### Liquid Crystal Display Device

A liquid crystal display device as an example of the image display apparatus according to the embodiment of the present invention is a liquid crystal display device including the polarizing plate according to the embodiment of the present invention and a liquid crystal cell.

In the present invention, it is preferable that the polarizing plate according to the embodiment of the present invention is used for a polarizing plate of the front side, out of polarizing plates provided on both sides of the liquid crystal cell, and it is more preferable that the polarizing plate according to the embodiment of the present invention is used for the polarizing plates on the front and rear sides.

Hereinafter, the liquid crystal cell constituting the liquid crystal display device will be described in detail.

### Liquid Crystal Cell

The liquid crystal cell used for the liquid crystal display device is preferably a vertical alignment (VA) mode, an optically compensated bend (OCB) mode, an in-plane-switching (IPS) mode, or a twisted nematic (TN) mode, but the liquid crystal cell is not limited thereto.

In the liquid crystal cell in a TN mode, rod-like liquid crystalline molecules are substantially horizontally aligned at the time of no voltage application and further twisted aligned at 60° to 120°. The liquid crystal cell in a TN mode is most frequently used as a color TFT liquid crystal display device and is described in a plurality of documents.

In the liquid crystal cell in a VA mode, rod-like liquid crystalline molecules are substantially vertically aligned at the time of no voltage application. The concept of the liquid crystal cell in a VA mode includes (1) a liquid crystal cell in a VA mode in a narrow sense where rod-like liquid crystalline molecules are aligned substantially vertically at the time of no voltage application and substantially horizontally at the time of voltage application (described in JP1990-176625A (JP-H2-176625A)), (2) a liquid crystal cell (in an MVA mode) (SID97, described in Digest of tech. Papers (proceedings) 28 (1997) 845) in which the VA mode is formed to have multi-domain in order to expand the viewing angle, (3) a liquid crystal cell in a mode (n-ASM mode) in which rod-like liquid crystalline molecules are substantially vertically aligned at the time of no voltage application and twistedly multi-domain aligned at the time of voltage application (described in proceedings of Japanese Liquid Crystal Conference, pp. 58 to 59 (1998)), and (4) a liquid crystal cell in a SURVIVAL mode (presented at LCD International 98). In addition, the liquid crystal cell may be of any of a patterned vertical alignment (PVA) type, a photo-alignment (optical alignment) type, or a polymer-sustained alignment (PSA). The details of these modes are described in JP2006-215326A and JP2008-538819A.

In an IPS mode liquid crystal cell, rod-like liquid crystal molecules are substantially aligned parallel to a substrate and application of an electric field parallel to a surface of the substrate causes the liquid crystal molecules to respond planarly. In the IPS mode, black display is carried out in a state where no electric field is applied, and absorption axes of a pair of upper and lower polarizing plates are orthogonal to each other. A method of reducing light leakage during black display in an oblique direction and improve the viewing angle using an optical compensation sheet is disclosed in JP1998-54982A (JP-H10-54982A), JP1999-202323A (JP-H11-202323A), JP1997-292522A (JP-H9-292522A), JP1999-133408A (JP-H11-133408A), JP1999-305217A (JP-H11-305217A), and JP1998-307291A (JP-H10-307291A).

### Organic EL Display Device

Suitable examples of the organic EL display device which is an example of the image display apparatus according to the embodiment of the present invention include an aspect which includes, from a viewing side, a polarizer, a λ/4 plate (a positive A-plate) including the optically anisotropic film according to the embodiment of the present invention, and an organic EL display panel in this order.

In addition, the organic EL display panel is a display panel formed of an organic EL element obtained by sandwiching an organic light emitting layer (organic electroluminescence layer) between electrodes (between a cathode and an anode). The configuration of the organic EL display panel is not particularly limited, and a known configuration is employed. Examples

Hereinafter, the present invention will be described in more detail with reference to Examples. The materials, amounts used, proportions, treatment contents, treatment procedures, and the like shown in the following examples can be modified as appropriate in the range of not departing from the spirit of the present invention. Therefore, the scope of the present invention should not be construed as being limited to Examples.

### Example 1

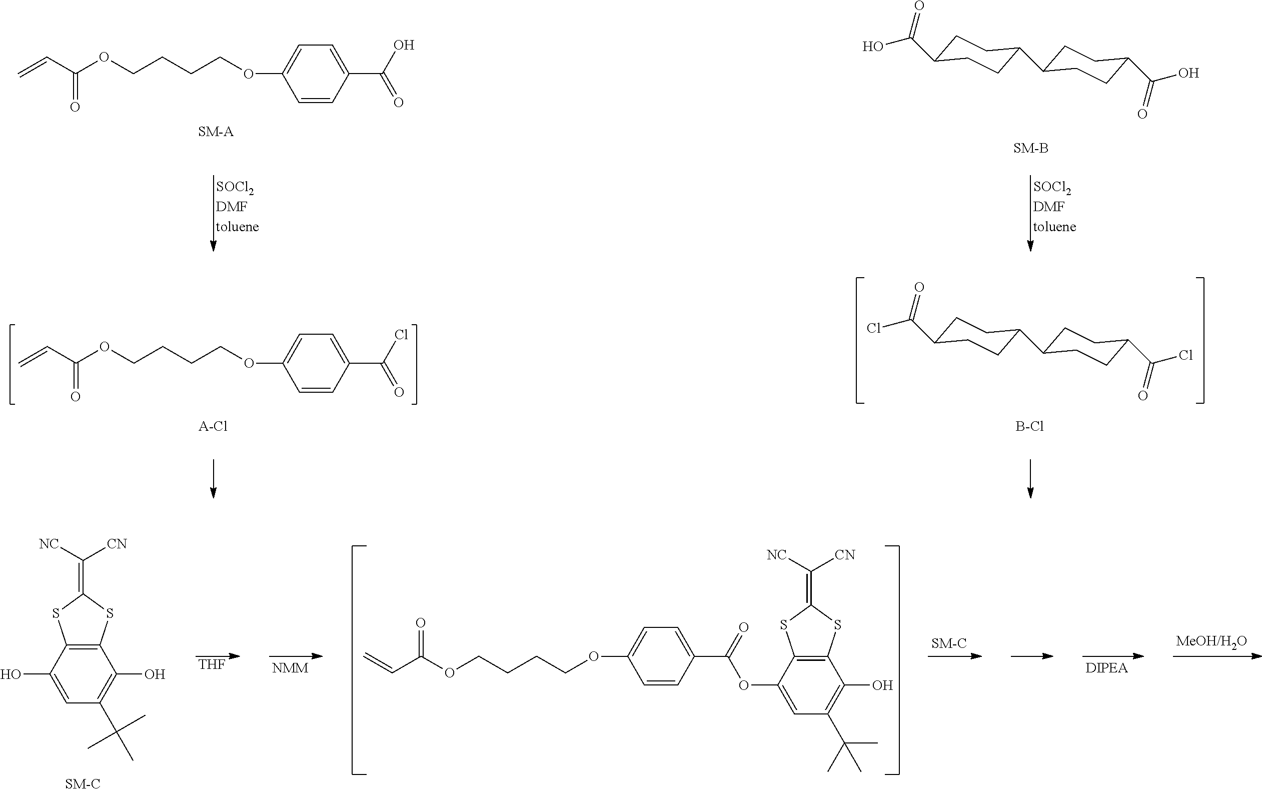

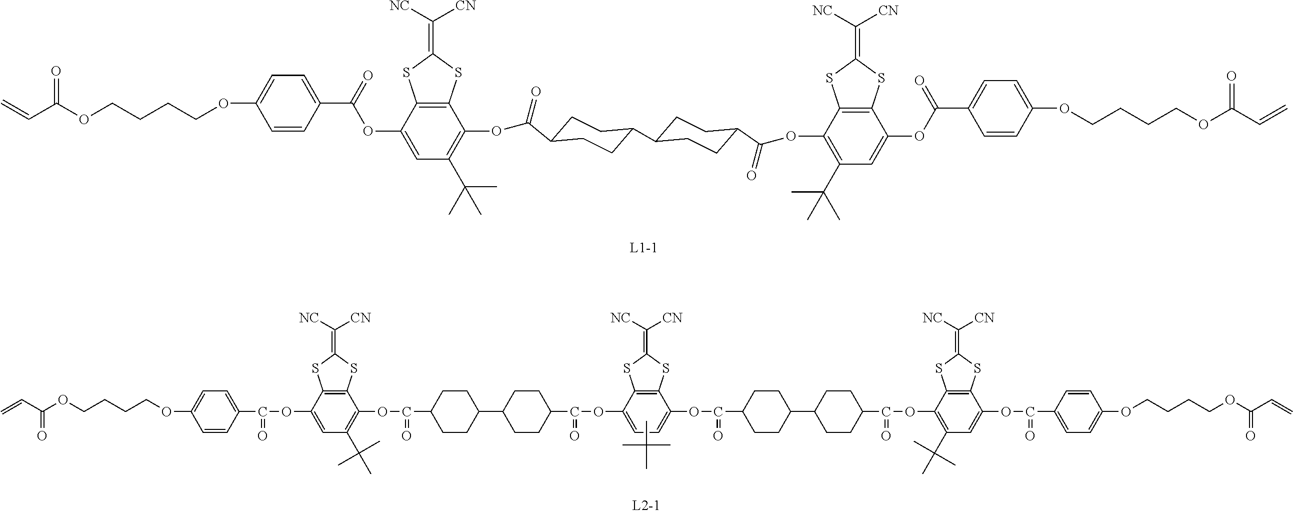

### (1) Synthesis 1 of Acid Chloride

5.21 g (19.2 mmol) of 4-(4-acryloxy-butyl-1-oxy)-benzoic acid (SM-A), 5.21 ml of toluene, and 1.83 ml of dehydrated N,N-dimethylformamide (DMF) were charged into a 50 mL three-neck flask, and the mixture was stirred.

Next, 2.58 g (21.7 mmol) of thionyl chloride was added dropwise thereto under ice cooling, and the mixture was stirred at room temperature for 1 hour after the dropwise addition. Thereafter, the stirring was stopped and the mixture was allowed to stand to remove the separated lower layer, thereby obtaining an acid chloride solution (A-Cl) of SM-A.

### (2) Synthesis 2 of Acid Chloride

2.09 g (8.2 mmol) of (SM-B), 4.00 ml of toluene, and 1.53 ml of dehydrated N,N-dimethylformamide (DMF) were charged into a 50 mL three-neck flask, and the mixture was stirred.

Next, 2.15 g (18.1 mmol) of thionyl chloride was added dropwise thereto under ice cooling, and the mixture was stirred at room temperature for 1 hour after the dropwise addition. Thereafter, the stirring was stopped and the mixture was allowed to stand to remove the separated lower layer, thereby obtaining an acid chloride solution (B-Cl) of SM-B.

### (3) Esterification

5.00 g (16.4 mmol) of 2-(5-tert-butyl-4,7-dihydroxy-1,3-benzodithiol-2-ylidene)propanedinitrile (SM-C) and 50 ml of tetrahydrofuran (THF) were charged into a 200 mL three-neck flask, and the mixture was stirred under ice cooling.

Next, the acid chloride solution (A-Cl) prepared as described above was added dropwise thereto while maintaining an internal temperature of lower than 15° C., and then 2.99 g (21.7 mmol) of N-methylmorpholine (NMM) was added dropwise thereto while maintaining an internal temperature of lower than 10° C. After completion of the dropwise addition, the mixture was continuously stirred at an internal temperature of 7° C. for 1 hour. 0.35 g (1.1 mmol) of the additional amount of SM-C was added to the reaction mixture, the acid chloride solution (B-Cl) was added dropwise thereto while maintaining an internal temperature of lower than 10° C., and then 4.46 g (34.5 mmol) of N,N-diisopropylethylamine (DIPEA) was added dropwise thereto while maintaining an internal temperature of lower than 13° C. After completion of the dropwise addition, the reaction solution was heated to room temperature, and then continuously stirred for 2 hours.

Thereafter, a mixed solution of 10 ml of water and 40 ml of methanol was added to the reaction solution, and the precipitated crystals were collected by suction filtration and washed with 20 ml of methanol. The obtained crystals were blast-dried at 40° C. overnight to obtain 7.17 g (yield: 67%, light yellow crystals) of a composition containing a first polymerizable compound (L1-1) represented by Formula L1-1 above and a second polymerizable compound (L2-1) represented by Formula L2-1 above. In a case where a formulation of the obtained composition was analyzed by high performance liquid chromatography (HPLC), the composition was a mixture containing 88% of the first polymerizable compound (L1-1) and 12% of the second polymerizable compound (L2-1) (area % ratio at 254 nm).

The results of analyzing structures of the first polymerizable compound (L1-1) and the second polymerizable compound (L2-1), which are isolated, by ¹H-nuclear magnetic resonance (NMR) are shown below.

### First Polymerizable Compound (L1-1)

¹H-NMR (solvent: CDCl₃) δ (ppm): 8.13 (4H, d), 7.43 (2H, s), 7.01 (4H, d), 6.42 (2H, d), 6.09 to 6.18 (2H, m), 5.85 (2H, d), 4.27 (4H, m), 4.12 (4H, m), 2.60 (2H, m), 2.25 (4H, d), 1.85 to 2.00 (12H, m), 1.55 to 1.72 (4H, m), 1.38 (18H, s), 1.11 to 1.31 (8H, m)

### Second Polymerizable Compound (L2-1)

¹H-NMR (solvent: CDCl₃) δ (ppm): 8.13 (4H, d), 7.43 (2H, s), 7.30 (1H, s), 7.01 (4H, d), 6.42 (2H, d), 6.09 to 6.18 (2H, m), 5.85 (2H, d), 4.27 (4H, m), 4.12 (4H, m), 2.60 (4H, m), 2.25 (4H, d), 1.85 to 2.00 (20H, m), 1.55 to 1.72 (8H, m), 1.38 (18H, s), 1.37 (9H, s), 1.11 to 1.31 (16H, m)

### Example 2

A liquid crystal composition 2 was prepared by the same method as in Example 1, except that, in the esterification, the additional amount of SM-C was changed to 0.70 g (2.3 mmol). Ratios of the first polymerizable compound (L1-1) and the second polymerizable compound (L2-1) contained in the liquid crystal composition 2 were 80% and 20%, respectively.

The liquid crystal composition 2 prepared in Example 2 also included a tetrakis-form and a pentakis-form. A content of the tetrakis-form was 4% with respect to the total amount of the first polymerizable compound (L1-1) and the second polymerizable compound (L2-1), and a content of the pentakis-form was 1% with respect to the total amount of the first polymerizable compound (L1-1) and the second polymerizable compound (L2-1).

### Example 3

Other polymerizable compound (L3-1) represented by Formula L3-1 below was blended with the liquid crystal composition 2 prepared in Example 2 at a mass ratio [Liquid crystal composition 2/polymerizable compound shown below=84/16] to prepare a liquid crystal composition 3.

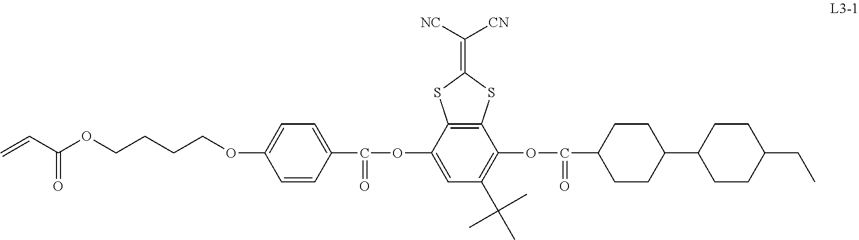 [SMILES: C=CC(=O)OCCCCOc1ccc(C(=O)Oc2cc(C(C)(C)C)c(OC(=O)C3CCC(C4CCC(CC)CC4)CC3)c3c2SC(=C(C#N)C#N)S3)cc1]

The other polymerizable compound (L3-1) was synthesized by a method shown below.

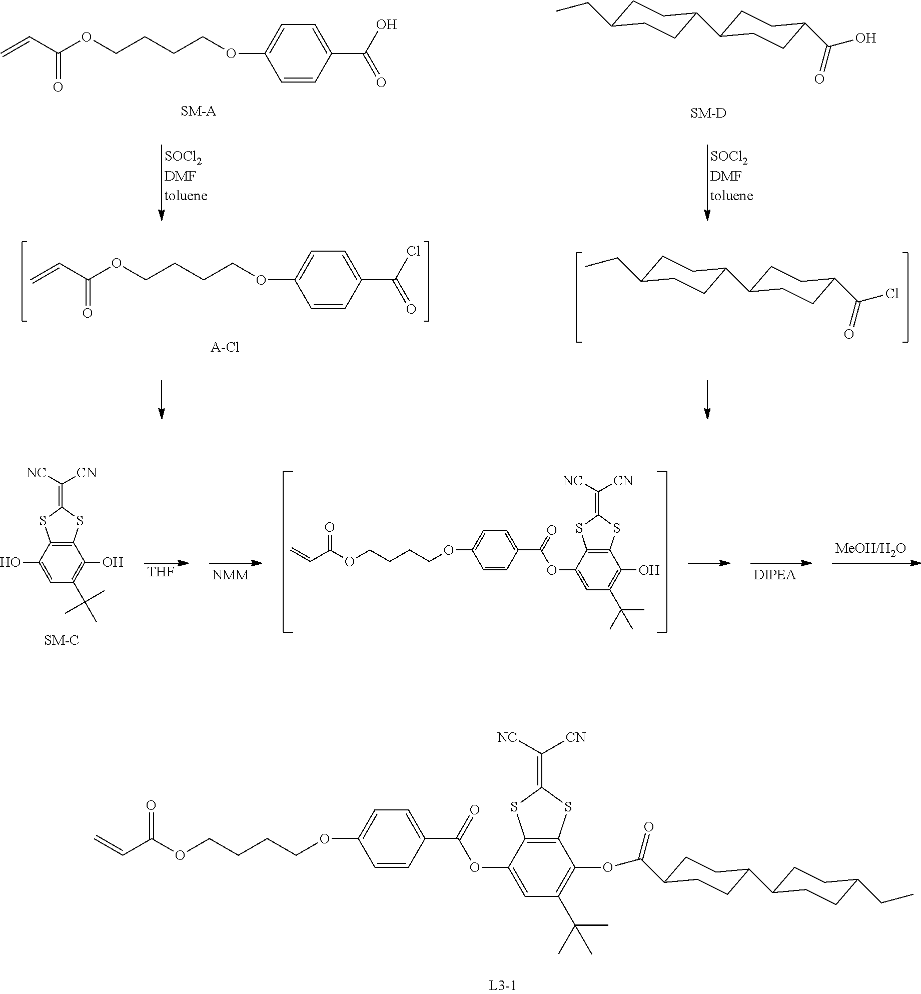

### Synthesis 3 of Acid Chloride

3.91 g (16.4 mmol) of (SM-D), 4.001 ml of toluene, and 1.53 ml of dehydrated N,N-dimethylformamide (DMF) were charged into a 50 mL three-neck flask, and the mixture was stirred.

Next, 2.15 g (18.1 mmol) of thionyl chloride was added dropwise thereto under ice cooling, and the mixture was stirred at room temperature for 1 hour after the dropwise addition. Thereafter, the stirring was stopped and the mixture was allowed to stand to remove the separated lower layer, thereby obtaining an acid chloride solution (D-Cl) of SM-D.

### Synthesis of Other Polymerizable Compound (L3-1)

The other polymerizable compound (L3-1) was synthesized by the same method as in the esterification in Example 1, except that, in the reaction of esterification in Example 1, the additional amount of SM-C was not added and the acid chloride solution (D-Cl) was used instead of the acid chloride solution (B-Cl).

The result of analyzing a structure of the other polymerizable compound (L3-1) by ¹H-NMR is shown below.

¹H-NMR (solvent: CDCl₃) δ (ppm): 8.13 (2H, d), 7.43 (1H, s), 7.01 (2H, d), 6.42 (1H, d), 6.09 to 6.18 (1H, m), 5.85 (1H, d), 4.27 (2H, m), 4.12 (2H, m), 2.60 (1H, m), 2.25 (2H, d), 1.85 to 2.00 (6H, m), 1.72 to 1.84 (4H, m), 1.55 to 1.70 (2H, m), 1.38 (9H, s), 0.92 to 1.25 (11H, m), 0.88 (3H, t)

### Example 4

A liquid crystal composition 4 was prepared by the same method as in Example 1, except that, in the esterification, the additional amount of SM-C was not blended. Ratios of the first polymerizable compound (L1-1) and the second polymerizable compound (L2-1) contained in the liquid crystal composition 4 were 98% and 2%, respectively.

### Example 5

A liquid crystal composition 5 containing a first polymerizable compound (L1-2) represented by Formula L1-2 above and a second polymerizable compound (L2-2) represented by Formula L2-2 above was prepared by the same method as in Example 1, except that, in the esterification, the acid chloride solution (B-Cl) was changed to 1.71 g (8.2 mmol) of trans-cyclohexane-1,4-dicarbonyl dichloride. Ratios of the first polymerizable compound (L1-2) and the second polymerizable compound (L2-2) contained in the liquid crystal composition 5 were 89% and 11%. respectively.

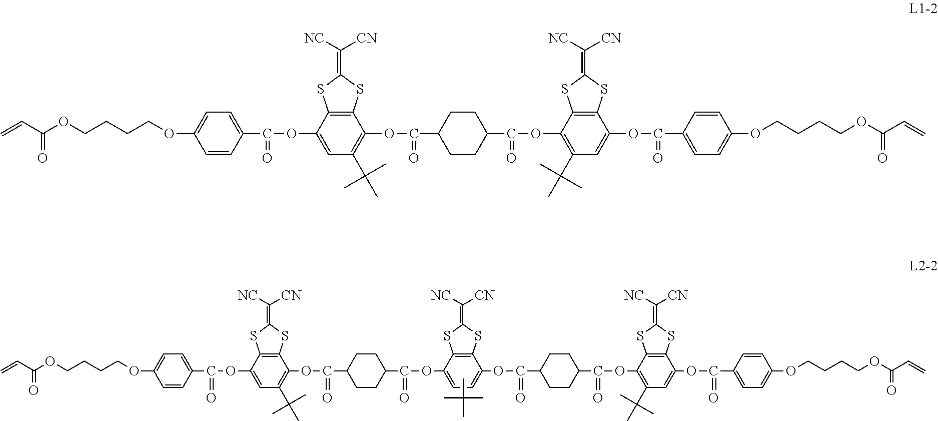

### Example 6

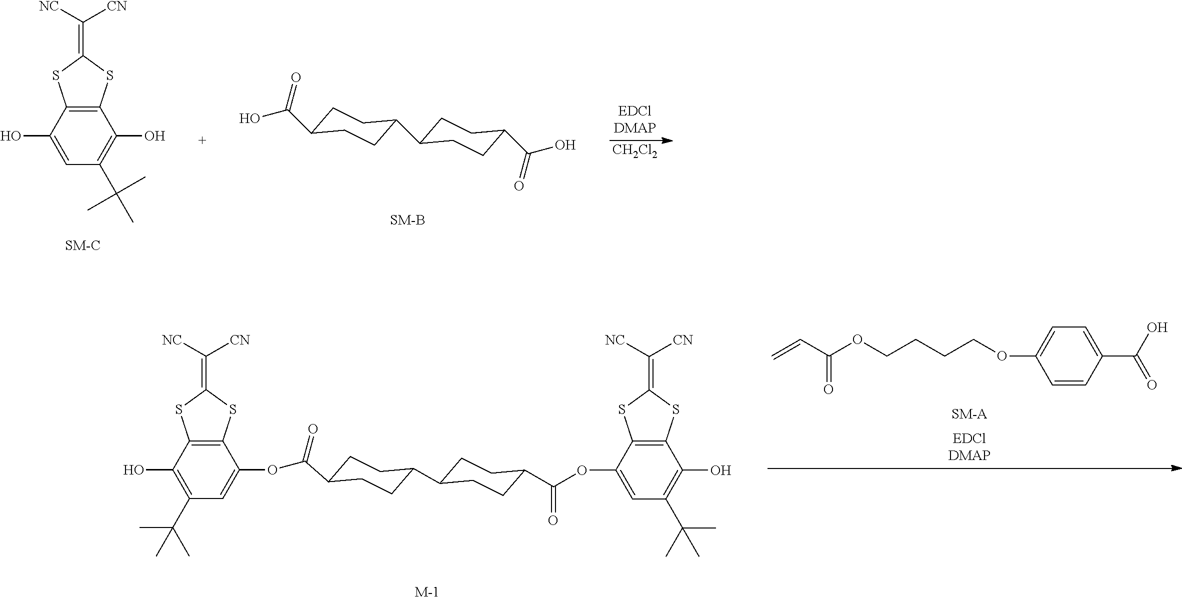

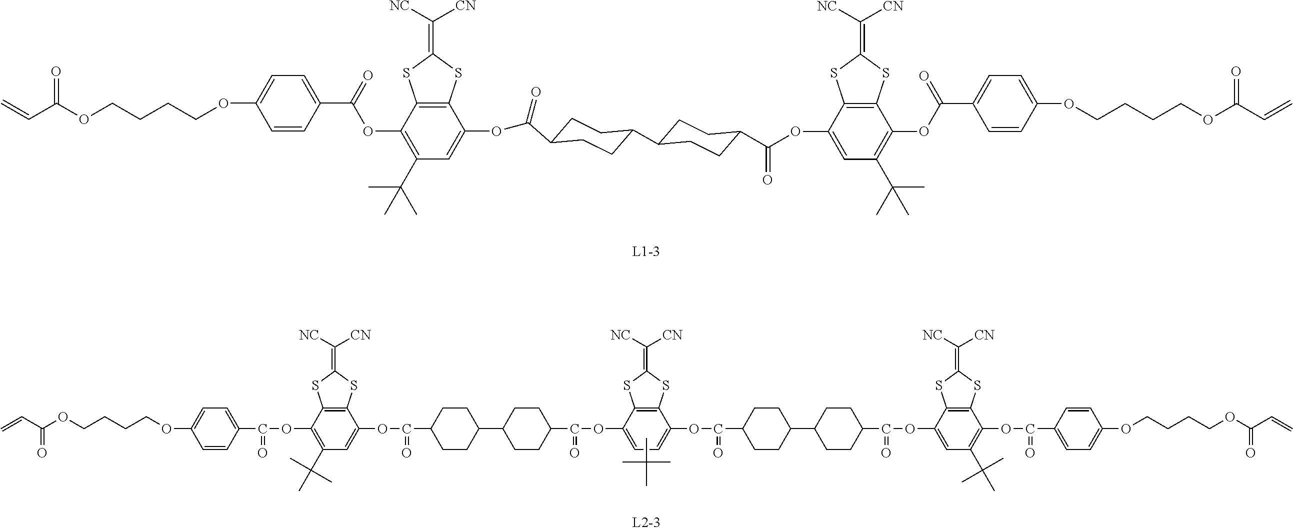

(1) Synthesis of intermediate M-1

12.30 g (40.3 mmol) of SM-C, 19.7 mmol of 4-(4-carboxycyclohexyl)cyclohexanecarboxylic acid, and 50 ml of dichloromethane were charged into a 200 mL three-neck flask, 11.3 g (59.0 mmol) of 3-(ethyliminomethylamino)-N,N-dimethyl-propan-1-amine; hydrochloride and 0.49 g (3.9 mmol) of 4-dimethylaminopyridine were added thereto, and the mixture was stirred at room temperature for 3 hours. 100 ml of water was added to the reaction solution, the mixture was stirred, the aqueous layer was removed, and 200 ml of ethanol was added to the mixture to precipitate solid which was collected by suction filtration. The filtered solid was blast-dried overnight at room temperature to obtain 18.4 g of a target intermediate M-1 (yield: 84%, light yellow crystals).

### (2) Preparation of Liquid Crystal Composition 6

1.0 g (1.21 mmol) of the intermediate M-1, 0.66 g (2.48 mmol) of SM-A, and 10 mL of dichloromethane were charged into a 100 mL three-neck flask, 0.70 g (3.63 mmol) of 3-(ethyliminomethyleneamino)-N,N-dimethyl-propan-1-amine; hydrochloride and 30 mg (0.24 mmol) of 4-dimethylaminopyridine were added thereto, and the mixture was stirred at room temperature for 2 hours. 20 ml of water was added to the reaction solution and stirred, the aqueous layer was removed, and 20 ml of methanol was added to the reaction solution to precipitate solid which was collected by suction filtration. The filtered solid was blast-dried overnight at room temperature to prepare 60.48 g of a liquid crystal composition containing a polymerizable compound (L1-3) represented by Formula L1-3 above and a polymerizable compound (L2-3) represented by Formula L2-3 above. Ratios of the first polymerizable compound (L1-3) and the second polymerizable compound (L2-3) contained in the liquid crystal composition 6 were 88% and 12%, respectively.

### Example 7

A liquid crystal composition 7 containing a polymerizable compound (L1-4) represented by Formula L1-4 above and a polymerizable compound (L2-4) represented by Formula L2-4 above was prepared by the same method as in Example 6, except that 1,4-cyclohexanedicarboxylic acid was used instead of the 4-(4-carboxycyclohexyl)cyclohexanecarboxylic acid. Ratios of the first polymerizable compound (L1-4) and the second polymerizable compound (L2-4) contained in the liquid crystal composition 7 were 88% and 12%, respectively.

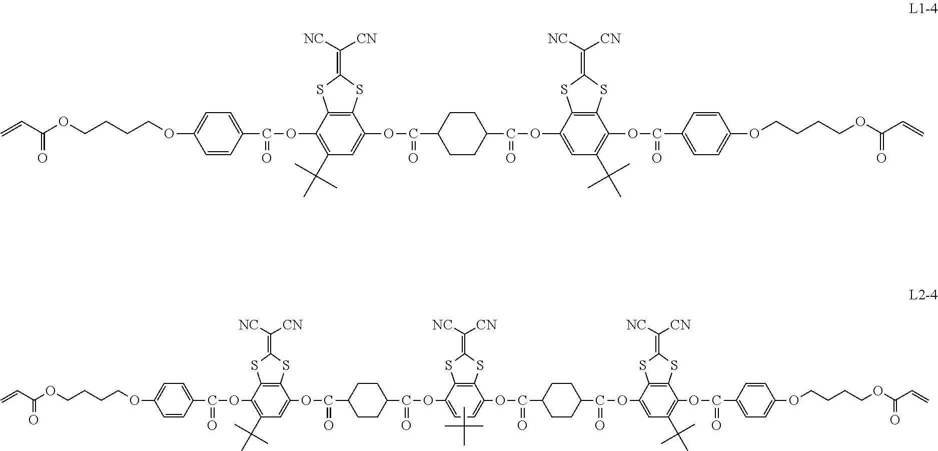

### Example 8

A liquid crystal composition 8 containing a polymerizable compound (L1-5) represented by Formula L1-5 above and a polymerizable compound (L2-5) represented by Formula L2-5 above was prepared by the same method as in Example 1, except that 1,4-naphthalenedicarboxylic acid was used instead of the 4-(4-carboxycyclohexyl)cyclohexanecarboxylic acid. Ratios of the first polymerizable compound (L1-5) and the second polymerizable compound (L2-5) contained in the liquid crystal composition 8 were 87% and 13%, respectively.

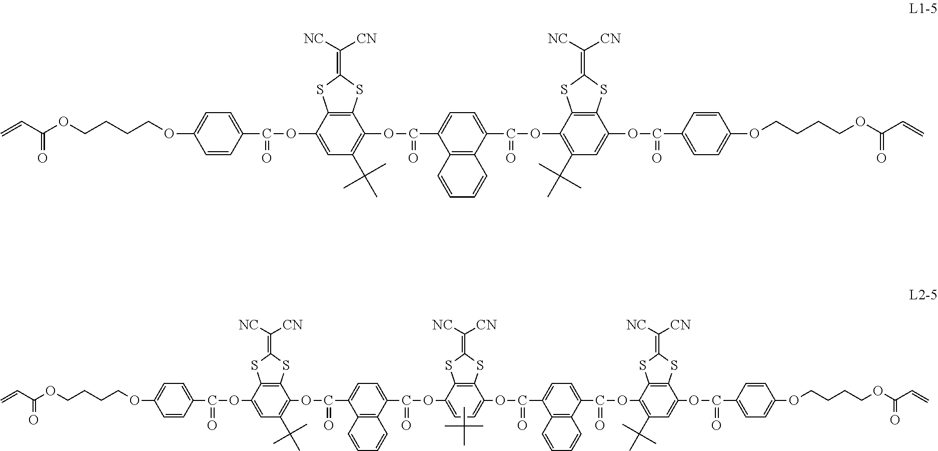

### Example 9

A liquid crystal composition 9 containing a polymerizable compound (L1-6) represented by Formula L1-6 above and a polymerizable compound (L2-6) represented by Formula L2-6 above was prepared by the same method as in Example 1, except that 2-(4,7-dihydroxy-5-methyl-1,3-benzodithiol-2-ylidene)propanedinitrile was used instead of the SM-C. Ratios of the first polymerizable compound (L1-6) and the second polymerizable compound (L2-6) contained in the liquid crystal composition 9 were 95% and 5%, respectively.

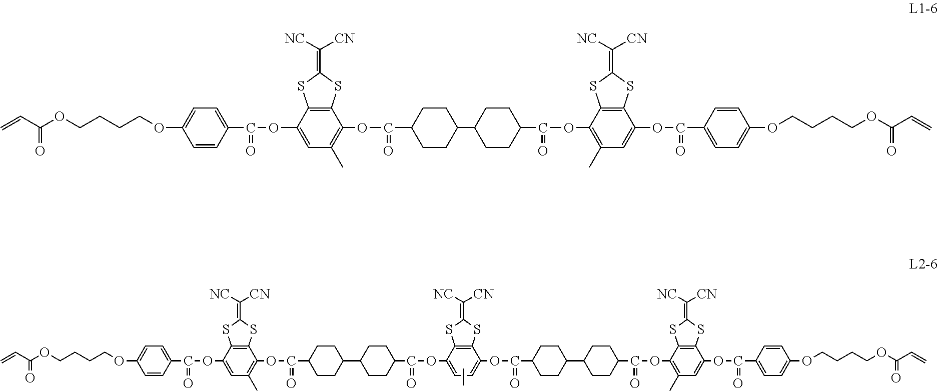

### Example 10

A liquid crystal composition 10 containing a polymerizable compound (L1-7) represented by Formula L1-7 above and a polymerizable compound (L2-7) represented by Formula L2-7 above was prepared by the same method as in Example 1, except that succinic acid was used instead of the 4-(4-carboxycyclohexyl)cyclohexanecarboxylic acid. Ratios of the first polymerizable compound (L1-7) and the second polymerizable compound (L2-7) contained in the liquid crystal composition 10 were 88% and 12%, respectively.

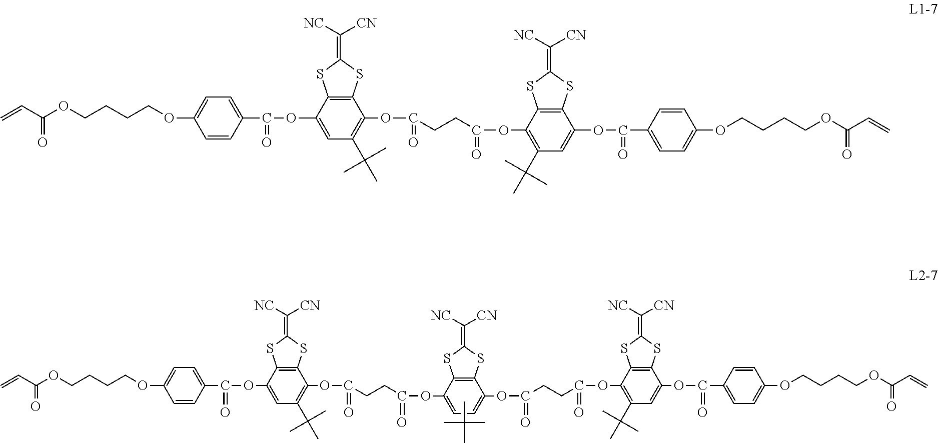

### Example 11

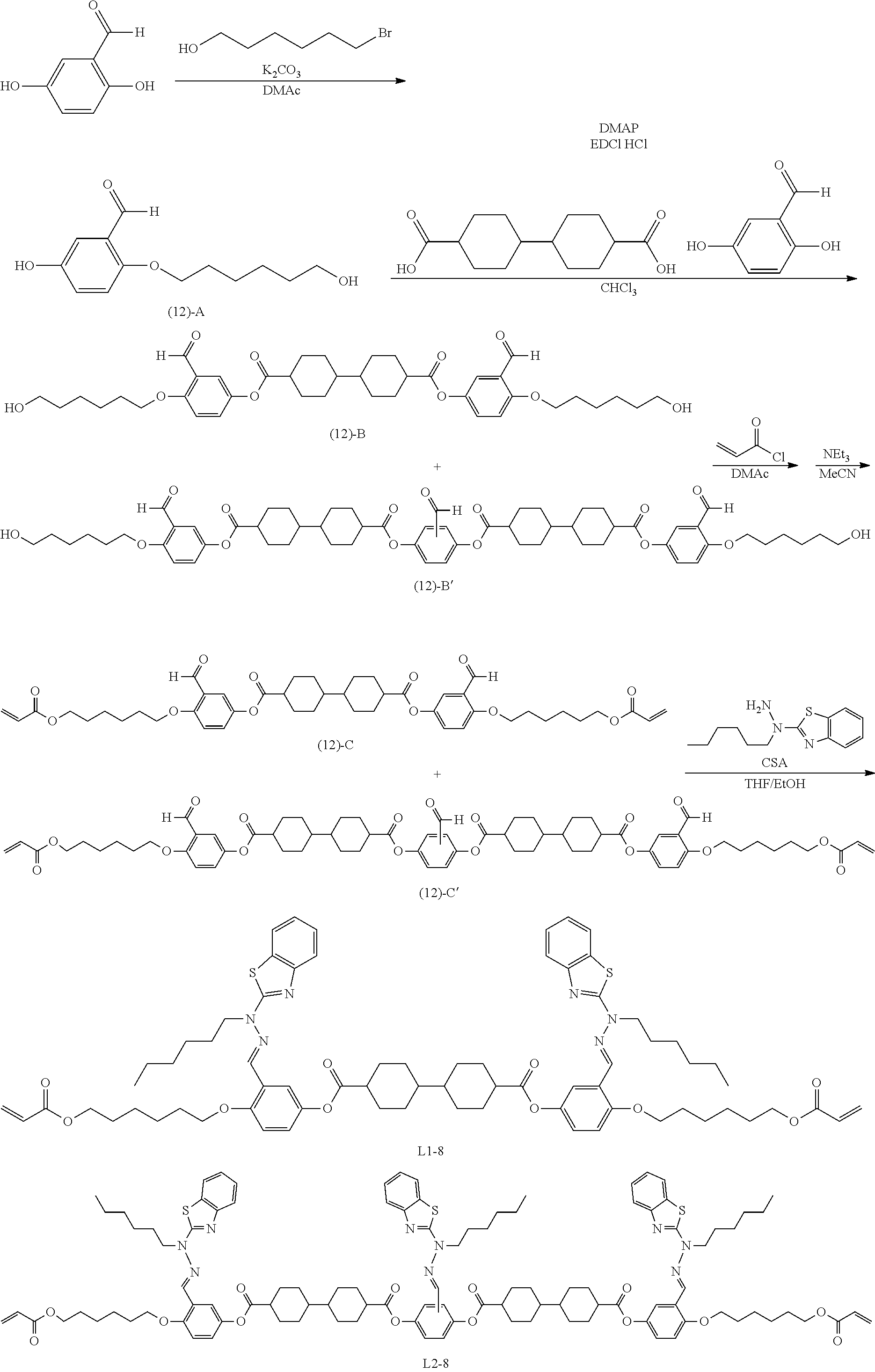

As shown in the above scheme, under a nitrogen atmosphere, 6.08 g of 2,5-hydroxybenzaldehyde and 80 mL of N,N-dimethylacetamide were charged into a 300 mL three-neck flask, 6.0 mL of 6-bromo-1-hexanol was added dropwise thereto while stirring at room temperature, and 6.62 g of potassium carbonate was added thereto. After stirring at 80° C. for 4 hours, the mixture was cooled to room temperature, 100 mL of water and 100 mL of ethyl acetate were added thereto, and the mixture was stirred at room temperature. The organic layer recovered by liquid separation was concentrated, 100 mL of 1 M diluted hydrochloric acid and 100 mL of ethyl acetate were added thereto, and the mixture was stirred at room temperature. The organic layer recovered by liquid separation was concentrated and then purified by silica gel column chromatography (developing solvent: hexane/ethyl acetate) to obtain 3.26 g of a compound (12)-A represented by Formula (12)-A above.

1.21 g of the compound (12)-A and 5 mL of chloroform were charged into a 100 mL three-neck flask, 0.15 equivalent of 2,5-hydroxybenzaldehyde with respect to (12)-A, 0.25 g of (trans, trans)-[1,1′-bicyclohexyl]-4,4′-dicarboxylic acid, 0.03 g of 4-dimethylaminopyridine, 1.17 g of 1-(3-dimethylaminopropyl)-3-ethylcarbodiimide hydrochloride, and 5 mL of chloroform were added thereto, and the mixture was stirred at room temperature for 3 hours. The reaction solution was concentrated and then purified by silica gel column chromatography (developing solvent: hexane/ethyl acetate) to obtain 0.61 g of a mixture of Formulae (12)-B and (12)-B′ above.

0.61 g of the mixture of the compound (12)-B and the compound (12)-B′ and 10 mL of N,N-dimethylacetamide were charged into a 300 mL three-neck flask, and the mixture was stirred under ice cooling in a nitrogen atmosphere. 1.4 mL of acryloyl chloride was added dropwise thereto, the temperature was raised to room temperature, and the mixture was further stirred for 1 hour. 30 mL of water and 30 mL of ethyl acetate were added to the reaction solution, and the organic layer obtained by extraction and liquid separation was concentrated and then purified by silica gel column chromatography (developing solvent: hexane/ethyl acetate) and crystallization (chloroform/methanol) to obtain 0.30 g of a mixture of a compound represented by (12)-C represented by Formula (12)-C and Formula (12)-C′ above.

0.30 g of the mixture of the compound (12)-C and the compound (12)-C′, 0.21 g of 2-(1-hexylhydrazino) benzothiazole, 0.12 g of (+)-10-camphorsulfonic acid, 6 mL of tetrahydrofuran, and 3 mL of ethanol were charged into a 100 mL three-neck flask, and the mixture was stirred at 50° C. for 1 hour. The reaction solution was concentrated and then purified by silica gel column chromatography (developing solvent: hexane/chloroform) and crystallization (chloroform/methanol) to prepare a liquid crystal composition 11 containing a polymerizable compound (L1-8) represented by L1-8 above and a polymerizable compound (L2-8) represented by Formula L2-8 above. Ratios of the first polymerizable compound (L1-8) and the second polymerizable compound (L2-8) contained in the liquid crystal composition 11 were 88% and 12%, respectively.

### Comparative Example 1

A polymerizable compound (I-1-1) represented by Formula I-1-1 was synthesized according to a method described in paragraphs to of JP6823011B.

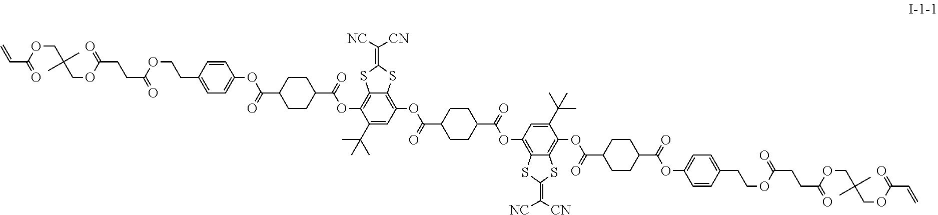 [SMILES: C=CC(=O)OCC(C)(C)COC(=O)CCC(=O)OCCc1ccc(OC(=O)C2CCC(C(=O)Oc3c(C(C)(C)C)cc(OC(=O)C4CCC(C(=O)Oc5cc(C(C)(C)C)c(OC(=O)C6CCC(C(=O)Oc7ccc(CCOC(=O)CCC(=O)OCC(C)(C)COC(=O)C=C)cc7)CC6)c6c5SC(=C(C#N)C#N)S6)CC4)c4c3SC(=C(C#N)C#N)S4)CC2)cc1]

### Comparative Example 2

A liquid crystal composition H2 was prepared by mixing the polymerizable compound (I-1-1) synthesized in Comparative Example 1, a second polymerizable compound (L-1) represented by Formula L-1 below, and a second polymerizable compound (L-2) represented by Formula L-2 below at a ratio of 1/1/1.

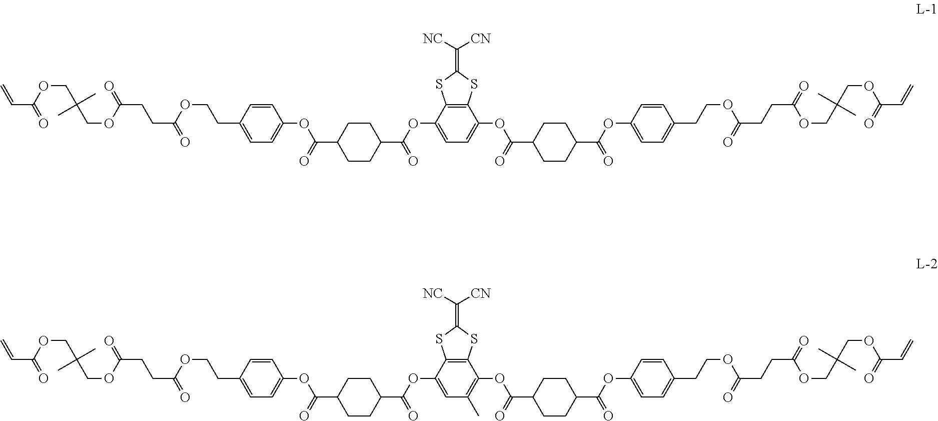

### Evaluation

### (1) Liquid Crystallinity

A phase transition temperature of the prepared liquid crystal composition was measured using a polarization microscope.

Regarding the notation in Table 10 below, Cr indicates a crystal, N indicates a nematic phase, and Iso indicates an isotropic liquid state; and for example, the notation “Iso 226 Ne” indicates that the phase transition temperature between the isotropic liquid and the nematic phase was 226° C.

### (2) Solubility

50 mg of each of the liquid crystal compositions prepared in a 1.5 mL sample bottle was weighed, and a solvent (mixture of cyclopentanone/methyl ethyl ketone at a weight ratio of 3/1) was added thereto until the solid content was 30 wt % (75 mg).

Next, the mixture was heated to 60° C. and stirred to be dissolved, cooled to room temperature (25° C.), and the presence or absence of precipitation was visually confirmed over time after cooling and evaluated according to the following standard. The results are shown in Table 10.

A: no precipitation occurred for 2 weeks (336 hours) or longer at room temperature.

B: no precipitation occurred for 24 hours or longer and shorter than 336 hours at room temperature.

C: precipitation occurred within 24 hours at room temperature.

### (3) Production of Optical Film

A coating liquid for an optically anisotropic film, having the following formulation, was prepared, and a glass substrate provided with a rubbing-treated polyimide alignment film (SE-150 manufactured by Nissan Chemical Corporation) was coated with the coating liquid by spin coating. The coating film was heated and subjected to an alignment treatment on a hot plate to form an optically anisotropic film.

Next, the temperature was lowered, and the alignment was immobilized by irradiation with ultraviolet rays of 1,000 mJ/cm² to form an optically anisotropic film, thereby obtaining an optical film.

In Comparative Example 1, instead of the liquid crystal composition, the polymerizable compound (I-1-1) was used as the coating liquid for forming an optically anisotropic film, but the obtained optical film could not be evaluated as described later.

### Coating Liquid for Optically Anisotropic Film

| | |
|---|---|
| Each of prepared liquid crystal compositions | 15.00 parts by mass |
| Fluorine-containing compound A shown below | 0.12 parts by mass |
| Chloroform | 35.00 parts by mass |

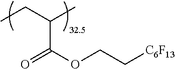

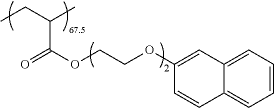

Δn

Using the produced optical film, an in-plane phase difference Re of the retardation layer at 550 nm was measured.

Here, since there is a relationship of a retardation Re(λ)=a birefringence index Δn(λ)×a film thickness d, a birefringence index Δn at 550 nm was calculated from the in-plane retardation Re of the retardation layer at 550 nm and the film thickness d, and evaluated according to the following standard. The results are shown in Table 10.

A: 0.07≤Δn

B: 0.055≤Δn<0.07

C: Δn<0.055

### Reverse Wavelength Dispersibility

Using the produced optical film, an in-plane retardation at a wavelength of 450 nm and a wavelength of 550 nm was measured using Axo Scan (OPMF-1, manufactured by Axometrics, Inc.), and Re(450)/Re(550) was evaluated by the following indicator. The results are shown in Table 10.

A: Re(450)/Re(550)<0.80

B: 0.80≤Re(450)/Re(550)<0.95

C: 0.95≤Re 450)/Re(550)

### Moist Heat Resistance

Each of the produced optical films was bonded to a glass plate with the optically anisotropic film side facing the glass side through a pressure sensitive adhesive.

Next, moist heat resistance of a retardation value was evaluated using Axo Scan (OPMF-1, manufactured by Axometrics, Inc.) according to the following indicator. The results are shown in Table 10.

In a case where a test condition was evaluated as “A” in a test in which the test sample was left to stand in an environment of 100° C. and a relative humidity of 95% for 120 hours, it can be determined that the moist heat resistance was favorable.

A: the amount of change in value after the test with respect to the initial retardation value was less than 10% of the initial value.

B: the amount of change in value after the test with respect to the initial retardation value was 10% or more and less than 20% of the initial value.

C: the amount of change in value after the test with respect to the initial retardation value was 20% or more of the initial value.

### Light Resistance

The produced optical film was subjected to a test in which a glass substrate was set in a xenon irradiation machine (SX75 manufactured by Suga Test Instruments Co., Ltd.) so that the coating film of the liquid crystal composition was an irradiation surface, and irradiated for 200 hours using a #275 filter.

The Re(550) of the optical film before the test and the Re(550) of the optical film after the test were measured, and light resistance was evaluated according to the following standard. The results are shown in Table 10.

A: amount of change of Re(550) after the test with respect to Re(550) before the test was less than 5% of Re(550) before the test.

B: amount of change of Re(550) after the test with respect to Re(550) before the test was 5% or more and less than 15% of Re (550) before the test.

C: amount of change of Re(550) after the test with respect to Re(550) before the test was 15% or more of Re(550) before the test.

### Amine Resistance

With regard to test conditions for amine resistance, a test was performed in which a 2 mol % solution of NH₃/MeOH was put in a vial, the optical film was placed on an outlet portion of the vial, and the vial was allowed to stand for 10 hours.

Re(550) of the optical film before the test and Re(550) of the optical film after the test were measured, and amine resistance was evaluated according to the following standard. The results are shown in Table 10.

A: amount of change of Re(550) after the test with respect to Re(550) before the test was less than 10% of Re(550) before the test.

B: amount of change of Re(550) after the test with respect to Re(550) before the test was 10% or more and less than 30% of Re(550) before the test.

C: amount of change of Re(550) after the test with respect to Re(550) before the test was 30% or more of Re(550) before the test.

### Alignment Temperature

The coating film obtained by spin-coating the coating liquid for an optically anisotropic film used in the production of the optical film was heated on a hot stage heated to a constant temperature (60° C. or higher and in increments of 10° C.) under observation with a polarization microscope, and a time (alignment time) until a uniform alignment state was obtained was measured. A temperature at which the alignment time was 60 seconds or less was defined as an alignment temperature, and the evaluation was performed based on the following standard. The results are shown in Table 10.

A: alignment temperature was lower than 150° C.

B: alignment temperature was 150° C. or higher and lower than 180° C.

C: alignment temperature was 180° C. or higher.

TABLE 10

| | | | Optical performance | | | | | |
|---|---|---|---|---|---|---|---|---|
| | | | | Reverse | Durability | | | |
| | | | | wavelength | Moist heat | Light | Amine | Alignment |
| | Liquid crystallinity | Solubility | Δn | dispersibility | resistance | resistance | resistance | temperature |
| Example 1 | Iso 226 Ne | A | A | A | A | A | A | B |
| Example 2 | Iso 220 Ne | A | A | A | A | A | A | A |
| Example 3 | Iso 195 Ne | A | A | A | A | A | A | A |
| Example 4 | Iso 235 Ne | A | A | A | A | A | A | B |
| Example 5 | Iso 180 Ne | A | A | A | A | A | A | A |
| Example 6 | Iso 225 Ne | A | A | A | A | A | B | A |
| Example 7 | Iso 165 Ne | A | A | A | A | A | B | A |
| Example 8 | Iso 166 Ne | A | A | B | A | A | A | A |
| Example 9 | Iso 245 Ne | B | A | A | A | A | B | B |
| Example 10 | Iso 110 Ne | A | A | A | A | A | A | A |
| Example 11 | Iso 137 Ne 100 Cr | A | A | A | A | B | A | A |
| Comparative | Iso 178 Cr | C | — | — | — | — | — | — |
| Example 1 | | | | | | | | |
| Comparative | Iso 135 Ne 90 Cr | C | B | B | B | A | C | A |
| Example 2 | | | | | | | | |

From the results shown in Table 10, it was found that, in a case where the liquid crystal composition in which the polymerizable compound (I-1-1) represented by Formula I-1-1, disclosed in JP6823011B, was blended and the second polymerizable compound represented by Formula (2) above was not blended, the amine resistance of the formed optically anisotropic film was deteriorated (Comparative Example 2).

On the other hand, it was found that, in a case where the liquid crystal composition in which the first polymerizable compound represented by Formula (1) above and the second polymerizable compound represented by Formula (2) above were blended was used, the amine resistance of the formed optically anisotropic film was improved (Examples 1 to 11).

From the comparison between Examples 1 to 10 and Example 11, it was found that, in a case where Ar¹ and Ar² in Formula (1) represented any aromatic ring selected from the group consisting of the groups represented by Formulae (Ar-1) to (Ar-4) above, the light resistance of the formed optically anisotropic film was improved.

From the comparison between Example 1 and Example 6, it was found that, in a case where Z² in Formulae (Ar-1) to (Ar-8) above represented a tert-butyl group, the amine resistance of the formed optically anisotropic film was improved.

In addition, from the comparison of Examples 1, 2, and 4, it was found that, in a case where the content of the second polymerizable compound represented by Formula (2) above was 15% to 30% by mass with respect to the total mass of the first polymerizable compound represented by Formula (1) above and the second polymerizable compound represented by Formula (2) above, the alignment temperature of the liquid crystal composition was lowered.

### EXPLANATION OF REFERENCES

10: optical film
12: optically anisotropic film
14: alignment film
16: support
18: hardcoat layer

What is claimed is:

1. A liquid crystal composition comprising:
a first polymerizable compound represented by Formula (1); and
a second polymerizable compound represented by Formula (2), wherein a content of the second polymerizable compound is 1% by mass or more with respect to a total mass of the first polymerizable compound and the second polymerizable comnonnd 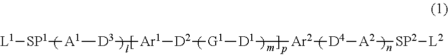
in Formula (1),
D¹, D², D³, and D⁴ each independently represent a single bond, —CO—, —O—, —S—, —C(═S)—, —CR¹R²—, —CR³═CR⁴—, —NR⁵—, or a divalent linking group consisting of a combination of two or more of these groups, where R¹ to R⁵ each independently represent a hydrogen atom, a fluorine atom, or an alkyl group having 1 to 12 carbon atoms,
G¹ represents A^{G} or SP^{G},
A¹, A², and A^{G} each independently represent an aromatic hydrocarbon ring which may have a substituent, an aromatic heterocyclic ring which may have a substituent, or a divalent alicyclic hydrocarbon group which may have a substituent, provided that one or more of —CH₂—'s constituting the alicyclic hydrocarbon group may be substituted with —O—, —S—, or —NH—, SP¹, SP², and SP^{G} each independently represent a single bond or a divalent aliphatic hydrocarbon group having 1 to 20 carbon atoms, provided that one or more of —CH₂—'s constituting the aliphatic hydrocarbon group may be substituted with —O—, —S—, —NH—, —N(Q)—, or —CO—, Q represents a substituent,
L¹ and L² each independently represent a monovalent organic group, where at least one of L¹ or L² represents a polymerizable group, provided that, in a case where at least one of Ar¹ or Ar² is an aromatic ring represented by Formula (Ar-4), at least one of L¹, L², or L³ or L⁴ in Formula (Ar-4) represents a polymerizable group,
m represents an integer of 0 to 2, where, in a case where m is 2, a plurality of G¹'s may be the same or different from each other, and a plurality of D¹'s may be the same or different from each other,
1 and n each independently represent 0 or an integer of 1 or more, where, in a case where 1 is an integer of 2 or more, a plurality of A¹'s may be the same or different from each other, a plurality of D³'s may be the same or different from each other, and in a case where n is an integer of 2 or more, a plurality of D⁴'s may be the same or different from each other, and a plurality of A²'s may be the same or different from each other,
p represents 1,
provided that an aspect in which all of G¹, D¹, and D² represent a single bond and an aspect in which D² represents a single bond and m represents 0 are excluded, and Ar¹ and Ar² each independently represent any aromatic ring selected from the group consisting of groups represented by Formulae (Ar-1) to (Ar-8), 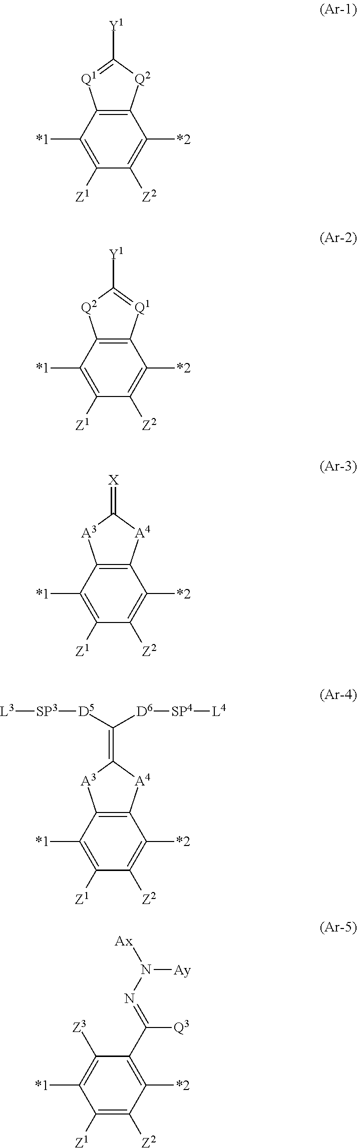 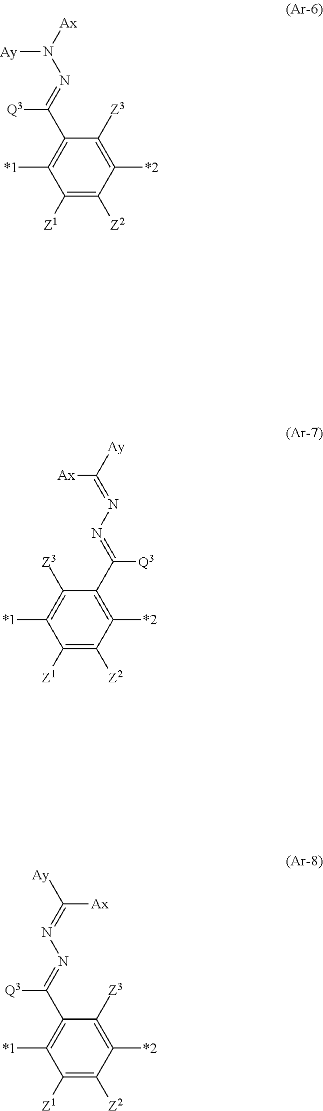
in Formulae (Ar-1) to (Ar-8),
*1 represents a bonding position to D³ or D⁴, and *2 represents a bonding position to D¹ or D², provided that, in a case where 1 is 0, the bonding position to D³ represents a bonding position to SP1, in a case where m is 0, the bonding position to D¹ represents a bonding position to D², and in a case where n is 0, the bonding position to D⁴ represents a bonding position to SP²,
Q¹ represents N or CH,
Q² represents —S—, —O—, or —N(R⁶)—, where R⁶ represents a hydrogen atom or an alkyl group having 1 to 6 carbon atoms,
Y¹ represents an aromatic hydrocarbon group having 6 to 12 carbon atoms, which may have a substituent, an aromatic heterocyclic group having 3 to 12 carbon atoms, which may have a substituent, or an alicyclic hydrocarbon group having 6 to 20 carbon atoms, which may have a substituent, provided that one or more of —CH₂—'s constituting the alicyclic hydrocarbon group may be substituted with —O—, —S—, or —NH—,
Z¹, Z², and Z³ each independently represent a hydrogen atom, a monovalent aliphatic hydrocarbon group having 1 to 20 carbon atoms, a monovalent alicyclic hydrocarbon group having 3 to 20 carbon atoms, a monovalent aromatic hydrocarbon group having 6 to 20 carbon atoms, a monovalent aromatic heterocyclic group having 6 to 20 carbon atoms, a halogen atom, a cyano group, a nitro group,-OR⁷,-NR⁸R⁹,-SR¹⁰,-COOR¹¹, or-COR¹², where R⁷ to R¹² each independently represent a hydrogen atom or an alkyl group having 1 to 6 carbon atoms, and Z¹ and Z² may be bonded to each other to form an aromatic ring,
A³ and A⁴ each independently represent a group selected from the group consisting of —O—, —N(R¹³)—, —S—, and —CO—, where R¹³ represents a hydrogen atom or a substituent,
X represents a non-metal atom of Groups 14 to 16, provided that a hydrogen atom or a substituent may be bonded to the non-metal atom,
D⁵ and D⁶ each independently represent a single bond, —CO—, —O—, —S—, —C(═S)—, —CR¹R²—, —CR³═CR⁴—, —NR⁵—, or a divalent linking group consisting of a combination of two or more of these groups, where R¹ to R⁵ each independently represent a hydrogen atom, a fluorine atom, or an alkyl group having 1 to 12 carbon atoms,
SP³ and SP⁴ each independently represent a single bond or a divalent aliphatic hydrocarbon group having 1 to 20 carbon atoms, provided that one or more of —CH₂—'s constituting the aliphatic hydrocarbon group may be substituted with —O—, —S—, —NH—, —N(Q)—, or —CO—, Q represents a substituent,
L³ and L⁴ each independently represent a monovalent organic group, where at least one of L³, L⁴, or L¹ or L² in Formula (1) represents a polymerizable group,
Ax represents an organic group having 2 to 30 carbon atoms, which has at least one aromatic ring selected from the group consisting of an aromatic hydrocarbon ring and an aromatic heterocyclic ring,
Ay represents a hydrogen atom, an alkyl group having 1 to 12 carbon atoms, which may have a substituent, or an organic group having 2 to 30 carbon atoms, which has at least one aromatic ring selected from the group consisting of an aromatic hydrocarbon ring and an aromatic heterocyclic ring,
where the aromatic rings in Ax and Ay may have a substituent, and Ax and Ay may be bonded to each other to form a ring, and
Q³ represents a hydrogen atom or an alkyl group having 1 to 20 carbon atoms, which may have a substituent, 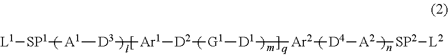
in Formula (2), D¹, D², D³, D⁴, G¹, A¹, A², SP¹, SP², L¹, L², m, l, n, Ar¹, and Ar² each have the same meaning as described in Formula (1), and
q represents an integer of 2 to 9, where a plurality of Ar¹'s in Formula (2) may be the same or different from each other, a plurality of D²'s may be the same or different from each other, a plurality of G¹'s may be the same or different from each other, and a plurality of D¹'s may be the same or different from each other.

2. The liquid crystal composition according to claim 1,
wherein Ar¹ and Ar² in Formulae (1) and (2) represent any aromatic ring selected from the group consisting of the groups represented by Formulae (Ar-1) to (Ar-4).

3. The liquid crystal composition according to claim 1,
wherein G¹ in Formulae (1) and (2) represents A^{G}.

4. The liquid crystal composition according to claim 1,
wherein any one of Z¹ or Z² in Formulae (Ar-1) to (Ar-8) represents a tert-butyl group.

5. The liquid crystal composition according to claim 1,
wherein G¹ in Formulae (1) and (2) represents a cycloalkane ring or a cycloalkene ring.

6. The liquid crystal composition according to claim 1,
wherein, in Formulae (Ar-1) to (Ar-8), Z¹ represents a hydrogen atom and Z² represents a tert-butyl group.

7. The liquid crystal composition according to claim 1,
wherein, in Formulae (1) and (2), both of 1 and n represent 1, and both of A¹ and A² represent a benzene ring.

8. The liquid crystal composition according to claim 1,
wherein the content of the second polymerizable compound is 5% to 50% by mass with respect to the total mass of the first polymerizable compound and the second polymerizable compound.

9. The liquid crystal composition according to claim 1, further comprising:
a polymerizable compound having one or more polymerizable groups, which is different from the first polymerizable compound and the second polymerizable compound.

10. An optically anisotropic film obtained by fixing an alignment state of the liquid crystal composition according to claim 1.

11. An optical film comprising:
the optically anisotropic film according to claim 10.

12. A polarizing plate comprising:
the optical film according to claim 11; and
a polarizer.

13. An image display apparatus comprising:
the optical film according to claim 11.

14. An image display apparatus comprising:
the polarizing plate according to claim 12.

15. A polymerizable compound represented by Formula (2), 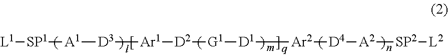
in Formula (2),
D¹, D², D³, and D⁴ each independently represent a single bond, —CO—, —O—, —S—, —C(═S)—, —CR¹R²—, —CR³═CR⁴—, —NR⁵—, or a divalent linking group consisting of a combination of two or more of these groups, where R¹ to R⁵ each independently represent a hydrogen atom, a fluorine atom, or an alkyl group having 1 to 12 carbon atoms,
G¹ represents A^{G} or SP^{G},
A¹, A², and A^{G} each independently represent an aromatic hydrocarbon ring which may have a substituent, an aromatic heterocyclic ring which may have a substituent, or a divalent alicyclic hydrocarbon group which may have a substituent, provided that one or more of —CH₂—'s constituting the alicyclic hydrocarbon group may be substituted with —O—, —S—, or —NH—,
SP¹, SP², and SP^{G} each independently represent a single bond or a divalent aliphatic hydrocarbon group having 1 to 20 carbon atoms, provided that one or more of —CH₂—'s constituting the aliphatic hydrocarbon group may be substituted with —O—, —S—, —NH—, —N(Q)—, or —CO—, Q represents a substituent,
L¹ and L² each independently represent a monovalent organic group, where at least one of L¹ or L² represents a polymerizable group, provided that, in a case where at least one of Ar¹or Ar² is an aromatic ring represented by Formula (Ar-4), at least one of L¹, L², or L³ or L⁴ in Formula (Ar-4) represents a polymerizable group,
m represents an integer of 0 to 2, where, in a case where m is 2, a plurality of G¹'s may be the same or different from each other, and a plurality of D¹'s may be the same or different from each other,
1 and n each independently represent 0 or an integer of 1 or more, where, in a case where 1 is an integer of 2 or more, a plurality of A¹'s may be the same or different from each other, a plurality of D³'s may be the same or different from each other, and in a case where n is an integer of 2 or more, a plurality of D⁴'s may be the same or different from each other, and a plurality of A²'s may be the same or different from each other,
q represents an integer of 2 to 9, where a plurality of Ar¹'s may be the same or different from each other, a plurality of D²'s may be the same or different from each other, a plurality of G¹'s may be the same or different from each other, and a plurality of D¹'s may be the same or different from each other,
provided that an aspect in which all of G¹, D¹, and D² represent a single bond and an aspect in which D² represents a single bond and m represents 0 are excluded, and Ar¹ and Ar² each independently represent any aromatic ring selected from the group consisting of groups represented by Formulae (Ar-1) to (Ar-8), 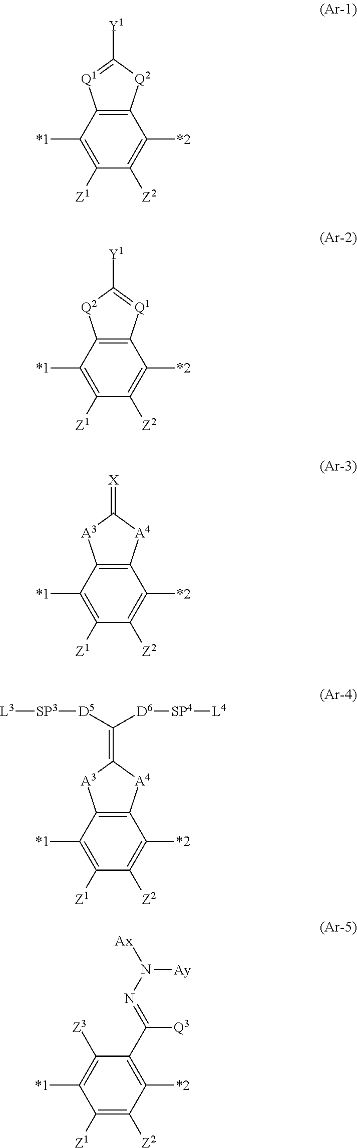 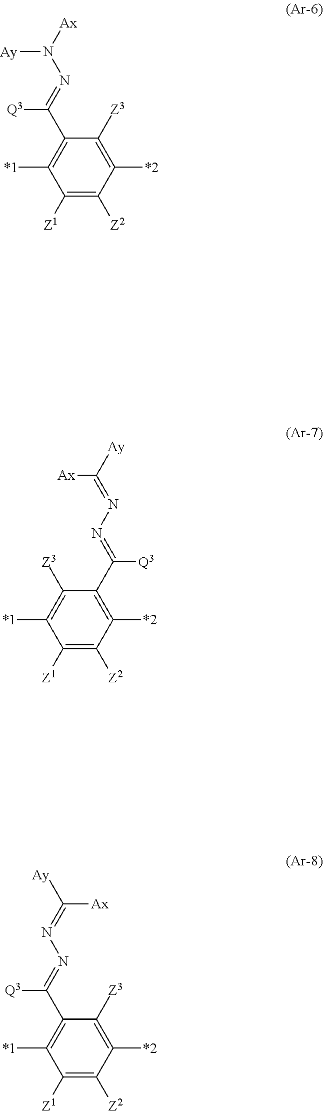
in Formulae (Ar-1) to (Ar-8),
*1 represents a bonding position to D³ or D⁴, and *2 represents a bonding position to D¹ or D², provided that, in a case where 1 is 0, the bonding position to D³ represents a bonding position to SP¹, in a case where m is 0, the bonding position to D¹ represents a bonding position to D², and in a case where n is 0, the bonding position to D⁴ represents a bonding position to SP²,
Q¹ represents N or CH,
Q² represents —S—, —O—, or —N(R⁶)—, where R⁶ represents a hydrogen atom or an alkyl group having 1 to 6 carbon atoms,
Y¹ represents an aromatic hydrocarbon group having 6 to 12 carbon atoms, which may have a substituent, an aromatic heterocyclic group having 3 to 12 carbon atoms, which may have a substituent, or an alicyclic hydrocarbon group having 6 to 20 carbon atoms, which may have a substituent, provided that one or more of —CH₂—'s constituting the alicyclic hydrocarbon group may be substituted with —O—, —S—, or —NH—,
Z¹, Z², and Z³ each independently represent a hydrogen atom, a monovalent aliphatic hydrocarbon group having 1 to 20 carbon atoms, a monovalent alicyclic hydrocarbon group having 3 to 20 carbon atoms, a monovalent aromatic hydrocarbon group having 6 to 20 carbon atoms, a monovalent aromatic heterocyclic group having 6 to 20 carbon atoms, a halogen atom, a cyano group, a nitro group, —OR⁷, —NR⁸R⁹, —SR¹⁰, —COOR¹¹, or —COR¹², where R⁷ to R¹² each independently represent a hydrogen atom or an alkyl group having 1 to 6 carbon atoms, and Z¹ and Z² may be bonded to each other to form an aromatic ring,
A³ and A⁴ each independently represent a group selected from the group consisting of —O—, —N(R¹³)—, —S—, and —CO—, where R¹³ represents a hydrogen atom or a substituent,
X represents a non-metal atom of Groups 14 to 16, provided that a hydrogen atom or a substituent may be bonded to the non-metal atom,
D⁵ and D⁶ each independently represent a single bond, —CO—, —O—, —S—, —C(═S)—, —CR¹R²—, —CR³=CR⁴—, —NR⁵—, or a divalent linking group consisting of a combination of two or more of these groups, where R¹ to R⁵ each independently represent a hydrogen atom, a fluorine atom, or an alkyl group having 1 to 12 carbon atoms, SP³ and SP⁴ each independently represent a single bond or a divalent aliphatic hydrocarbon group having 1 to 20 carbon atoms, provided that one or more of —CH₂—'s constituting the aliphatic hydrocarbon group may be substituted with —O—, —S—, —NH—, —N(Q)—, or —CO—, Q represents a substituent,
L³ and L⁴ each independently represent a monovalent organic group, where at least one of L³, L⁴, or L¹ or L² in Formula (1) represents a polymerizable group,
Ax represents an organic group having 2 to 30 carbon atoms, which has at least one aromatic ring selected from the group consisting of an aromatic hydrocarbon ring and an aromatic heterocyclic ring,
Ay represents a hydrogen atom, an alkyl group having 1 to 12 carbon atoms, which may have a substituent, or an organic group having 2 to 30 carbon atoms, which has at least one aromatic ring selected from the group consisting of an aromatic hydrocarbon ring and an aromatic heterocyclic ring,
where the aromatic rings in Ax and Ay may have a substituent, and Ax and Ay may be bonded to each other to form a ring, and
Q³ represents a hydrogen atom or an alkyl group having 1 to 20 carbon atoms, which may have a substituent.
